SEQUENCE LISTING (141 sequences)

SEQ ID NO: 1 - protein, 438 aa, Chlamydomonas reinhardtii
MQMQANRSSLRASPVRGLGARPLLRALPAGRVARLNVSAQAKDPNAPIQSNPLGTLSSQSGQVATLPRSEEARKYFRTVYDFPQWQKHRSSYRFAERLFQLSQSHILQNALPAISWVTLVATLVASYGYSYDQHMLPDVFPSISPNASCTAFISNTSVALSLLLVFRTNSSYGRWDEARKMWGGLLNRSRDIMRQGATCFPDDQVEAKKALARWTVAFSRALRIHFQPEVTIESELQNILTPAELQMLAKSQHRPVRAIHAISQIIQSVPMSSIHQQQMSNNLTFFHDVLGGCERLLRAPIPVSYTRHTARFLFAWLTLLPFALYPTTGWGVVPVCTGIAAVLCGIEEIGVQCEEPFGILPLDVICNRIQADVMATLKDDADTKTILAEAGLISLIPSATSATPVASAEPVLVSARPSAAPAPNNGLQVRVAMGGERK

SEQ ID NO: 2 - protein, 450 aa, Chlamydomonas reinhardtii
MQCLSSRPVAMGRAGSSALPRLPLRAGRVCHLGVRCQAANKDPNAPIQSNPLGSFSSQLQNQPTLPRSEEARKYFRTVYDFPQWQTHRNQYRLMKRLFSIPQSHVIQNALPSIMWVAFTSTCVAAYMYGYDQHMLPEGFPTLAPNAACSAFISNTSVALSLLLVFRTNSSYGRWDEARKMWGGLLNRSRDIMRQGATCFPDDQVEAKKALARWVVAFSRALRIHFQPEVTIESELKNILTPAELQMLAKSQHRPVRAIHAISQIIQSVPMSSIHQQQMSNNLTFFHDVLGGCERLLRAPIPVSYTRHTARFLFAWLTLLPFALYGSCGVSVIPVCSGIAAVLCGIEEIGVQCEEPFGILPLDVICNRIQADVMATLKDDADTKTILAEAGLISLRANSAMAVENALPDLDSINAAAPNGNGSHNGNGAAVPVSVSAGASGNGMNVRISPR

SEQ ID NO: 3 - protein, 466 aa, Chlamydomonas reinhardtii
MQVSKVPSSASARCLPRLPVRTSRVCQLSVRCQAANKDPNAPIQSNPLGSFSSQNSSGAVVTAPRNEDARKYFRTVYDFPQWQKHRSQSRLVRRLFTIPQSHVIQNALPSIMWVTFTSTCVAAYMYGYDLHILPEGFPTLAPNAACSAFISNTSVALSLLLVFRTNSSYGRWDEARKMWGGLLNRSRDIMRQGATCFPDDQVEAKKALARWTVAFARALRIHFQPEVTIESELQNILTPAELQMLAKSQHRPVRAIHAISQIIQSVRMSSIHQQQMSNNLTFFHDVLGGCERLLRAPIPVSYTRHTARFLFAWLTLLPFALYGSCGVSVIPVCTGIAAVLCGIEEIGVQCEEPFGILPLDVICNRIQADVMATLKDDADTKTVLAEAGLISLIPSMSLPPTEHASPSDPVTAAAAAALAAANGNGAASHSNGNGSKPVSTQVPPPVLAPVTVTSSSGSMNVRISPR

SEQ ID NO: 4 - protein, 325 aa, Helicosporidium sp.
  source 1..325: note = ATCC 50920
MLAATNRSASSPPKPPFEQKGYFPGSWPHATFDLSQGFYSCTFALSLLLVFRTNAAYDRWWEARKTWGSLVNRTRDFMRQALTFFPASESAEAGVLLRWTVALPRMLLWKLREQAVLEAELQGVLLPAEFSLLRECDHRCALALALMSEVVQRARGLDRGQRRCLDANLTALGDTVGVCERILRTPIPLSYSRHTSRWLLVWLAFLPFSLWRLYAWGSVPLSGLLAFLMLGIDEIGVQIEQPFQVLPLESLCCTVERNLRNLAAATPAVKRLASLAVEAQLALAESDATASFREPGKSLACSVFPPVSPRSSLIRSCLDRPQSLP

SEQ ID NO: 5 - protein, 422 aa, Corchorus capsularis
MAQSSNPSKLSLSSDFNPKLFPNKLHRYSSFPFKLSQPRSFKTLSSLSPPPPPSSPPPPSSEPTTTKTLNLVSLLRAIPDWADRVQERGMRQNRALYNHDKWSEHRSSLRHLRHLLSSLQSRVILSLVPPVLAFTSVAAVIATYNTAVDLHWLPGFFPVLRASSLPYQLTAPALALLLVFRTEASYSRFEEGRKAWTKVISGTNDFARQVISGVENSSDDQSLKDALLRYIMAFPVALKCHVMYGSDIGRDLQNLLEVDDLTVVLNSKHRPRCIIDFISHSLRVLNLKESERTVLESKISCFHEGIGVCEQLMGIPIPLSYTRLTSRFLVLWHLTLPIILWDDCHWIVVPATFISAASLFCIEEVGVLIEEPFPMLALDELCNVVQNNIREAIATEKVIKAKLIGKRKRHSYKHSPNGWPNT

SEQ ID NO: 6 - protein, 406 aa, Marchantia polymorpha
  source 1..406: note = subsp. ruderalis
MEDVRLGRCEGAEGVGKMGKWVHKLARFYAPCALPLLCVTMGLWIPESPGSRSGRSGPEPEPYDPDFPKEEVFLYRRTVYDHKDWSRHRSSLRHSRHILSMRSSRVILALWPPVFGLTTVSIALSAYNECILSHWLPSFLPLLHVSATPFQLMAPALALLLVFRTNASYARFDEARRAWGSNVNRTRDITRQALTWMQHPSDAEKVKKLIRHCVAFNVCMKHHLVRGGDLRDDLHSWIDKEEIDGILASTHRPNYVLQVMSEIIHSCSITEMQLTRMDVNMTQFADNLGACERIFKTPIPLSYTRLTSRFLVLWHIALPLALWDHCQWVSVPATFLSAGALFCIEEVGVLIEEPFQIFPLDNICSTIKKNVDGLILAHTEIHHCHKHPPKGCDIPKKKSSGDAPKS

SEQ ID NO: 7 - protein, 453 aa, Micractinium conductrix
MTCLTSVLPLGGALVPTRPLRSGIHSSTPRRRLSALAPLKPHLVAEPESVQHAAPAAAAPPPPPPRDATPSAPRQPLFGDALLESTRPQLRTVFDADRWVMHRSVNRYFRHLWDLPRSRLLLGLAAPLAYVLGLSAVVCAYGAAQQAGLLPSLLPSLAGAARMRELYNLTSPTLALLLVFRTNASYARWDEGRKMWGMVLNRTRNICRLGLAWIGDDKRELRSMLEELEGILLPHEIEGVLRASHRPNYVLQVLAQIVRTAGLPTAATLRMEDDLTNFGDSLGGCERLLRTPIPLFYTRHTSRFLMIWLTFLPATLWPACGLLTLPLVFLISFLLLGVDKIGVSIEEPFSILELETIASRALENVHELAAMHDGVADIPLGGGSSSSSSSNRASSNRGAGTGRLHPSFGVYSSSNGSSGANGSGGHHGSGLIGHVSSIVSAATMVSLARPATA

SEQ ID NO: 8 - protein, 803 aa, Chlorella sorokiniana
MPAALVAAAAATACPAAMGRAPAAAGSASRLPAGLVPSSQQLRWTLAPQRHSQPSRCRRAARVAAVAGPQAPPRLGAPLQLGSPASQKRREEILNRIDTLIQEELESQLPLPSVDELVADDFKESERRKLRTVFDFDLWKRHRSSSRYSRHMFGLLESRTVRWIGAPLAYCVSLSVAVGVYHNLADAGIVPEVLPELKTQLPIQLTSAALSTLLVLRTNTSYQRWDEARKMWGLIVNRSRDFTRQALGYIPDDQPELQDMLCRWVVAYSRSLMCHLRSDEDLYRELATKLKPQELEALMSSSHRPNYTVQVLTAIMQAAKLPGVDIDPRDSGANVKASALVRMDEQLTQFADVTGGCERILRTPVPLAYSRHNSRMLTLWMTLLPFSLWDTCGWATPFVVLIVAFLLLGVKEIGLNIEEPFSILPLETICNTIETNVWELHRTHSTAAAEAQQEAARRGSWSQQAQQGPQQVDADLLAPAAATQLGVALWRFPTGAAYLLSRTNLASLIERNLRLRRQLGISDAATATALFQSQGALVSKFERAEVMVAHLQRLQASGELSAEQVARMALAPSALHLTPAEFDRRWHDGGLSRPGASPTDITMSDTAGRQQAAAAALGELLQAADGVPGPLQPQYLQQGEELVQRSAGLWRAGPAALRAGWASLQQLGLSDSQVVAAMTMQPAVLSLDWAGETKQRLLAWAEEHLGIDTFGFLTSYTRFVTYSGASVAMRADFLQQHHPAMWDQNLCRGPRTLFRLLTEPERLCAHAGCTETELVAFNRAWLATPAGLRWGGKAGRSQRRAAS

SEQ ID NO: 9 - protein, 417 aa, Handroanthus impetiginosus
MKISPSSCSPQFLPRTLFKFHHSPLPFISKKPHKLSFKFICACQSSNDSNPSPSQDFTLLSLLLAIPNWADGIKERRMKQKRSLYDHKSWVQHRSSLRHVRHFLSSLSSRVILSLVPPVIVFTLVAVIIASYNSAVSIHWLPEFFPILRASSLPYQLTAPALALLLVFRTEASYSRFEEGKKAWTKVISGTNDFARQVIASVESPSDSMLKKAILQYIMAFPVALKCHIIYGSDIAQDLKNLLEADDLSVVLSSKHRPRCIIEFISQSLQLLDMDATKLHVLESKLCCFHEGIGVCEQLMGTPIPLSYTRLTSRFLILWHLTLPVILWDDCHWIVVPATFISAASLFCIEEVGVLIEEPFPMLALDELCLTAQSNIQDIMRNEKLIKDQVKKAKRKIHSRKHSSNGWPNPLEDRQPS

SEQ ID NO: 10 - protein, 385 aa, Chlamydomonas eustigma
MLVHRVHTRSLGNRNQCGRKLHRVSTFVVKTPTEKPVVADYVLPRSEEARRYFRTVYDFPQWQKHRSPTRLIDRLLQIPRSHVLQNILPSIAWCSSVAGLLTLYMQAYDAHILPDGFPSFATNNACTSFVNTTTVALSLLLVFRTNVSYGRWDEARKMKGLLVNRSRDLMRQVCAMVPEEDVATKAMMAKWTAAFCRVLRIHFQPEVSLEDEMKGLLSPEELEWLIESKHRPCSVIHMLSQIIYDSQISAICQAQMCNNLTAFEDVLGGCERLLRAPIPVSYTRHTARFLFTWLTLLPFALYNSCGVWTLPVVAGVSAVLCGIEEIGVQIEEPFGILPLEAICGRIQADVMATLKEDAKTRNLRNKVLLIRGPEHWAMVNGHSNK

SEQ ID NO: 11 - protein, 397 aa, Chlamydomonas eustigma
MNKSLLRAPESNVVFRRACNKSFVKRCKEVQRLPSHVALAVKNPAEKPVVADYVLPRSEEARRYFRTVYDFPQWQKHRSQLRLIDRLLQIPRSHVLQNILPSIAWCSSVAGLLTLYMQAYDAHILPDGFPSFTPNNACTSFVNTTTVALSLLLVFRTNVSYGRWDEARKMKGLLVNRSRDLMRQACAMLPAEDIETKAMMAKWTAAFCRVLRIHFQPEVSLEDEMKGLLSPEELQWLQESKHRPCSVIHVLSQIIYDSQISAICQAQMCNNLTAFEDVLGGCERLLRAPIPVSYTRHTARFLFTWLTLLPFALYNSCGVWTLPVVAGVSAVLCGIEEIGVQIEEPFGILPLEAICGRIQADVMATLREDEKMRNLRKRVIQTRTTELRELASGGASK

SEQ ID NO: 12 - protein, 402 aa, Chlamydomonas eustigma
MKVGIHVVQTLSGHTISGQRFLNKAGCNKLRPANKSSFRAQSTSALDEFLHRIEINIDKDKEAARSERRQAFTHVDWKRHRSSARFIRHLSTIPESWVIRGVLWHVACFILLALGVAAFNYSYACKGLVPPYSIAIEPMQLTSFALSLLLVFRTNASYARWQEARRSFGSITTTGKDIMRQSLAWFCEDDWAGKMALSGWLQALAQSSMYHLQNEGHLEDDSLVPLQGCLSPDELAQLRISSHKPTFCIAMITRLVSRAGMPTELLLRMDENISTLVQAVSSCERIINTPIPLSYTRHTSRFLMVWLTCLPFSLWQYCGWAMVPIAGLISFVLLGIEEIGVYIEEPFSLLPLESLCDKLTHSIDNMMLEHRLFSEGEVITPKGSTGDSECLATSSKMQRYNC

SEQ ID NO: 13 - protein, 838 aa, Chlamydomonas eustigma
MFFRKACSQPIGRKHQGGHRFQRLVRPNFNCSVTDADKAAENFVLSRSEESRRYFRTVYDFPLWQKHSNQPRIIDEVRKNSRTNILKSILPSTAWCTGVAALLTAYVQAYDSNMLPAQFPRFESIPSYAAIVNITVVSLSLLLVFRTNASYGQWDAARKMKGLLVNRSQELMREACSLLPAEDTATKIMMGRWISAYAGVLRNQFQPEESLGKIVDGLLSTEELEWLNSSNQRPSSVIDVLSQIISSAAIPAPNQAQMNNNLAALRGVLGGCQRLLKDPIPMSYTRNSARLLFLWLTLLPFALFSSCGVWTAPVVAAVSAVLCSIEEIGVQIEEPSTTGMLPLEAIGDRIQADVITTLLEDEKTKTMRGEVDTAALRAAAQPSKLDEVPGKLVLLAEQVAVVGQSSNNQGTGGAGQKGERRVLLSKLETETKDMKDNLQRLKQRATELSTKAQQASDVARKERANAVLKLGEMDEQLKTSQEAGVYSEITQLKDSLTTRESDVASLVSELMLEQQSNQQLLEQVALLQMQLSETENMLTSEKSIGEGLSQQLQITQVDLNTSQSSLADMSQKLREEEFQRATLQSEIQALRSDLDRVTAVVETRQKEADVYRAAAEKAEQQVVDAAALRQAMMAELTETSDSFAELLAEALSRAESAEADKVKVEAEVRMLQSKIVSADAALSRLETQLLEEERKGIPPSGSNDMEFEDVQKRIAELEGLLGGLLSEVEQATVKQGRTRLTQDTIEEVKREAELRSARVAAELAAESGARSEAEDNLRVLQSELLQLKEASQTSRHAAEKELKEMQLLISEVKAKAAETAAFVGKAAKEGPKKGKSAY

SEQ ID NO: 14 - protein, 365 aa, Chlamydomonas eustigma
MKVCIHVVQTLSGHTISGQRFLNKAGCNKLRPANKSSFRAQSTSALDEFLHRIEINIDKDKEAARSERRQAFTHVDWKRHRSSARFIRHLSTIPESWVIRGVLWHVACFILLALGVAAFNYSYACKGLVPPYSIAIEPMQLTSFALSLLLVFRTNASYARWQEARRSFGSITTTGKDIMRQSLAWFCEDDWAGKMALSGWLQALAQSSMYHLQNEGHLEDDSLVPLQGCLNPDELAQLRISSHKPTFCIAMITRLVSRAGMPTELLLRMDENISTLVQAVSSCERIINTPIPLSYTRHTSRFLMVWLTCLPFSLWQYCGWAMVPIAGLISFVLLGIEEIGVYIEVIIFFLLYLGQLSHNCSHIVK

SEQ ID NO: 15 - protein, 831 aa, Chlamydomonas eustigma
MLVQKTCSKPLSCKQQAGLRTLRIFRYVFQNARQDVKIAVAKVVLPPSEEARRSFRTVYDFQSWRKHRNQLRLFVQVGQIPRSNILKNILPSMAWCTGVAALLTLYMQAYDSNLLPEPFPSFASNTASVTFVNTTVVALSLLLVFRTNASYGRWDEARKMKGLLVNRSRDLIRQACSMFSPEDVERKIMMGKWVSVYCRVLRIHFQAEVSLEEEMNGILSMEEVEWLQSSTHRPCTVIHVLSQIIYSAPISAFCQAQMCNNLTALEDVLGGCERILRAPIPVSYTRNTARFLFVWLTLLPFALFSSCGVWTAPVVAGIAAVLCGIEEIGVQVEEPFGILPLAAIGDRIQADVISTLLEDGRTRTMRSEVNTAALRAMAQPPNQEGVLGSVDRQSTNLANSQESTTGSNPMGQRVVLLSKLETETKDMKDNLNKLKQRATELSMKAQLASEAAQRERASAVQKLGIMNEQLKGFKGAEVYSEIAQLKDTLTSRESDVATVVSEMQQQQLSSQQLFEQLAHLQTQLSEAEALLSTEKSMGQELSQQLQLTQMGLKTNQASLLDVSHKLQKEELQRSAFQTEIQALRRDLDRITTAMESKRLETEAFWAAAESAEQHVADSAALQSAMMAELTETSDAFAERLAAALSRAETAETDKFRAEQEVKMLQSKIADTETMLRKLEAKAAELERKGSAPSESTALEFRDAQWKIAELEGLLGEVLLEVKHAASNNATEYSTQDKIHEVQREADRRSIRVAAEIAAERGARSEAEDNLRILQRELLLLKEDSLSSRNAAERELKEMQLLICEVKAKAASTATFVGTAAAEGPRRGQNTY

SEQ ID NO: 16 - protein, 451 aa, Chlorella variabilis
MTASDAPSASRSGGHARLVQTAASPRGDGSFAVGSTDLEQVAEQAVVDAVAQTAGDRATSSSAAAGSREVDPIKEGSRKYRRTVYDFENWRQHRSTKRYMRHAKGLLGSRIFRGLASPLLYILAVSASVAVWNTLVETGLAPDVLPELHMSNNGPFGLTSFALSLLLVTNASYARWLDARKAWGMLVNRSRDITRQALTCFPAADRPLLDMLCRWTAAYSRALMCHVREDSDLEAELRKVLPAHEVEAVVLAKHRPNYCLQVMSEIVHSAHLSALPLPVARGAVVASGAPALAAAGWEGTRYRMDENLTAMEDILGACERILRAPIPLSYTRHTSRFMMIWLTLLPFSLWDNCGWASVPLCGIIAFLLLGIEEIGVSIEEPFSILPLEVICDIIEANVRELRSIHAGSHAEDGSQPRRAPAARDLVGRAVAEAEQEEAAAAAGTVAAAAGA

SEQ ID NO: 17 - protein, 456 aa, Chlorella variabilis
MASRLHGSRHQGALLGLQQPAGLVPRTRSLNRGRLTQLRVAAAASSPPLPAKLSGDDLKEAELKKLRTVADFEFWKRHRSSSRYWRHVLGIFDSRTFSWVAAPLSYVMLLTTGVCLYYTLAEAGIVPEVIPEISASAAAPFGLTSFALSTLLVLRTNTSYQRWDEARKMWGLIVNRTRDISRQAVGYIPPHQAELQDMFCRWLVAYCRSLMCHLRAGEDLEAELKGKLTDIELKALLASTHRPNYTCQVLTAIIRAAQLPGGKVDMNDSFANVKASAAFRMDENLTQYADVTGGCERILRTPVPLSYSRHNSRFLIIWLTLLPFTLWDQCHWFTLPVTGLVAFLLLGIKEIGVVVEEPFSILPLERICDTIEANVWEIHSIHSAKAMKEQAEARAAGLPSDLMEASDLVSVVSELALAAGTGGTGTTAGNGNGAGNGNGSAAAAVVAANGAVVVKV

SEQ ID NO: 18 - protein, 480 aa, Chlorella variabilis
MLAHSRPGAGSRPGNGAAKAGAGGRSAPPLQPAPALTRRALVHPVPSPRPTPGLVRQRPQRQRPQELPGATPEQQQQFPGSELDAAAKAEAAAAKLQRYPWHSDEWKEEYRKQVRVTFDFERWRTHRSSSRFWRHMAGLAQSNTAQGLAQPLAYVMAVSLGVAAYHVAAAAGWLPLWPVLKLAANAPFGLTSFALSLLLVFRTNSSYGRWDEARKMWGLVVNRSRDLTRQALGYIPAHQAELQSMLCRWVVAYSRCLMCHLREGEDLEAELRGVLLPEEVAELLAAEHRPNYCCQVLTEVLREAQLPAAVTSPTDSTGCVPAGAAYRMDENLTVFEDVTGGCERLLRTPIPLAYTRHTSRFMMAWLTILPFALWDTCGWAMLPVMAIVAFVLLGIEEIGVSIEEPFSILPLETICRTIESNVRESVGAHCQRQARRAQQAQHGGVPGDLPKRHAHSNGNNAVPALLFMPPQPVASAGQQH

SEQ ID NO: 19 - protein, 318 aa, Chlorella variabilis
VFTFERWNKHRNSMRYAKHLAHMFTSRVFRQLLGPVLAVMTIALAVGVYETLVGAGALPGHWPHVTLALGQGFNLTAFALSLLLVFRTNSSYDRWWEARKLWGGVVNRCRDIVRQVGPQGLVFFRDEDAHLKELLARWTMAFPRVLMCHLREDMDVGKEVAHILTAHEVAVMCAAAHRPNFVLQVMAETVRAARPNELCRMRMDDNLTFFEDAMGSCERILRTPIPLSYTRHTSRFLLVWLILLPFTLWAAYSWFSILLSGIFAFLMFGIDEIGVQIEEPFGWVAATYTAWHLACEKPGCCCWVVLCGGVRASAAHQR

SEQ ID NO: 20 - protein, 433 aa, Chlorella variabilis
MAILLGARVQPVAVAATPAAKRQARGSTLRHCQPAGLAALRQRCATSRCSRAALRTLAVASTDKTEPEHPYFSDDWKEYNRRFQRPVFDFERWKTHRSSSRYLRHVLGIFDSKIVQGLAKPLAYVMTLATGVALYHTLAEAGYLTDVPDLKATNAPFGLTSFALSLLLVFRTNTSYQRWDEARKMWGSMVNRSRDFTRQALGYVPESQPELRSMLVRWSIAYPRALMCHLRPGENIEEEVKDILKPEEVKALAASTHRPNYCMQVLTACLKQAQLPAAVTSNRDSYGAVPAGAAYRMDENLTVYSDVTGGCERILRTPVPLSYTRHTSRFMMIWLTLLPFTLWDNCGWAMLPITFIVSFLLLGIEEIGVSIEEPFTILPLETIARTIEANLRELEATHGPATLGKAGPGQVNATELLYDMIPSAANGRISINA

SEQ ID NO: 21 - protein, 503 aa, Chlorella variabilis
MQLGAGRQVSAAPVAGPASRLPCGLGLASPRHPWPASRGPLTHHQRRRQLRVAVAGGPGQQPEEAPERPAFPAAAAAVRRQQIQRERAAIRNRIENLVATDFDEEDAALIDLVGDEFKEADRKNLRTVFDFDRWKKHRSGSRYMRHARATFTSRITQGLAAPLLYVTGLSIAVASWHTAAEVRWAGPMGILSPIPELKIDTTFSLTSFALSLLLAYRTNAGYGRWDEARKMWGLVVNRSRDMTRQVGARLAGAGQCVHNIFITVCTCSRSLMCHLRGGEDIESELAGVLTPRELDALKASAHRPNYCVQVRGRRVARGSAVGGSLPPLPPAAASPQDVTGGCERILRTPIPLSYTRHTSRFMMIWLTVMPFTLWDAAGWAMVPLAMTVAFLLLGIEEIGVMIEEPFSVLPLEVISRTIEGNVRELERAHGLEAAAMKEREAALAAARDGGGAAAPAAPADGQVDALDLVLSILPEEELRQPWGPGLAMLSGTAGPATVPAGNS

SEQ ID NO: 22 - protein, 444 aa, Chlorella variabilis
MGAAAPPSAALARPAAGQRPLAAQLRQRQPGLAALTQRHHAAACRCNRCGRGVALRTSAVANTDKREQPHPYFTDEWKEYNRQFQRPVFDFERWKTHRSSSRYLRHVLGMGDSKIVQGLAKPLAYVMTLATGVALYHTLAEAGYLTDVPDLKATNAPFGLTSFALSLLLVFRTNTSYQRWDEARKMWGSMVNRSRDFTRQALGYVPYSQPELRSMLVRWSIAYPRALMCHLRPGENIEEEVKDILKPEEVKALAASTHRPNYCMQVLTACIKQAQLPAAVTSNRDSYGAVPAGAAYRMDENLTVYSDVTGGCERILRTPVPLSYTRHTSRFMMIWLTLLPFTLWDNCGWAMLPITFIVSFLLLGIEEIGVSIEEPFTILPLETIARTIEANLRELEATHGPATLGKAPEGAMNATELLYEVIPSAAPADTVNANSNGAQIAVKA

SEQ ID NO: 23 - protein, 273 aa, Chlorella variabilis
STFTFRRWAFHRSTSRYVRHMSGIFQSRIVRGLAQPLLSSCATATIVCVYEQALQDGWLPSFLPTFIMPSLPFDITASSLGLLLVFRTNSSYDRWQQAVSAWGDIETRARDTLRQLLAAASHSSAGHGGARQAALWLVAFSRSLKAQLTEDSDVQAELREVLTGQELALLLGAQHRPSFALAVLSELAEAAPLRDAQRVRVDENLSCFQDAAGRCERILRTPIPLSYTRHSSRFMVIWLSALPLGLWSQCGFGTIPLTVISPSCCWALRRLGV

SEQ ID NO: 24 - protein, 668 aa, Chlamydomonas reinhardtii
MQCQLKHGARPQSQRPNWLPARAATLRPAVQHGVRRGLTLGVKAAAAPLEDKKMPADMTTRQYRRVVYDFALWAKHRDVNRYLYNLRTIPGSRIIRQLSQPMGVVLAWAALFGFYETCLEAGVLPSYLPKMTLMSAEPQGLTSFALSLLLVFRTNSSYGRFDEARKIWGGILNRARNIANQAVTFIPAEDQAGREAVGKWTVGFTRALQAHLQEDIDLRKELEKATPRWSKEEIDMLVNAQHRPIKAISVLSELTRQLSITQFQALQMQENCTFFYDALGGCERLLRTPIPVSYTRHTARFLTIWLAMLPLGLWERYHWSMLPVIALIGFLLLGIDEIGISIEEPFGILPLDAICGRAQTDVNSLLKEDPAVMKYVDDVRSGRVKSPPPLPPAPAAPAAAAAAAAAAARSVSPQPDVAKTLGSLFTNVRAGVGAVAPGAPLMPQAPVRSPSPTRSVSPSFPRASAGTGMPPPVGMNGATPRVAAAPPTPPPVSRPAAPAAAPAAGSGFTMPNFSASLSGLTGGAAAAAKSAADAASSKLTKMADSMSSGAAAPAPPAAPARPSTSPRPSASSPISSSADADRSDSSRRPVNWRDELQSLKATREPNGNGNGSGVAPAAGRADADEEALRRFGNLAGRSRSGNGGGGSSDTELSEANRPRTRPDWRNQL

SEQ ID NO: 25 - protein, 381 aa, Chlamydomonas reinhardtii
MNNLRKKRFEEKTVERVYEADVYPAVFDFEAWHNHRSRKRYLEHCITLFRSYFFRDLLGPLAVLVGTAVAVGCYEQALQDHLLPGALPSFSGVSDTPFQLTSFALSLMLVFRTNSSYARWLDARQQWGLIVNTSRTFIRQVMTTLPESSCSELRDAIARWTIAFVRLSKLHLREHGDVRLEMAGVLRGEELPLVAAAAHRPLAAAHVLSELLRSAEAAGRLSDQSRARLEADVNMLSQALGACEKILRNPIPLSYTRHTSRFLVLWLLWLPIALWGKVSWGVVPVEAILCYLLLGIDEIAIQMEEPFGILPLENFCDAVQQSVEQVASMDHGVQDVVASYVCHIVQQDSGNGTGASEPAPPAEDGGNGFANIFFRMPADRC

SEQ ID NO: 26 - protein, 401 aa, Chlamydomonas reinhardtii
MGSGPRTIGLDSPTTTIRSAARDQRQLYEVEGRGTVPFDHYKEGSRHARRMTFTFDDWKRHRSSNRYLYHLKTLTESGIVRGIWAPVAWVTLFTAVVATLNVAHGAAMLPPWVPAMPQIAIEPVQLTSIALSLLLVFRTNASYSRWDEGRRSFGSITTVSRDIARQAFAWFRPDDYESRVRVGRWLVALGRSTMVHLREEHDMEDELREVLKPAEVQAVVSAVHAPSFCLQMITLIIRTAGLPQELVIRMDENVSRLTDAVSACERILNTPIPLSYTRHTARFLMAWLACLPFCLWTYCGPAMVPIAALVAFVLLGIEEIGVYIEEPFSILALEKLVNKLENIVNAMLRDSQELDRLVKVGSPTASPRPEPASTNSNNAVTDTAFGLGAAGGFSPAVAGAM

SEQ ID NO: 27 - protein, 438 aa, Chlamydomonas reinhardtii; identical to SEQ ID NO: 1

SEQ ID NO: 28 - protein, 450 aa, Chlamydomonas reinhardtii; identical to SEQ ID NO: 2

SEQ ID NO: 29 - protein, 466 aa, Chlamydomonas reinhardtii; identical to SEQ ID NO: 3

SEQ ID NO: 30 - protein, 456 aa, Chrysochromulina sp.
  source 1..456: note = CCMP291
MREHPLSYEEYMRQRSAGRDPLAEAVQGQSASMERGVVPPPPVKATPERTEAAFTPPAEFDFFQDVFKPTVESVVKAVVSPGAQQDSDESYMRVPWWEQGSTYSEDQRKDRRTVFMHDDWKRHRSSERFFRNIKTWPSSGINQALRKELTFVTSVSVFVVLANMLLYQYQDFGGVVHPGPLSFLDGPIKSLSLPALPFSMASPVLSLLLVFRTNTAYFRWNEARTLWGGLINNCRNIVRQTTTMFPNDAYHNALKKRLATETATFIKSLRNFLRGPEDDATLRKELYAYVNQGLMTSAQAEATLAAKNRPMFALAAMSATLRKANIDEMYISRMDSTISVLVDLTGANERIFKSPIPLVYTRLTARFLSVFLTLLPLAMWAALGESWNHWATIPATFILSVFLFGVEEVGIQIEEPFSILPLEAMCNGAIEAVQLEMLAAEQSQVFEAAGDAVAVA

SEQ ID NO: 31 - protein, 415 aa, Fistulifera solaris
MRFTTRQLIVGIALTASLHGAFAFVTPSVVGSSKASKSALDVSTVPRNDGSGLSYGERSRPFRRNFYTQPDWEKHRSRNRFKGNLLSITRSGVIRQLTNEVLFITAVATAVWAINCLTAAGYDDLNGVHHEGLLPFFPTLQMPSEFFTFSSPALSLLLVFKTNTSYGRWDEARKAWGVIVNSSRTILRQGAAWLQETNIPERDKIRLIARLAAAVWCFPRSMTRHVLSAREDDAAYAEDCRANLRPDLAEDLIAARHKPTRATYEISCAINDLPLSDIQKATLEYSVNQLCDAMGANDRILSSPVPLVYTRHLARFLEVWLLMLPLGLWPDFTNSWNHIAMIPITVLLSYFLLGIEELGVQLEEPFSVLPLHKITSGIGLSAEEHVQWFLNDSAKSEMKTNSAGSTSPNYQSLSP

SEQ ID NO: 32 - protein, 430 aa, Fistulifera solaris
MMLRLSNAVFALIALASVSQGSAFAPSSTSSRVDTALFGNTQSPPPIPMPKAISYGEESRKYRRTVFTHDDWVKHRSPERFVRNLSSIWSSGVYKNLANEVMATTGVATFVVLYNALVGGYTDFSGVAHAAPIQGLMQVGLPLAPFTLSSPSLGLLLVFRTNTSYQRWDEARKNWGMNINHTRDLVRMGTAMYDRQGVSPEKVEKDLKTLSLCTWAFVRAMKRHLSPESEDEEAFKRELYEKLPSAQAEAIIKAAHRPNRALFDLSLAIENLNMHFMRKNEIHKAATIFEDNLGSSERLLTSPVPLFYSRHLARFLSLWLLLIPFALYDTLGGTWNHVGLIPTTAVLSTFLFGIEELAAQMEEPFTILPMQAFCDKIGNWCNEIVSWSPGDNGMEVTIPSAVEQDHVVEEMMVANGSTRPSLRKVLGLKK

SEQ ID NO: 33 - protein, 430 aa, Fistulifera solaris
MMLRLSSAVFALIALASVSQGSAFTPSSTSSRVDTALFGNTQSPPPIPMPKAISYGEESRKYRRTVFTHDDWVKHRSPERFVRNLSSIWSSGVYKNLANEVMATTGVATFVVIYNALVGGYTDFSGVAHAAPIQGLLQVGLPLAPFTLSSPSLGLLLVFRTNTSYQRWDEARKNWGMNINHTRDLVRMGTAMYDRQGVPPEKVEKDLKMLSLCTWAFVRAMKRHLSPESEDEEAFKRELYEKLPSAQAEAIIKAAHRPNRALFDLSLAIENLNMHFMRKNEIHKAATIFEDNLGSSERLLTSPVPLFYSRHLARFLSLWLLLIPFALYDPLAGTWNHVGLIPTTAMLSVFLFGIEELAAQMEEPFTILPMQAFCDKIGNWCNEIVSWSPGDNGMEVTIPSAVEEDHVVEEMMVANGSTRPSLRKVLGLKK

SEQ ID NO: 34 - protein, 505 aa, Fistulifera solaris
MVRFTCIFPLVAALISNASAFVTPANSVAKHATPRFEGNERRLISAPSDGSVAKRSTSRFSSEGTGWDSFKGLRDFTNIPSGEEQRKFRRTVYTHKDWKKHRSQDRFVFYLLAMFKSGVYKNIGREVITITAVAAFVCIYNSLVGGFTDLNGVQQAALLQSQFLPKLTLPLSLFTITSSSLGLLLVFRTNTSYQRWDEARKNWGMNINHTRDLVRMANAYYDSTGVPPEVRADDMNHVALCTWAFVRCMKRHLSPEEEDEADFQKEILEKLPRQQAEMIIAAAHRPNRALQDLSYAIDDLPMHFIRKNEIHRAVTIFEDNLGSSERLLSSPVPVFYSRHLARFLAVWLLFVPFALYDSFNASWNHIAMVPATAVMSIFLVGIEELGTQLEEPFTILPMQGFCDKIYNWCMEIASWQPGDNGRPMRPVKPEHAYFTSTSEGVNGYANNYYTPQQSYYNEPAQPQQSYYSEPTPAPQYYNEPTPAQPAANQGWVDMAEFLRRQGSGV

SEQ ID NO: 35 - protein, 502 aa, Fistulifera solaris
MVRMICILPLVAALIGSANALVAPANNVAKQAMPRFSNNVAKSAVPRLLTSEGTRLFSSEGTGWDSFKGVRDFTNIPSGEEQRKFRRTVYTHKDWKKHRSQDRFIYYLLAIFKSGVYKNIGREVITITAVAALVCVYNSLVGGYTDLNGVQQAALLQSQFLPKLTLPLSLFTITSSSLGLLLVFRTNTSYQRWDEARKNWGMNINHTRDLVRMANAYYDSTGVPPEVRADDMNHIALCTWAFVRCMKRHLSPEEEDELDFQKEILEKLPRKQAEMIIAAAHRPNRALQDLSYAIDDLPMHFMRKNEIHRAVTIFEDNLGSSERLLSSPVPVFYSRHLARFLAVWLLFVPFALYDSFNASWNHIAMVPATAVMSIFLVGIEELGTQLEEPFTILPMQGFCDKIYNWCMEIASWQPGDNGRSMRPVKPEHAYFTSTSEGVNGYSNNYYTPQQSYYNEPAQPQQSYYSEPTPAPQYYNEPTPAQPAGNQGWVDMAEFLRRQGSGV

SEQ ID NO: 36 - protein, 433 aa, Fragilariopsis cylindrus
  source 1..433: note = CCMP1102
MGLASTLIEEVVEEEVWVPPSSFSTPDQYNSSPTLSAQSPTISVTLPVGLRPPKFGPKKDITYGENSRKFRRTVYTHEDWVRHRSPDRFFRYIASVPTSGIAKNLARELYFLTAITAIVVLYNGLITGYQDINGIAHGPVWSSPFLPPLTLPMQPFALASPSLGLLLVFRSNTAYQRWDEARKNWGMNINHTRDLNRMANAYYDKTGISDEKRKDDLERLSLCTWAFVRAMKRHLSPADEDEAAFKAELYEKLPKLQAQQIIKAAHRPNRALYDLSLAIENLPMHFLRKNKLHAALTIFEDTLGSSERLLTSPVPLIYNRHTSRFLLVWLLLLPFALWEEFGFMWNHIGMIPGMSILGLLLFGIEELATQLEEPFTILPMQAFCDKIWNWCNEIASWEDGQNGITTNRSYQGRTEVYVDNSYENENAYTTPTP

SEQ ID NO: 37 - protein, 430 aa, Fragilariopsis cylindrus
  source 1..430: note = CCMP1102
MRFVGVSSLMSMAAVMTVVVVSCHVGSASAFVPPSGKTSSSWNSVASQQAKDPLHILPPDATDIPYGEESRKFRRTVYTHDDWIKHRSPDRFFKTLRTTTTSGIYKNVGREVLAVTFIATFVMLWNMLTGEYTDLVGLVHPGIMRGQFTPSLALPMSPFTLSSGFLGLLLVFRTNKAYQRWDEARKNWGMNINHTRDLVRMGTTFYDDAQISNPEQRDIDLERLSLCTWAFVRSMKRHLSPEWEDEQEFKMELYQRLPSQQAQTILDAAHRPNRALYDLSLAIEQLPMHFIRKDQMHKALTIFEDNLGSSERLLTSPVPIFYNRHVARFLFVWLAFLPFALYDPFKESWNHVMMIPTTALLSLFLFGIEELATQMEEPFTILPMQAFCDKIGNWCNEIVSWKDGDNGVYNKPSNNFPAKGHNSVYFESEL

SEQ ID NO: 38 - protein, 393 aa, Fragilariopsis cylindrus
  source 1..393: note = CCMP1102
MCFAFCERLLDVMSSSCDTAMLLEKYQVTTKDIPYGETSRQFRRTVYTHDDWVKHRSPNRFIKNLSTIVSSGVYKNVGREVASTTSIATFVVLWNMLTVGYDDLNQIHHLPLINSYYVPGLTLPLQPFTLASGSLGLLLVFRTNTAYQRWDEARKNWGMNINHTRDLVRMGNTFYDRTGVSDETRKEDLQRLSVCTWSFVRCMKRHLSPEWEDEAAFRLEMFERLPESQANAVINAAHRPNRALFDLSMAIENLPMHFMRKNEMHKALTIFEDNLGSSERLLTSPVPLIYNRHTLRFLSIWLLLMPFSLYKPFDSTWNHIGMIPATAMISLFLFGIEELATQMEEPFTILPMQAFCDKIGHSEVYFENLGSSPGVVDGSNAFVEQQPEIVRVP

SEQ ID NO: 39 - protein, 467 aa, Gonium pectorale
MLRPLPGCVTARRPSASCPALWSVEAVLSTLRKKRLEPNTVERVYEADVYPAVFDFAAWTQHRSRGRYLEHCITLFRSYFFRDLLGPLLVLVGAAVAVGLYETFLQEGLLPEWVPDFGGVSDTPFQLTSFALSLMLVFRTNSSYARWLDARQQWGLIVNTARTFVRQAMTTLPEDSCSELRDAVARWTIAFVRLSKLHLREHGDVRAEMQGVLRGEELPLVAAAAHRPLAACHVLSQLMAAAAGAGAISDQSRQRLEADINGLSQALGACEKILRNPIPLSYTRHTSRFLILWLLWLPVALWGKAGWCVVPVEAILTYLLLGIDEIAIQMEEPFGILPLENFCDAVQQSVEQVTRMDHSVHDVVSSYVCALVPAAPATAAAPPVAAPPATAMGGTASAVVAAAAAEESSPVAASVTATAAPGDLNRAARDQGSGMEGSGVGDGSGDGRTGQGFWQAIWTPNIPSDRC

SEQ ID NO: 40 - protein, 481 aa, Gonium pectorale
MQGRAGAAVEEGVSLPTPTRNPVFNPTRWSYHRSTSRYGRHLTTIFKSVTFRNLQAPLSFLTMMACLVVLYRTSVELEQLPDFFEGVTFTDVPFQLTSFALSLLLVFRTDAGYGRWCAAMDAWGTLRNASADLIRKASAWVDDGPHLRRLVRWVAAFPLCLMVELRHDQGDPAMEGALRAELARVLRPYELQQVLAAGPYHHQFCLSVLTALVEAAQLGESRETAMLEDLHGFNDAACECEKILRFPIPLTYTRHTSRFMLIYLSALPLALYDSCDWAVIPVTAVIAFLLLGIEDIGVQIEEPFSILPLPDICADLHMTAQRAVAQRDMVRCLADPAVCRVRDEDLEPTARRHAAAHGGPGTSYGEGRGHAATGHPGAGPDAWQGDEYVVEAGFGSGGVAGGGGVGAGAGAGASARSNGNGAGGGAGGGGGKGVGARGRFVESGKGPGLVAAISENGNGNGYNGNGNGYSGGTASGWPHRP

SEQ ID NO: 41 - protein, 308 aa, Gonium pectorale
MRVAWSGIVRGISAPVLFVTAFTAVVAALNAAHAASLLPAWVPAMPAIAIEPVQLTSIALSLLLVFRTNASYSRWDEGRRSFGSITTVSRDIARQAFAWFRPDDGHNRRRLGRWLVALGRSTMVHLREEHKMEDELKYVLEPQEVEAVLSSVHAPSFCLQMITWIIRHAGLPQELVIRMDENVSRLTDAVSACERILNTPIPLSYTRHTARFLMAWLVCLPFCLWPYCGPAMIPIAALIAFVLLGIEEIGVYIEEPFSILALEKLVNKLENIINAMLHEWQNLDELSRTAMGTANGSQPVSAAASATA

SEQ ID NO: 42 - protein, 447 aa, Gonium pectorale
MQLSGRQQALSARGVAPLPSRLCRKRLIIAANAAKDPNAPIQSNPLGTLSSQGNTMATPPRSEAARKYFRTVYDFPQWQEHRSQFRLMKRLFTIPQSHVIQNALPSISWVGVVSTALTLYMTAHDQHILPEGFPSLVPNASCSAFISNTSVALSLLLVFRTNSSYGRWDEARKMWGGLLNRSRDIMRQGATCFPDDQVEAKKALARWVVAFARALRIHFQPEVTIESELKNILTPAELEMLAKSQHRPVRAIHAISQIIQSVPMSSIHQMQMSNNLTFFHDVLGGCERLLRAPIPVSYTRHTARFLFTWLSLLPFALYGQCGAGVIPVSTGIAAVLCGIEEIGVQCEEPFGILPLDVICNRIQADVMATLKDDADTKTILAEAGLVSLLPSAASAMAHTSASSNGNGNGANGNGANGNGANGNGAAVAAPQVPVSASAGMTVQISPR

SEQ ID NO: 43 - protein, 1866 aa, Gonium pectorale
MEHTGPSLAGPPPPPPLASAPSVAVERSGSEMGSALPYRRRSTAPAVAAPPPPSVMVPAAAAGPAAATALARHQPQAAAAAGAAPFPGAAAPLPPALQASTSRVGRLLQERFRRCRSHQAFNWTPEEAPLSPPPPPPPLSPPLPAVQKWAASTVPKPGPAAAAAAAAAPPTPYLSPSPSTSPSPSPSPPPPQATAAASAASGADGDFMTAAVSELVERPFRLVVVANRLPVTCVQDAPGGEWQLKASSGGLVSALRGLGFRSSMWIGWPGVWLPPGSDRNRLTELLRRDGYEPVWLESKLMDLFYKGFCNSVLWQLFHYDPPSLEGWARSAEQQASSQAAQWGAYVEANGRFADAVLATHGSADMVWVHDYHLMLLPGILKRAVPKMRVGFFLHTPFPSSEVYRALPVREQVSGGGGGGESRLWILSGVLAADLVGFHTYDYARHFISACARILGLEGTPEGVETHGSLTRVGTFPIGIETCRFVRAAADPDVVRQVAALRTRFVGRKIVLGVDRLDPIKGLPHKLLAFEKLLEEQPYWRDKVVLVQIAVPSRTDVPEYQHLSSIVHEIVGRVNGLYGSLTHVPIHYLDTCLAFSQLVALYGATDVALVTSLRDGMNLVSYEFVACQGDGSGPEPPGVLLLSEFAGAAQTHTAQHWADTFLSELNDTHIEADLRGRAAAPQLDVRQVLEAYGSSRRRLFVLSYSAVLTAGGEAAAGPDPANVVVLMGGCEAGRMEALAGRQLAGAWLAAENGLLVRPPVPPEALDGDDDSGGAPAAAATTVPARQAAYSPSSSSGHPTASQPPQPPLPRGWRWLPGGPPASLPPSALPSASPSTSPSPRGLAAARCVLDYFCERTPGSFVEARQTSLVWNYKYTDAEFGSLQARDLLQHLRAGPIGNEPLEVVQSGRSVEIRPVGVTKGTSMMRLLGLLAGGGGGGPPSGGGGAAAASTPDSGGGRSASRGYGGFNGHGNGTSRNGGGGGSWRRVGAGRRRLSIDGGRPSVDGGRGTLLPFRAGGGAGGASGAPAVGGGSDGRGGSREVEQGSSGGDGGGGRDAATAAAAAAGFDFALVVCGHLTPRDEDMFILFRDDGNSGGGGCGASASAVRAPDASGPANGFAANGLVQNGGGGQAIPHAPAPASRPQVAEGLGANAGGGGGIGGTGGGTGGGDGGAPGNRRRCASFTVALGRRPSCASSSLGGGGQLVDLLQALAAGRAAAPSQGEPPGAAAGTAAPISDPNNRRRQGVVADAPPQPHSHERWQPKAGGTSAIRTQRRGSDGDLGAAGTAAGAAAGVTTGNGIAECTAGRRSARVHEPHTTVRQASEPSEGESPFTADGAGGGGRSGSDADADADAPDANDGDGDGGDGDGPLRPLAEPLSAFPSPVLPAAASPLAFGGTPLQAFARWAAAPAAAGAAAVHSGSSNSGGGRSGGRGFGSGAAGGGGGGGCVARRSLVVVANAAKDPNAPIQSNPLGTLSSQSGTVSVLPRSEEARRYWRTVYDFPQWQKHRSSYRFAERLFQLSQSHILQNALPSISWVTLVATAVAAYGTAYDAEMLPEILPSIAPNASCTAFISNTSVALSLLLVFRTNSSYGRWDEARKMWGGLLNRSRDIMRQGATCFPDDQVEAKKALARWVVAFARALRIHFQPEVTLESELKNILTPAELEMLAKSQHRPVRAIHAISQIIQSVPMSSIHQMQMSNNLTFYHDVLGGCERLLRAPIPVSYTRHTARFLFAWLTLLPFALYPTVGWGVVPVCTGIAAVLCGIEEIGVQCEEPFGILPLDVICNRIQADVMATLKDDADTKTVLAEAGLLNLVPSAAAAVPVASAAPAAPAAAQQPAMAGSASSSSAGRGPSNGLQVRVAIGSQDKQ

SEQ ID NO: 44 - protein, 381 aa, Klebsormidium nitens
MNAPKIDPEPLLEYLQDETKKTIFDFVRWATHRNTRRYARHVSGLWRSGIIRSLRSPLLFVFFSSLSLVLYHTAKDRAWISPSFPTLSLSSTQPFTLSSLALSLLLVFRTNASYARFDEARTILGAILCRTRHLARQGLAWIDPKDADLIALLERWTIAFTFALAVHLRPDGDVRKELQGVLLPSELAALEEAEHKPSFVLSVLQHILPQAGVSDYLQGCMNESLAQLETSLGSCERIFNTPIPLMYTRHTSRFLMLWIGIIPFMMWEACGFASLILSPAIAFFLLGIEEIGVQIEEPFSILPITYIASVAERNIREMVKKRELTRGIVGRGRRGVSWEGGLGGPLDGVMNGCNAVSGQQSEGHFERAQKRCLFRTHFSSL

SEQ ID NO: 45 - protein, 460 aa, Klebsormidium nitens
MATSAHVAMRPQALWTGGGPASARSAPAFNPLPAQVGCPLATKEKWQRMWGHASLRASLDLADSKEEAARKTRTPSLPQRRSVRCQSAPKSNSPPPVALKHYLTPISADVKKEESMVYRRTVFGHPEWLRHRSSTRHYRHILSISSSRVILALGPPVLALTGGALAMALYNEAIVHDILPHELPLLKASALPLQLTAPALALLLVFRTNASYSRFDEARKMWGATLNRVRDLARQALSYMDDNEEREEVLRYLMAFPYAFKNHLLREDHIVDDMRNVGLNSDEIEILTSTEHRPNYCLQVMSQVISKNTDLTDFQRVNLDHNLTHFHDQVGGCERIFKTPLPLSYTRLTSRFLVLWHFFLPLALWDACHWMVVPATFLSAAALFCIEEVGVLIEEPFSILPLHAICNTAEKNVRELTRLQGEHLNKRAIGGKKQAQLNGASQNGVADGAAARRTNVQVTN

SEQ ID NO: 46 - protein, 428 aa, Marchantia polymorpha
METLCSIPKLTSRCLSFSTSLLELSRFHSFCPGLPQELNVRRGRTAFRLRAQPCSGISTIHVKESRVSRRQTIRCVGSGPEPEPYDPDFPKEEVFLYRRTVYDHKDWSRHRSSLRHSRHILSMRSSRVILALWPPVFGLTTVSIALSAYNECILSHWLPSFLPLLHVSATPFQLMAPALALLLVFRTNASYARFDEARRAWGSNVNRTRDITRQALTWMQHPSDAEKVKKLIRHCVAFNVCMKHHLVRGGDLRDDLHSWIDKEEIDGILASTHRPNYVLQVMSEIIHSCSITEMQLTRMDVNMTQFADNLGACERIFKTPIPLSYTRLTSRFLVLWHIALPLALWDHCQWVSVPATFLSAGALFCIEEVGVLIEEPFQIFPLDNICSTIKKNVDGLILAHTEIHHCHKHPPKGCDIPKKKSSGDAPKS

SEQ ID NO: 47 - protein, 337 aa, Monoraphidium neglectum
MGAAAAVVAYEALRTGALTFLPSLHVSPQLISLTSFALSLLLVFRTNASYERWDGARKTWGLIINRSRDIVRQGLTHIPRDKPDLRAMLCRWVPAFSKALMCHLRRDKSLRQELQGVLLPAEVDEVMRSQHRPNYVLQVLAEIIRTGRVRAPAAIRMEENLTSFEDCLGGCERVLKTPIPLSYTRHTSRSLMIWLTLLPFALVSTCGLATIPLCGFVAFLLLGETIRIEEIGVSIEEPFSTLPLAAICGSVLTNVRELQATHAADTTGSSAADVNPLPLSASQLVTRYGVAAAEAEVRGMDVAVSGAAAAAAAIRAGVPGNHAVWPQIRAGLAAAQS

SEQ ID NO: 48 - protein, 430 aa, Monoraphidium neglectum
MQHAARLQHAAAAPQLMRPPTHQRALCATISAYVRSIEKGDDLAKNQLCHVKRASVRAKASAKDPRVFDHDNWDVHRSTTRHARHIAGLCRSQTVANLVRPLSYVMGVAVTVVAVEALRAAGCAPAFLPSLQVNSALFGLTSFALSLLLVFRTNASYERWDSARKMWGLVLNRSRDIVRQGLTHIPRDKPDLRAMLCRWVPAFSKALMCHLRRDKNLSQELQGVLLPAEVDEVMRAQHRPNYVLQVLAEIIRTGRVRATAAIRMEENLTSFEDCLGGCERILKTPIPLSYTRHTSRTMMIWLTLLPSALVSTCGLATIPLCGFVAFLLLGIEEIGVSIEEPFSILPLDAISGSVLTNVRELQATHDAAAGATGAADAAPLPLSASQLVTRYGVAAAEAQVRGMHVGVNGAAAAVAANGTAVRSTHAVWPQ

SEQ ID NO: 49 - protein, 318 aa, Monoraphidium neglectum
MTSPIHSYGRWDEARKMWGLVLNRSRDIARQGLSYIPDDRQDLRDMLCRWTPAFSRSLMTHLRKGEDLREELKDILRPHELDSLLMSTHRPNYCLQVLSQVVKDANAGTAATIRMDDNITAFEDCLGGCERILKTPVPLSYTRHTSRFLIIWLTMLPFTLYGSCGFATVPLCCVIAFLLLGIEEIGVSIEEPFSILALQAICDSALNNVRELQAQHDISKAKSDQYSAIELVSMAAAEAAGASATAAAAESHNGNGNGNGSRAPSPAFATAGVANGGNRFAVAPPGSNGTAASSDGAGSGGSNVWPRKGNYSSVSFRK

SEQ ID NO: 50 - protein, 363 aa, Monoraphidium neglectum
MELRCAPASAPGIAPNDTAAAAASFGGAPISHGGAAAANTTKGGREQSVGGTARPRSALGRLSRSKILNLGAPEFVKYVLDKDGHDRHETDITRRRFWHNVRRREAGMRGKQTSKFFSAAGSGVLAPADLVFTFDRWNKHRNSLRYVKHLTHVFTSRIFRNLRGPVVVVMAMAVLVGTYETLRTTGYLPGFPHLAHSALDEGFQLTAFALSLLLVFRTNSSYDRWWEARKIWGGVVNRSRDVVRQGLVFFADEDTHLKETLACWAMAFPKVLMCHLRDSMDVKKEVGHLLSPLELELLCSSAHRPNFILQVISETIRAAKPHELYRIRMDENLTFFHDALGSCERILRTPIPLSYTRHTSRFL

SEQ ID NO: 51 - protein, 406 aa, Monoraphidium neglectum
MMVCGLMAGRVQLTDLGRPVAGDYEDAIKEESRRYRRTVFDIQAWEGHRSTTRYGRHLVGILTSRVVWGLARPLLYVAAVAAAVVAYESLRQAGLLPAALPSVQLTSKEPFGLTSFALSLLLVFRTNSSYGRWDEARKMWGLVLNRSRDIVRQGLSYISPDCWQLRDMLCRWTPAFSRALMCHLRKGNDLRQELQDILLPHELDSLLKATHRPNYVLQVLSQIISSANLSVAATIRMDQNLTSFADCLGGCERILRTPIPLSYTRHTSRFLMIWLTLLPFSLFGSCGLSTIPLSVAIAFLLLGIEEIGVSIEAEPFSILALEVICDSVLSNVREIQSTHAANGNGGSSTGVSGNGNGSGAPPAQQRREQLSAAQLVGLARSGPQGQKEWPPVQAVNRYPVPQAAGV

SEQ ID NO: 52 - protein, 358 aa, Physcomitrella patens
MHRNAVYGHDEWAKHKSCWRHGRHLKTIFASRPIVATGPPVAFCTLIAVFVVIFNHSVLVGHFPVWVPVIQVASIPFALTSSVLSLLLVFRTNSSYNRFDEARKAWGSNVNRTRDLARQALTWIRSPADLPKLHCLLRHIKAYSYCLKDHLTQDNTLREELAKVLEPTELELVLSSKHRPNYVMQVMSELIKQCKVSEWESMSMDRNLTQFHDNVGACERLFKTPIPVAYTRLTSRVLSLWHISLPFALWNSCHWLTIPATFFSSAALFYIEEVGVLIEEPFWILALMSISDGICSALDGLVAAHQETRLVWGVHPQAVCKKDHVVITFENSRSCASDGFRPQHDYIDVAMTRVNSVS

SEQ ID NO: 53 - protein, 416 aa, Physcomitrella patens
MAPGQVHTDCLWMKYSCLRNTYSKLQALQMKDLDFRHWASQEFSLVTPEEQAVLGSGLEPQKIMHRNKVYGHLEWARHKSSWRHGRHIFSILSSGVIFAVFPPVLVCTLFGTFVTIFNHFVQNGHLPTWMPILHVASLPFTLTSSVLSLLLVFRTNSSYNRFEEARKFWGSNVNRTRDLVRQSLTWISQPGDSLILLSLLRHIKAYSFCLKDHLTEDETLRDDLVGIVEPHELESILSSPHRPNYILQVLSELINQCHISQWEKMSMDENITTFHDNVGACERILKTPIPIAYTLVTSRFLILWHSALPLALWNDCGWLTIPATFLTGMALFYIEEVGVVIEEPFWILPLGSICTGIVSAIDGLSAAHKTSNLLWSLHQPMTDHVVQIAFERSKTDGFSKTRDQIDVMLTRINSIA

SEQ ID NO: 54 - protein, 297 aa, Physcomitrella patens
MGLHKSSWRHGRHIQTAFSTGVISSIIPRVFFCTLISVLVTIFNHAVMEGVLPHWVPSLRVPTLPMSLTAPVLSLLLVFRTNSSYNRLDEARKAWGSNVNRTRDVSRQALSWICDPDDADKLQSLLRHIKAFSYCLKDHLTQENLLQEELARVLEPREVELVLKSSHRPNYVLHVMSDTIKHCRISKWESKSMDRNITQFHDNVGACERLFKTPIPVAYTRMISRFLSIWHFLLPLALWNSCRWLTIPVTFVSGVGLFCIEEVGVLIEDPSTSLLCCQSAMAFPQLWMASARLMKEL

SEQ ID NO: 55 - protein, 669 aa, Raphidocelis subcapitata
MLATAPCAAACTPSRRGGRCHRRLWTPPRTAAHGQHAAADGDKQQQPDQPPPPPATAAAAGSDPAPPSPAAAPPPTPPGGFTPYWPSNYRKRLEAATLERAHEADVTPTVFDFALWRRHRSRLRYVEHLVTLTGSYFVLDLAAPVIVLTAAAAAVGLYETVLQGGMLPSFLPNIAMAAEGPFNAASFAMSLMLAFKTNVSYARWNEARCLWGSLVNRSRNLVRQGLMLIPREHAALKPVLARWSIAFARALKTHLREDGDVAGELAAILEPHEVAVVAANLNRPLACLQAMSAVVRAARLDPIGTSRLDINLETFEDIMGACERILKTPIPLSYTRHTSRFLTLWLATLPFVLWNHIHWGVIPSTAVISFLLFGIDEIAIQLEEPFGILPLEAICDTIQRNIEELVNSEGAVALALDASMHEARARLAQEEELARMLEEQLPFNWGPRGLSQPHSPAAAPGTPGAAGGAGAAAAAAALIAANRPRPFQDFPAGQPATRIEPWREEPVGATAPPPVSEEGQPPQWRPGWGRGGDGFGQQPECPPNCSCRMAEPGAAGGQQHSSPALRREDADRLRRALGEVAARRAAQSEARAAPLEPVEMPWYADDVASDSLLGAAAAEADPREAGVVEFLLGRAHGANAVRHPGTPMGTHEEEEEGPAAAGPAPPAGG

SEQ ID NO: 56 - protein, 564 aa, Raphidocelis subcapitata
MRSSLAAPPAHASSPRGLRAAAPPRPRAGARGGRPAVVVAALSQEDDQIKEQSRKFRRTVFTFEDWEKHRSTSRYSRHLSGMFSSRVVWGLQRPLVYVCGVSLAVILYEKSLELLSREPFGLTSFALSLLLVFRTNVSYERWDSARKMWGLLLNRSRDLVRQGITYIPEDRPELRTLLVRWTPAFARALLCHLRKDGDWEGQLGLTLKPHEVEQVLKATHRPNYVLQVLSQIIREADLGVAPTTRMDENLTQFEDVLGGCERILKTPVPLSYTRHTSRFLMIWLTLLPFTLYSTVGLGTAPLCAIIAFLLLGIEEIGVSIEEPFSILALEAISDSSLNNSRELAAMHATDAAVPLPEGADPPPAYTSARDLIALASAAAVASAAADALAAPAHAPAPASHSAPAPQEPAAPAAPAAVAPAAAAPAPAAPAPTAPAHSAPVPKEPAAEPERVPVLAGVGAGRAPVHLNGKSGNGATAYEPAAAPARANGARPAAAASPKPAAAAAPAAASHAAPAAPAAAAAPQAAAAPQAAAPAPAPAAKPLPNTGRAGSLRRTGGYGLIRFRS

SEQ ID NO: 57 - protein, 513 aa, Raphidocelis subcapitata
MQCAIGNAAVQPLGRPLGVPRSRAAAAAAAAVRARSWPRLTALRAAPGDGPEPSAPSTAGSLQSLAERLRATATPGEVQSDLNYYGLANLEDARRSGDEAQDDLLWRTPVSELAARRRGLGLVAADLSTEQERKLRRAVFGFDRWAAHRSTSRYLRHLKGIVSSRTMRALLPPLAFFGAVAVAAGWYTLELAPQYGLPKVVLNDSSLEITSFALSLLLVFRTDSSYARWEQALNAWQSVRAESKNLARQACYFVEEPIRRAMLMRWTVAFPHALLSHVRCDAPLRAQLAGVLAPHEVEAVEAVGAASAPEFVLQVMSELVEQCRLGDSHRAEQLYFSIKALGGAVGTCDKLLRYPIPLAYSRHTSRFMCVWLSALPFALFSSGLGWGAVPVTLVTAYLLLAVDEIGVQIEEPFSILPLEDILFDIQSETAALGERQGAVAEILRAGGIECTPRTPFDDAACAACGALVSPAGSLDDGDDAGGGGAGAGAGEALPGLPLRPRAPGGRRVVRTSR

SEQ ID NO: 58 - protein, 469 aa, Raphidocelis subcapitata
MKLSAGPMQSRAAFAGQRLAVRPALRSRAAKRTPAVQTSCGAAPKGLPSSVTSYDDDIKEKSRKFRRTVFTFESWEGHRSTERYARHIISMFSSRVVRGLSKPLFYMVGVSSFVVAYETARLAGLLPLWAPSVQLNSKEVFGLTSFALSLLLVFRTNASYERWDGARKMWGLVLNRSRDIVRQGLTYFPSDRPELKDMLVRWTPAFSKSLMCHLRKDDDYREVLQDILLPHELDSIMMATHRPNYALQVLSEIVREANCGTAPTMRMDDNITSFEDCLGGCERILKTPVPLSYTRHTSRFLMIWLSLLPFALYGTCGVSTIPLCVIIAFLLLGIEEIGVSIEEPFSILALEAISDSALNNSRELKAQHDAEVRNGNQMSATDLIAFEAAARGRTPLSKLSGAPAAAPAPVPAAAPAPAAAPAPAPAPVPAAAPAYVAAAATNGSANGAAPNTWPRPRSPVPTSGVSFRK

SEQ ID NO: 59 - protein, 443 aa, Raphidocelis subcapitata
MALAAARPALCAGPSQLRRAGQPAAQAARPLLPLRRARAPLRVQASKANPYAHMNVDDAIKEQTRKTARTVFDFERWEAHRCSGRYFRHILGVFNSRIIFGLLRPLMLVILTATAVVMWESLRQAGVLPPVVPSVQLAWSKELFGLTSFALSLLLVFHTNAGYERWDGARKIWGLMLNRSRDIARQGLTYIPRDQPHLRAMLCRWVPAFSRTLMCHLRQDSDFGAHLKGVLYPHEIEAVLSSTHRPNYVLQVISEIVKSARLPLAARVKMDQNITTFHDDLGGCERILKTPIPLSYTRHTGRFLMIWLLLLPFTLVGGCGVTTIPLCATIGFLLMGIDEIGVAIEEPFSILPLEAIANSALVNIRELEAAHQADPEPDFSAAEDHHTRTALDLVSATATAGTAQRAAAAAATAASAAAVAAAAAAAAAAGAPRSWPSSPAFAN

SEQ ID NO: 60 - protein, 1146 aa, Tetrabaena socialis
MQALRLKGQRPGLRASLQPRRTVVRIVANAAKDPNAPIQSNPLGTLSSQSGTVAPLLRSEEARRYWRTVYDFPQWQKHRSSYRFMERLFQLLQSHILQNALPSISWVTLVATLVAAYGTSFDQGLLPDGLPNISPNASCTAFISNTSIALSLLLVFRTNSSYGRWDEARKMWGGLLNRSRDIMRQGATCFPDDQVEAKKALARWVVAYARALRIHFQPEVTLESELKNILTAPELEMLARSQHRPVRAIHSISQIIQSVPMSSIHQMQMSNNLTFFHDVLGGCERLLRAPIPVSYTRHTARFLFAWLTLLPFALYPTVGWGVVPVSTGIAAVLCGIEEIGVQCEEPFGILPLDVICNRIQADVMATLKDDQDTKIVLSEAGLLGLLPSAVATVPVAAAAAAAAAAAAAPANGNGNGNGNGNGAYAQPAGAVASKDMQVRVAIGAEKHQRVCAPRGLRAPALCGRRLRLVVSADAAKDPNAPIQSNPLGTLSSQSGLVSTTPRSEMARKYFRTVYDFPQWQKHRNQYRLMKRLITIPQSHIIQNALPSIAWVGLVSTALAVYMTALDQHLLPEGLPSFAPNASCSAFISNTSVALSLLLVFRTNTSYSRWDEARKMWGGLLNRSRDIMRQAATCFPDDQVEAKKALARWVVAYARALRIHFQPEVTLESELKNILTPAELEMLARSQHRPVRAIHSISQIIQSVPMSSVHQMQMSNNLTFFHAKDPNAPIQSNPLGTLSSQSGLVSTTPRSEMARKYFRTVYDFPQWQKHRNQYRLMKRLITIPQSHIIQNALPSIAWVGLVSTALAVYMTALDQHLLPEGLPSFAPNASCSAFISNTSVALSLLLVFRTNTSYSRWDEARKMWGGLLNRSRDIMRQAATCFPDDQVEAKKALARWVVAYARALRIHFQPEVTLESELKNILTPAELEMLARSQHRPVRAIHSISQIIQSVPMSSVHQMQMSNNLTFFHDVLGGCERLLRAPIPVSYTRHTARFLFAWLTLLPFALYGQLGYAVVPTCLGIAAVLCGIEEIGVQCEEPFGILPLDVICNRIQADVMATLKDDEDTKIVLAEAGLLNLLPRPAALALEPAVQYAAASNGNGNGASTNGNGASTNGNGASTNGNGNGKPALVTVGASSAAAMKVEITPR

SEQ ID NO: 61 - protein, 705 aa, Volvox carteri
  source 1..705: note = f. nagariensis
MQSQLQPRLQLQGTRLNWLPQRSCVQRRSLRVDATSGAAPPPPAGKELSNDMVTRQYRRTVYDFSLWAKHRDVNRYLYNLKTIPGSRIIRTLGQPMGIVLAWAAMFGFYETCLESGVLPSYFPKLTLMSAEPQGLTSFALSLLLVFRTNSSYGRFDEARKIWGGILNRARNIANQAVTFIPAEDVAGREAVGKWAVGFCRALQAHLQEDANLREELQKAQPRWSREEIDMLCSAQHSWQQLQSCVNAFWPIKAISMLSELTRQLPISQFQALQMQENVTFFYDALGGCERLLRTPIPVSYTRILPLDAICTRAQTDVVSLLKDDPAVVKYISDVRQGRIAPPTEPPVAGAAPVAAAPPPPPPASAGGGISRSGSPTAQQQPDVMKTVTSMLHNVKAGIGAVAPAPPRPPSPQPRARSPRAASPGGPSPFPRASAGTGGAAAAVPSPPPIKPLTSSSSSSSGAVSKDSNNSTATAKKPASAPAASSAGFSMGFSGLADGAAAAAKSASAAAAKFSKIADSVVAGTPAAPASEAKRETAAAAAMQAQPRNTPSSSSSTPSAAPANGSSDDDRSSSGRRTAAAVNWREELAALRAGREDAEEPASASASYDREFPSSSWSFSSASSAAVVQSGDAEDEARRRFGGLAGRGARSDTTTSAAAVMRGNGNGLSENGYGNGYGNDNGNGNGNTVEARGARPRTRPDWRNQL

SEQ ID NO: 62 - protein, 403 aa, Volvox carteri
  source 1..403: note = f. nagariensis
MSSSMMVCGRQQRLSMPRGLGSALPGSRLCRSRLIIVASAAKDPNAPIQSNPLGALSSQAGTVTPLPRSEEARKYFRTVYDFPQWQTHRSQFRLMKRLFTIPQSHVIQNALPSILWVGIVSSALAAYMTAHDQHILPEGFPSLSPNASCSAFISNTSVALSLLLVFRTNSSYGRWDEARKMWGGLLNRSRDIMRQGATCFPDDQVEAKKALARWVVAFARALRIHFQPEVSIESELKNILTPAELEMLAKSQHRPVRAIHAISQIIQSVPMSSIHQMQMSNNLTFFHDVLGGCERLLRAPIPVSYTRHTARFLFMWLTLLPFALYGQCGLGVVPVCTGIAAVLCGIEEIGVQCEEPFGILPLEVICNRIQADVMATLKDDADTKTVLAEAGLERPQPQPQRGE

SEQ ID NO: 63 - protein, 446 aa, Volvox carteri
  source 1..446: note = f. nagariensis
MQIGTPMRAAASQRPLGLRYSCLPRKLIRVVANAAKDPNAPIQSNPLGTLSSQSGTVNPLPRSEEARRYWRTVYDFPQWQKHRSPYRFMERLFQLSQSHILQNALPAISWVTLVATMVAAYGTSYDQHLLPDGFPSISPNASCSAFVSNTSVALSLLLVFRTNSSYGRWDEARKMWGGLLNRSRDIMRQGATCFPDDQVEAKKALARWVVAFARALRIHFQPEVTIESELKNILTPAELEMLAKSQHRPVRAIHAISQIIQSVPMSSIHQMQMSNNLTFFHDVLGGCERLLRAPIPVSYTRHTARFLFAWLTLLPFALYPSAGWGVVPVCTGIAAVLCGIEEIGVQCEEPFGILPLDVICNRIQADVMATLKDDADTKTVLAEAGLAATGAAVAAPRWLMRRAPRSGWEEGARELVVCRRARITSSSSDSECEVVASPCVRFLIEP

SEQ ID NO: 64 - protein, 398 aa, Volvox carteri
  source 1..398: note = f. nagariensis
MQADLAGFHARPTFKRTRNNCLRHAHRKPPFARPVAFGSPIQPLDRSSLWTSSALWTVESVLVNLRRKRFEPKTVERVYEADVYPAVFDFASWTQHRSRGRYLEHCITLFRSYFFRDLLGPLLVLVGAAMTVASYETALQTGLLPRWLPDFGGSSDTPFQLTSFALSLMLVFRTNSSYARWLDARQQWGLIVNTARTFVRQVMTTLPETSCCEVRSALARWTVAFVRLGKLHLREHGDVGAEVQGLLDGEEVPLVLAASHRPLAACHVMSGLLRSAEGSGQLSEQARMRLEADINLMSQALGACEKILRNPIPLSYTRHTSRFLILWLLWLPLALWGKVGWCVVPVEAVMTYLLLGIDEIAVQMEEPFGGSGLGGAGRSAASCRWKTCVRLYNNPWSN

SEQ ID NO: 65 - protein, 389 aa, Volvox carteri
  source 1..389: note = f. nagariensis
MSTDFVAYSVCTSALVCVTFTFDDWKRHRSSNRYLYHLKTLSESGIVRGIAAPVLFVTAFTAGMTLLYVVAALNVAHAARMLPAWLPPLPNIAIEPIQLTSIALSLLLHMLTGVRHESSETPPKPRHPRLLTCRRFPCAPLSLPVLFYDMEKVFRTNASYSRWDEGRRSFGSITTVSRDIARQAFAWFRTDDMTNRSRLGRWLVALGRVTMVHLREEHGMQEELKGVLKPQEIDAVTSSIHAPSFCLQMITWTIRTAGLPQELVIRMDENVSRLTDAVSACERILNTPIPLSYTRHTARFLMAWLVCLPFCLWSYCGLAMVPIAALVAFVLLGIEEIGVYIEEPFSILALEKLVNKLENIVNAMLRESQWQTQSFALHILFVGPFPVSV

SEQ ID NO: 66 - protein, 929 aa, Micractinium conductrix
MQRVLAASGSTTHMAARPGLSGSSRARAATAAATARLTPAGRGNALRLAMPQHAGAAAAGPRTRAPVRVSAAADPAVKLTGDDLKEANRKQMRTVFDFELWRKHRSSARYWRHITGIFESRIVSGLAAPLGYVAALSTTVALYHVLAEAGYIMDVPDLKLASNAPFGLTSFALSLLLVFRTNSSYGRWDEARKMWGLIVNRSRDFVRQGLGYIPDSQPELQNMLCRWTVAYSRSLMCHLRPGENLEEELKETLPAHELKALLSSTHRPNYVVQVLTAVLKEAQLPTAVTSSRDSTAVVPAGAAYRMDENLTVFADVTGGCERILRTPIPLSYTRHTSRFMMIWLTLLPFTLWDSCRWAMVPISLIVSFLLLGIEEIGVSIEEPFTILPLEVISRTIEGNVKELQKMHSSEGKAVMGASAEGDNLSAQALVDAVLPAISGNGNGGALSAAGSSSGSSRIVIKPAMAMQQRASAAPKRSAATGARALVAHPAACRCAAHRRGRSLVVRAASDNVMEVGGRTIVVEDDGTIIISQPAASAAVDVKESAEVEYLTARSAIVNKHFEGSLGADDFIQRVEMALYAFGFTGENSIAMVNLCRDEVTVTLKQKIDAVFGASFSTNGLGGVLTCGAVGMGAGFSHSPICDTTGKERYIFFSFPHISINSKGEVGPMSRPGRPGQSCACGALIKSWTELRSEGVSCNCKIPGVHDAENPEYSILKQRIARRLRHEGETDESVQQLSLVDITKVAERTISDDLEYLISKTVDTNKADYAVITGVQVHNWAFDFEDDSPNLEFVFPTSAYVVVDGVKTHVDLAAMPPLTPRQIRLVHGGDQGVVCNTGGQSTLRAEDPPYAYDSKDARKVQRTRLQNYISLIKDEGLESVAAAPSSPAWAKQITKSVADRTNSTALDMKFAQNEDLKEMQAQLEAKYQSL

SEQ ID NO: 67 - protein, 250 aa, Chlamydomonas reinhardtii
MTRWRPRRPWPAGLVAFARALRIHFQPEVTIESELKNILTPAELQMLAKSQHRPVRAIHAISQIIQSVPMSSIHQQQMSNNLTFFHDVLGGCERLLRAPIPVSYTRHTARFLFAWLTLLPFALYGSCGVSVIPVCSGIAAVLCGIEEIGVQCEEPFGILPLDVICNRIQADVMATLKDDADTKTILAEAGLISLRANSAMAVENALPDLDSINAAAPNGNGSHNGNGAAVPVSVSAGASGNGMNVRISPR

SEQ ID NO: 68 - protein, 883 aa, Micractinium conductrix
MRRRALAAAAAAGSTAPPPHRLGDDGAGNAAPPPLDPSMPLPLPWQAYFDGRHAASCPGRGATFNVYTAGSAGPVVLCLHGGGYTGLSWSLIARALKDKHRVVAPDLRCHGLTVAEDEADFSADTLAGDVVALWRALFAGGGAAGSDGSTGSGGGGGGSSEADVQQHLQRLRQGPPPPTVLVGHSMGGAIAVHAAALGGITSLAGIVVIDVVEGTALASLPYMAGVLEKRPRRFASLQQAVDWAVDSGMCKRQEAAHVSLPAMLRREPPGGPTGGGAEAAGGVVRLVPGGLAPLAEEGEEEEDGAEAAAAQQQQQQQQQQQQQQVQQPAAGTQQAAPSGGGGGGGGWVWRTPLERSAPFWEGWYQGLSDMFLKLSVPKVLVLVGTDRLDRPLTIGQMQGRFQPVLLPQAGHAVHEDEPEATADAIAAFIKRFRNGEPPLQIPRPTPGLRPVLPVAMGPADSLRVQSKPDRSIDRASSSDKVVSFPALDSIERYYEPVKEDARQYRRTVFDFEKWREHRSTKRYARHIKSIKGSRMVRGLAAPLLWLTAVAAAVATFNTLVEAGLAPDVLPELKFSNNGPFGLTSFALSLLLVFRTNASYARWLDARKNWGTLTNRSRDLTRQALTFFPAEDPALFHLLCRWVQAYSRSLMCHLRDDVSIEEELPKALLPYEVEAVLASRHRPLYCLQMLSEIVRAAHVPPAGQPPAGAAAAAVAAAADPFAGAALAAALRAAAAQRMDENLSIFEDVCGTCERILRAPIPLSYTRHTSRFMTIYLSLLPFTLWDSAGWAMVPLIAIVAFLLLGIEEIGVSIEEPFSILPLEQMCDTIQASVVELRAINSQQHEGSSGDGGNGNGVAAAPRQPPSARALLRRGVAEAAAADALA

SEQ ID NO: 69 - protein, 2445 aa, Chlorella sorokiniana
MGGHGFIEGFLKSWGVILASEIGDKTFFIAAIMAMRHARLTVFAGAMAALAAMTVLSAALGWAAPNLISKTYTHYAATALFFFFGGRMLYEAVTNAHAGENELDEVEKELEAVDKSPKTGGPAANGKNPLLGTLRRFVSPVLLEAFTLTFLAEWGDRSQIATIGLAAAADVFGVTVGGILGHAMCTGAAVLGGKHLAEHIDERKVAYFGGVLFLLFGAHSLYTGVPECGGEAKMPSLAATEPVDYAVLGAAGAAALAGLAACLYTVPWRLCRAAAPPSAPKATRSFNVLWHGRSLLALLLTAWALSPLLRVSRLWGANSFVFSDGVTSWTGSGWMCRIYLTAALGVLQPLCAFLALLMLTAGLSRRHRPEALRHPNARLLGLAALCTLPVAAVQVVVAWLSLAVQYQGQPLEQAPRSVLGYFFATYWEGSPEQCGTQAAAAAAVVPAAANDAAALQTPGGSGSLSLSAAGSPGDAAAAGAAVAPDGGCTLCCFPAAAVAVHAIFTVAFLAALWVTCSRLADAVLNSRLKRRMRLFQLAHSLLAVGGVAALGVSIVQGPFTWLNQACWAAYVGTAVATVALVLWEVVVSPVRDLRSVDKRLALWADATTAAFAAAGADEPPMSTQSSGGSSVLIKAHNRSAEAEAYVQAAAAARTLSASPAQGQEKGQPAPPRPSLQLAPLALPPLPQGTPRRSPAKQPTPLWRQQAQPSLLRQQYGTAPSPLVSPPRQPARVTVAGAAADLPPPGPVTAGLKAAQRRQPGSLRGKQEDVWSLEDAAEMGAACSNSEKELGRRYMRSLSTFNFQRWAFHRSTDRYARHMLGIFQSRIVRGLAAPLLSAGATATLVCLYEQALQQGALPAQLPSLVISPLPLDLTSAALGLLLVFRTNSSYERWQQALAAWGAITTRSRDTMRQLLCYTAATPEAGPASTKLLTVAATGRWLVAFARALKAQLTEDSDLRAELEGILTPTELDLLCTSYQPSSFTLSVLTELVAAAPLRDSQRIRVDENLTACEDAMSACERILRTPIPLSYTRHTSRFMVIWLSCLPLGLWPAVGWGTVPLTVAIAFLLLGIEEIGVAVEEPFSILPLDELCREIEISLSDIVEQASSSKRAAKEAVSAAAAAAVTAAAAEAAAAAAAAAAEAADGSGSSTSSGDSTPLYAAAAQAPAAANAVAPLLPGALGRASPLKMAAKDLESGDASTSSDGCRLEPSPKKGLKQSLSRAFLSSGLQVTFKDITYTVVNSQNKKEKLDLLKGVGGYLQAGEMAALMGPSGSGKSTLLDVLSGRKTVGELQGEVAFAGNRPTPQFLRRFTGYVEQFDTLLDILTVEEMLMYTAELKRPVSQTLEQKKGAVDELIEVLGLDGCRTVRIGNPMARGISGGQAKRVNIGIALVTNPRVLFLDEPTSGLDSYTANEVMSVVKSLASHGITVCATIHSPTPYTFNLFDRLLLLLKGRVVYFGPNGKLALNYFHTQCPCLAGLKEGENEAEWLVDLTTQADRQGRAADFADTYERSEFKAAAEKEIAAQLATASDLDEATKRDLAVRRDTVTPFWWGLRTLLKYRTLKNYRNPEYLGPRIGDKLIFSLLIFTLFWKVGNNLAQDNLMNIAAVLFMWTTLPAFGAASYVPAIVLERPLFTRERNDGLYRVITYLVAKIIEELGLALLCSIVFANIVWWALQLQGSFALFWLIYFLTLSTGIVLAYTVAALSPNMDVANAALPAYVVTLLFFAGFLIRFDKIPNYWKWYSYIDVLRYAWGALMKNQFNGDRNVEFVSGQTILDYYSLSGINAWGWLGIEAAFVAVFFGFAYLALKYISHPLKMTTKDLEAGDASTGSDGCHLGLEASPKKGLKQSLSRAFLSSGLQVTFKDITYTVVNSQNKKEKLDLLKGVGGYLQAGEMAALMGPSGSGKSTLLDVLSGRKTVGELQGEVAFAGNRPTPQFLRRFTGYVEQFDTLLDILTVEEMLMYTAELKRPVEQSLEHKRAAVDELIEVLALEGCRTVRIGNPMARGISGGQAKRVNIGIALVTNPRVLFLDEPTSGLDSYTANEVMSVVKSLASHGITVCATIHSPTPYTFNLFDRLLLLLKGRVVYFGPNGKLALDYFHTQCPCLAGLKEGENEAEWLVDLTTQADRQGRAADFADTYERSEFKAAADKEVAAQLATASNLDEATKRDLAVRRDTVTPFWWGLRTLLKYRTLKNYRNPEYLGPRIGDKLIFSLLIFTLFWKVGNNLAQDNLMNITSMLFMWTVLPAFGAASYVPAIVLERPLFTRERNDGLYRVITYLVAKIVEELGLALLCSIVFANIVWWALQLQGSFALFWLIYYLTLSTGIVLAYTVAALSPNMDVANAALPAYVVTLLFFAGCLIRFVDIPNYWKWYAYIDVLRYAWGALMKNQFDGDRNVEFVGGQTILDYYSLSGINAWGWLGIEAAFVAVFFCFAYLALKYISHVRR

SEQ ID NO: 70 - protein, 282 aa, Chlamydomonas reinhardtii
MATLKDDADTKTVLAEAGLISLIPGHWRNADTNLSLSRRYGGGYEVVYCAPPRSSQAGPVSWAANKDPNAPIQSNPLGSFSSQLQNQPTLPRSEEARKYFRTVYDFPQWQTHRNQYRLMKRLFSIPQSHVIQNALPSIMWVAFTSTCVAAYMYGYDQHMLPEGFPTLAPNAACSAFISNTSVALSLLLVFRTNSSYGRWDEARKMWGGLLNRSRDIMRQGATCFPDDQVEAKKALARWVVAFSRALRIHFQPESGYWQSARALLLAGRASVIQGSACVVATQ

SEQ ID NO: 71 - protein, 302 aa, Chlamydomonas reinhardtii
VYDFPQWQKHRSSYRFAERLFQLSQSHILQNALPAISWVTLVATLVASYGYSYDQHMLPDVFPSISPNASCTAFISNTSVALSLLLVFRTNSSYGRWDEARKMWGGLLNRSRDIMRQGATCFPDDQVEAKKALARWTVAFSRALRIHFQPEVTIESELQNILTPAELQMLAKSQHRPVRAIHAISQIIQSVPMSSIHQQQMSNNLTFFHDVLGGCERLLRAPIPVSYTRHTARFLFAWLTLLPFALYPTTGWGVVPVCTGIAAVLCGIEEIGVQCEEPFGILPLDVICNRIQADVMATLKDD

SEQ ID NO: 72 - protein, 414 aa, Chlamydomonas reinhardtii
MSVARRSAAPEIFILAAARALHLCSLKKNALSNMVIRFMSINPRSLSWLQSFCQKNAKSAARDQRQLYEVEGRGTVPFDHYKEGSRHARRMTFTFDDWKRHRSSNRYLYHLKTLTESGIVRGIWAPVAWVTLFTAVVATLNVAHGAAMLPPWVPAMPQIAIEPVQLTSIALSLLLVFRTNASYSRWDEGRRSFGSITTVSRDIARQAFAWFRPDDYESRVRVGRWLVALGRSTMVHLREEHDMEDELREVLKPAEVQAVVSAVHAPSFCLQMITLIIRTAGLPQELVIRMDENVSRLTDAVSACERILNTPIPLSYTRHTARFLMAWLACLPFCLWTYCGPAMVPIAALVAFVLLGIEEIGVYIEELDRLVKVGSPTASPRPEPASTNSNNAVTDTAFGLGAAGGFSPAVAGAM

SEQ ID NO: 73 - protein, 374 aa, Phaeodactylum tricornutum
  source 1..374: note = CCAP 1055/1
MLSSSRWGSAFVCFLALACSSSYRTQAFIVPSGTSSRVSVAVRSLPPPRDIAYGEESRKYRRTVYTHDDWVNHRSPDRFWRNIIAMPTSGVYKNLAKECIATTAVATAIVVYNALVGGYTDFGGVQHAAVLQNELLPKIGMPVSPFTVSGSFLGFLLIFRTNSSYKRWDEARKNWGMNINHTRDLVRMGTAFYDKTGVTDEQRKKDLQALSLATWSFVRAMKRHLSPEQEDEQDFRRELHERLPPRQAQAIIDAAHRPNRALFDLSVAIENLPMHFMRKNEIHNAATIFEDNLGSSERLLTSPIPLFYARHTARFLGVWLLLMPFCLYDPFAGSWNHVGMIPATALISIFLFGIEELATSMEEPFTILPMQAFC

SEQ ID NO: 74 - protein, 455 aa, Phaeodactylum tricornutum
  source 1..455: note = CCAP 1055/1
MMRNFASVLLLLSSGAAAFAPVQHNGVRTIATPSTPLYGNTKQPPALPPIKDISYGEESRKYRRTVYSHDDWVKHRSSDRFLRNLLAIGSSGVYKSLAKEVLATTGVATFIVLYNCLVGGYTDLEGIKHSALIESVWAPLMALPLAPFTLSSPSLGLLLVFRTNTSYQRWDEARKNWGMNINHTRDLVRMGTSFYDNAAVSSEQRAKDLKALSLATWSFVRAMKRHLSPESEDEQDFRRELFERLPAPQAQAIIDAAHRPNRALFDLSVAIENLPMHFLRKNQVHQAVTIFEDNLGSSERLLTSPVPLFYSRHTARFLSFWLLLLPFALWDPFAGTWNHVGMIPATAVISIFLFGIEELATQMEEPFTILPMQAFCDKIGNWCNEIVSWQAGDNGMAVNMPSMISPEGLPELKEPAPVPAMAVASVAAAMPVMANGDINGDTTGITMDQPHNAIP

SEQ ID NO: 75 - protein, 408 aa, Thalassiosira pseudonana
  source 1..408: note = CCMP1335
MPSFTSLSTLLLLALSSPQISAFAPLSSTSTPINVAPSTTTSTTNLQMGPPKTDIVLSETYGEGSRKYRRTVYTHNEWVKHRSSDRFAKNLFSMVNSGVYKSLAKEVFATTAVASAIVAWNGIAGGYTDFNGVEHGAIMSFLPQLVLPLTPFTLLSPSLGLLLVFRTNSSYGRWDEARKMWGLNINHTRDLNRMATAWYGHDNQIIDPAKRAEDLRQVSLYTWAFVRSMKRHLSPPSEDEEAFVEELYARMAPEQAEAIISAAHRPNRALYDLSVVIDKLPMHFMRKNEINKNLSIFEDTLGGCERLLSSPVPLFYTRHTARFLSTWLLLLPLAMYQPFSGSWNHVAMIPATALTSVFLFGIDELSTQLEEPFTILPMQGFCDKIGGWCDEIVSWRGQGLDKEEQQYY

SEQ ID NO: 76 - protein, 390 aa, Thalassiosira pseudonana
  source 1..390: note = CCMP1335
MGPPIDPSVPVTDQVGEGSRKYRRTVYTHDDWVRHRSPDRFGNNLSTLFNSGIYKQVANEVFATTAVATFVFLWNMIAGGYTDLAGVQHGPIIDSPLAQMVGLPMTAFTILTPSLGLLLVFRTNTSYGRWDEARKMWGLNINHTRDLNRMATAWYGNEGNMDSVAFMGGDIPYSQPIDPVQRAYDLGQVSLFTWAFVRSMKRHLSPPEEDEEDFKAELRARLTPEQAENIINAAHRPNRALFDLSVAIENLPMHFLRKNAINTNLSIFEDTLGGCERLLSSPVPLFYSRHTARFLSTWLLLLPFGLYEQFKDSWNHIAMIPATAFISVCLFGIEELATQLEEPFTILPMQGFCDKIGGWCDEIVSWAGQGQQEYTEENAMSNEQEMTYWR

SEQ ID NO: 77 - protein, 426 aa, Erythranthe guttata
MTNPIHHSATKLSPSSSSSSLSSHFTPKTLKKFHLYPSNNHHRSNHHLRFSCCQSSKDSNFSLNQDSTFFSILLALPNWVDSIQEKGLEKKRSLYDHGDWVQHRSSSRHVRHFLSSLGSRVILSLLPPVIVFTSVAVVVSTYNTAVALAWLPEFFPILRASSLPYQLTAPALALLLVFRTEASYSRFDEGKKAWTKVVSGTNDFARQVIASAVGPDDSVLKTAILQYVVAFPVALKCHVIHDSDIEQDLKNILETDDLAVVLTSNHRPRCIIEFISQSLRSLDVDPTKLQVLESKLSCFHEGIGVCEQLLGIPIPLSYTRLTSRFLVLWHFTLPIILWDDCHWIVVPATFISAASLFCIEEVGVLIEEPFPMLALDELCEKVRSNIEDVMRNEKLIQARVNAKRKTHYKRDSPNGWTASLENRKPD

SEQ ID NO: 78 - protein, 412 aa, Solanum lycopersicum
MTNPRTLFTIQSPTNASFSSHFTLKNPSKSQQQPPVYKKLNFRVLCSSQPSRTPQNSNLISTLVKILRVVPDWADKIQEGGMRKKRSLYKHETWVQHRSSLRHVRHLFSSFNSRVVLSLIPPVIAFTSFAFVIASYNSAVSFHWLPEFFPILRASPQPYQLTAPALALLLVFRTEASYSRFEAGKKAWTKVIAGTNDFARQVIACVDKRDDVLKEALLQYIMAFPVALKCHIVYDSDIASDLKNLLEADDLAVVLSSKHRPRCIIGFIAQSLQSLNLEGTILSQLESKISCFHEGIGVCEQLLGIPIPLSYTRLTSRFLVLWHLTLPIILWDDCHWIVVPATFISAASLFCIEEVGVLIEEPFPMLALDELCQRVHDNIHEAMANEKQIQEIVNSKRKRSFSEHSPNGWPTS

SEQ ID NO: 79 - protein, 414 aa, Fragaria vesca
  source 1..414: note = subsp. vesca
MLTQPTNCIKPTSNILFSFNSIPKSFFKLHPPLNPRTLSLKHHCSQYPNPTPSSSSSSSPFQPLSSILRIIPDWADEIKERGFRQDRTLYDHEKWVHHRSSYRHLRHFLTSLSSRVILSLIPPVIAFTLVAVVIASYNTAVALEWLPEVFPMLRSSTLPYQLTAPALALLLVFRTEASYSRFEEGRKAWTKVIAGTNDFARQIISGVENSGDAELKKALLQYVVAFPVALKCHVIYGSNMGRDLQNLLEVDDLLVVLSSKHGPGCIIEFISQNIRMLKLEESRRNMLESNMSCFHEGISVCENLIGTPIPLSYTRLTSRFLVLWHLTLPIILWDDCHWIVVPATFISAASLFCIEEVGVLIEEPFPMLALDDLCRSVQENVQEALSREKLIQARLTAKGRIQKQQHCPNGQPKS

SEQ ID NO: 80 - protein, 416 aa, Gossypium arboreum
MAQSPNSPKLSLSPSFTSKPFLHFHFNSSLPAKFKRPPSFKILFSASQPPPPSSKPTTAPKTFNLITLLRAIPDWADRIQERGMQQNRALYNHEKWVHHRSSLRHLRHLLSSLQSRVILSLVPPVLAFTSVATVVALYNTAVDLHWFPGFFPVLRASSLPYQLTAPALALLLVFRTEASYSRYEEGRKAWTKVIAGTNDFARQVIAGVDNSADDQSIKAALLQYIMAFPVALKCHVMYGSDIGRDLQNLLEVDDLAVILNSKHRPRCIIEFISQSLHLLNLEESKRTTLESKISCFHEGIGVCEQLMGIPIPLSYTRLTSRFLVLWHLTLPIILWDDCHWIVVPATFISAASLFCIEEVGVLIEEPFPMLALDELCIVTQNNIREAIATEKDIQAMLKGKRKRHSYKRSPNGCPNT

SEQ ID NO: 81 - protein, 416 aa, Gossypium hirsutum
MAQSPNSPKLSLSSSFTSKPFLHFHSNSSLPAKFKRPPSFKILFSASQPPPPSSKPTTAPKTFNLITLLRAIPDWADRIQERGMQQNRALYNHEKWVHHRSSLRHLRHLLSSLQSRVILSLVPPVLAFTSVAIVIALYNTAVDLHWFPGFFPVLRASSLPYQLTAPALALLLVFRTEASYSRYEEGRKAWTKVIAGTNDFARQVIAGVDNSADDQSIKAALLQYIMAFPVALKCHVMYGSDIGRDLQNLLEVDDLALILNSKHRPRCIIEFISQSLQLLNLEESKRTMLESKISCFHEGIGVCEQLMGIPIPLSYTRLTSRFLVLWHLTLPIILWDDCHWIVVPATFISAASLFCIEEVGVLIEEPFPMLALDELCIVTQNNIKEAIATEKDIQAMLKGKRKRHSYKHSPNGCPNT

SEQ ID NO: 82 - protein, 416 aa, Gossypium raimondii
MAQSPNSPKLSLSSSFTSKPFLHFHSNSSLPAKFKRPPSFKILFSASQPPPPSSKPTTAPKTFNLITLLRAIPDWADRIQERGMQQNRALYNHEKWVHHRSSLRHLRHLLSSLQSRVILSLVPPVLAFTSVAIVVALYNTAVDLHWFPGFFPVLRASSLPYQLTAPALALLLVFRTEASYSRYEEGRKAWTKVIAGTNDFARQVIAGVDNSADDQSIKAALLQYIMAFPVALKCHVMYGSDIGRDLQNLLEVDDLALILNSKHRPRCIIEFISQSLQLLNLEESKRTMLESKISCFHEGIGVCEQLMGIPIPLSYTRLTSRFLVLWHLTLPIILWDDCHWIVVPATFISAASLFCIEEVGVLIEEPFPMLALDELCIVTQNNIKEAIATEKDIQAMLKGKRKRHSYKHSPNGCPNT

SEQ ID NO: 83 - protein, 417 aa, Nicotiana attenuata
MTNSRTLFSIQSPTNASFSSHFTLKLPSKLQQQSFPSKLSCKKFSFTTFKVRCCPQQTPQNQNSTSALISILRIIPDWADRIQEGGMKKKRSLYKHESWVQHRSSLRHVRHLFSSLNSRVILSLVPPVIAFTSVAVVIASYNSAVLMHWLPEFFPVLRASPLPYQLTAPALALLLVFRTEASYSRFETGKKAWTKVIAGTNDFARQVIACVDKSDAVLKEALLQYIMAFPVALKCHITYGSDIASDLKNLLEADDLALVLSSKHRPRCVIGFISQCLQSLNLEGTKLTQLESKISCFHEGIGVCEQLAGIPIPLSYTRLTSRFLVLWHLTLPIILWDDCHWIVVPATFISAASLFCIEEVGVLIEEPFPMLALDELCQLVRDNIQEAMANEKQIQERLNAKRKRRFSEHSQNGWPTS

SEQ ID NO: 84 - protein, 417 aa, Nicotiana sylvestris
MTNSRTLFSIQSPTNASFSSHFTLKLPSKLQQQSFPSKLSYKKFSFSTFKVRCCPQQTPENQNSTSALISILRIIPDWADRIQEEGMKKKRSLYKHESWVQHRSSLRHVRHLFSSLNSRVILSLVPPVIAFTSVAVVIASYNSAVSMHWLPEFFPVLRASPLPYQLTAPALALLLVFRTEASYSRFETGKKAWTKVIAGTNDFARQVIACVDKSDAVLKEALLQYIMAFPVALKCHITYGSDIASDLKNLLEADDLALVLSSKHRPRCVIGFISQCLQSLNLEGTKLTQLESKISCFHEGIGVCEQLAGIPIPLSYTRLTSRFLVLWHLTLPIILWDDCHWIVVPATFISAASLFCIEEVGVLIEEPFPMLALDELCQLVRDNIQEAMANEKQIQERLNAKRKKCFSEHSPNGWPTS

SEQ ID NO: 85 - protein, 417 aa, Nicotiana tabacum
MTNSRTLFSIQSPTNASFSSHFTLKLPSKLQQQSFPSKLSYKKFSFSTFKVRCCPQQTPENQNSTSALISILRIIPDWADRIQEEGMKKKRSLYKHESWVQHRSSLRHVRHLFSSLNSRVILSLVTPVIAFTSVAVVIASYNSAVSMHWLPEFFPVLRASPLPYQLTAPALALLLVFRTEASYSRFETGKKAWTKVIAGTNDFARQVIACVDKSDAVLKEALLQYIMAFPVALKCHITYGSDIASDLKNLLEADDLALVLSSKHRPRCVIGFISQCLQSLNLEGTKLTQLESKISCFHEGIGVCEQLAGIPIPLSYTRLTSRFLVLWHLTLPIILWDDCHWIVVPATFISAASLFCIEEVGVLIEEPFPMLALDELCQLVRDNIQEAMANEKQIQERLNAKRKKCFSEHSPNGWPTS

SEQ ID NO: 86 - protein, 417 aa, Nicotiana tomentosiformis
MTNSRTLFSIQSPTNASFSSHFTLKTPSKLQQQSFPSKLNFKKLRFSTFKVRCCPQQTPQNQNPTSALISILRIIPDWADRIQEEGMKKKRSLYTHESWMQHRSSLRHVRHLFSSLNSRVILSLVPPVIAFTSVAVVIASYNSAVSMHWLPELFPVLRASPLPYQLTAPALALLLVFRTEASYSRFETGKKAWTKVIAGTNDFARQVIACVDKSDAVLKAALLQYIMAFPVALKCHITYGSDIASDLKNLLEADDLAVVLSSKHRPRCIIGFISQCLQSLHLEGTKLTQLESKISCFHEGIGVCEQLAGIPIPLSYTRLTSRFLVLWHLTLPIILWDDCHWIVVPATFISAASLFCIEEVGVLIEEPFPMLALDELCQLVHDNIQESMANEKKIQERLSAKRKRRFSEHSQNGWPTS

SEQ ID NO: 87 - protein, 412 aa, Solanum pennellii
MTNPRTLFTIQSPTNASFSSHFTLKNPSKSQQQQPVYKKLNFRVLCSSQPSRTPQNSNLISTLVKILRVVPDWADKIQEGGMRKKRSLYKHETWVQHRSSLRHVRHLFSSFNSRVVLSLIPPVIAFTSFAFVIASYNSAVSFHWLPEFFPILRASPQPYQLTAPALALLLVFRTEASYSRFEAGKKAWTKVIAGTNDFARQVIACVDKRDDVLKEALLQYIMAFPVALKCHIVYDSDIASDLKNLLEADDLAVVLSSKHRPRCIIGFIAQSLQSLNLEGTILSQLESKISCFHEGIGVCEQLLGIPIPLSYTRLTSRFLVLWHLTLPIILWDDCHWIVVPATFISAASLFCIEEVGVLIEEPFPMLALDELCQRVHDNIHEAMANEKQIQEIVNSKRKRSFSEHSPNGWPTS

SEQ ID NO: 88 - protein, 412 aa, Solanum tuberosum
MTNPRTFFTIQSPTNASFSSHFTLKKPSNSQQQPPVYKKFTFRVLCSSQPSRTPQNSNLISTLVKILRVVPDWADKIQEGGMRKKRSLYKHETWVQHRSSLRHLRHLFSSFNSRVVLSLIPPVIAFTSFAFVIACYNSAVSLHWLPEFFPILRASPQPYQLTAPALALLLVFRTEASYSRFEAGKKAWTKVIAGTNDFARQVIACVDKRDDVLKEALLQYIMAFPVALKCHIVYDSDIASDLKNLLEADDLALVVSSKHRPRCIIGFISQSLQSLNLDGTILSQLESKITCFHEGIGVCEQLLGIPIPLSYTRLTSRFLVLWHLTLPIILWDDCHWIVVPATFISAASLFCIEEVGVLIEEPFPMLALDELCQRVHDNIHEAMANEKQIQEIVNSKRKRSFSEHSPNGWPTS

SEQ ID NO: 89 - protein, 481 aa, Chlorella sorokiniana
MVVAACRGHAMLSDAAQRRGPAAGQGPAAAAAIRGLSPCRRSASTWRLVPAAQRRPRPPMMDSILDDLGSNGDSAAESSTDLAAAEPPAVFPPLLSVDLASDPVKEGSRRYRRTVFDFNEWRKHRSTERYVRHMQSMTSSRMVRGLAAPLAYIFAVATAVSVYNASVEAGLLPSLLPEVKMASNGPFGSVSFAISLLLVFRTNASYARWLDARKAWGKLINRSRDLTRQALTYFPPEDKPLLDLLCRWTVAFSRTLKCHLCEDYPLAEELARVLRPHEAEALLLAKHRPTYCLQILSETLKSAHLPPPLPVAQPHNGATAGHAAAAQAVAAVPAAAAYRMDENLTEFEDIVGTCERILRAPIPLSYTRHTSRFMIIWLTLLPFALWDSVGWATVPLAVIVAFLLLGIEEIGVSIEEPFSILPLDALCDTIEANVKELQRTHSQQGGGGGEEGSHKARPPPARDIVGMAAAEAEAEEAAVAK

SEQ ID NO: 90 - protein, 590 aa, Chlorella sorokiniana
MSALLRARVSAFLAGVAVAGVFGVYQLRGDVAEGQQRLLDQTKKYTAGLEGRVAELEAAVARLSSNRALKPNSAAPPPAPAMPAGLMTQQSTACSAAAASGRLQGAARSLPRRHALSGAAVGRPAPAGLGRGLSSASRAQGRRAGAVRVQAAATPGVPGANSASEGPFDWMTDEWKEADRKNLRTVFDFDAWKRHRSSSRYWRHLGHLPNSRILKWVAGPLSYVTAVAIGVCAYHTAAEAGIVPEVLPELAKGAAAPFGLTSFALSTLLVLRTNTSYQRWDEARKMWGLIVNRSRDVARQALGYIPASQPELQDMFCRWLVAYCRSLMCHLRPGEDLRAELEGKLKPEELEALLSSAHRPNYTCQVLTAVIKRAQLPGDDIDNRDSFANVKASAAFRMDENLTQFADVTGGCERILRTPVPLMYTRHNSRFLMIWLSLLPFTLWDTCHWATVPVTALVAFLLLGIKEVGVAIEEPFTILPLEVICNTIEGNVWELHRMHSANGVEQQHAAGNAVVTAEALVDIMAPQTIGGLALNGAGSAAYVNGNGNGAYTNGAANGNGAESGTIKVPPEELIRAAQGLRQRVSSSLRQ

SEQ ID NO: 91 - protein, 450 aa, Chlorella sorokiniana
MATPAAAAPRRLGAAAAPKGLPRLAGVSARPRRAAQTAGSFHSAALPVDQAVNGNGTVPPDPEPEPTYSWFSQEWKEERRKKKGRTVFDFERWQKHRSSARYLRHMAGMFSSSVIQGLAVPLAYVVTVSVGVATYYTAAAKGMVPLFPALKVLASAPFSLTSFALSLLLVFRTNSSYGRWDEARKMWGQVVNRSRDIARQALAYIPAAQVHLQDVICRWTIAYTRALMCHLRQGEDLGSELAGILSPSELAALLAAKHRPNFCTQVLSAAVRDAQLPGTGPTSREPTAAVPAGAAYRMDENITVFVEVSGGCERILRTPIPLGYTRHTSRFLMLWLTLLPFSLWESCGLAMIPVAALVAFLLLGVEEIGVQIEEPFSILPLEVISSTIEGNIRELQATHGAAAAVPSPIHDIIRPPQLQSPEGNSSNVVGAPSLATNGAPAGRPSGGGAR

SEQ ID NO: 92 - protein, 408 aa, Chlorella sorokiniana
MQKRTRPRSRLPPVTLCCRLPPAAQPPRGVHAAQALLRPAGCSGSQQQRRRRRPPSTAASLLPLGDRDEDKEERLRRFRVMYDFDAWAAHRSVKRYWRHFIGLFRSRIVAGIALPILLVFLEATGVCWYETALRSGLLPATWRSIQVRAPMLFSLSSFALSLLLGFRTNQSIARFDEARRLWNTMINRCRDAMRQAVSYLGPQLPQQAALLGALSRWLQAFPWVLKNHVREHKHWQAELGDILEPSELEAVVASKHPPLLALQVITELLDRAPISAKQRLRIDGCVTDLSDVVGGCQRILNTPIPVSYTRHTCRYLTVWLFLLPLSIFNQCGWVTVFSSVVLAFFLLTIEEIGVTIEEPFNILPLAELNRGADVAQNLDRIDEVVALVDAATGSRRTAMQLAPAYPIE

SEQ ID NO: 93 - protein, 456 aa, Chlorella sorokiniana
MSTAMLAGSRIQLQQPAGLGGSRLQRAAAPVAAAARLGSVRPAGLQARSTAARRADRSALRVSATASPEAAPVKLSGDDLKEANRKHMRSVFDFDLWKKHRSSSRYLRHIVGLGESRIVSGLMAPLTYVMTLSLAVACYNAAAEAGYLPVFPELKLATNAPFGLTSFALSLLLVFRTNSSYGRWDEARKMWGLIVNRSRDFIRQGLGYIPPEQEELQKMLVRWTVAYSRSLMCHLRPGEDLRVELKDTLKPEELEALLASTHRPNYVVQVLTAIIKTAQLPAAVTNNRDSTGCVPAGAAYRMDENLTVFADVTGGCERILRTPIPLSYTRHTSRFMMIWLTLLPFTLWDSCHWAMLPIAGIVSFLLLGIEEIGVQIEEPFTILPLEVISRTIEGNVWELYRMHSGEALEKEQAELANGQDVQVLNAQDLVALMAPSAVGNTANGTSRKSLVVNYGL

SEQ ID NO: 94 - protein, 589 aa, Chlorella sorokiniana
MGLMPNMENTYSHVPRSAMAAMAASAPADASTNAGGSPGVGRLAARTSSEPPSGVHSRSSSLTNLARLPGKARAKLQNLTHSQFVRFIEDNDAQDKEENDIYRGAAAARQNPADQVFTFERWNKHRNPMRYAKHLLHMFTSRVFKQLLGPVLAVMALALSVGIYETLVSAGALPGHWPHVTLALGQGFNLTAFALSLLLVFRTNSSYDRWWEGRKLWGGVVNRTRNIVRQSLVFFREDVHLKELLARWTMAFPKVLMVHLREGMDLQAEVAHILTANEVAALVAAAHRPNFVLQVMAEAIRAARPHELARMRMDDNLTFFEDAMGSCERILRTPIPLSYTRHTSRFLLVWLILLPFTLWAAYSWFAVILSGIFAFLMFGIDEIGVQIEEPFGILPLEAAGATIERNIRELLARNYEVASLVHQQNLPVGLGDSRGKQPAGSSPAPAGGLGGTGGSAVSLIGLAAEGETEDFGEMTGASPADAAAVDAEGKLGGGARPSIIRLSATAGGAAPAASPRRVQFADDSTVIDVRSSDGGAVGARPVVISAAAPGGLDYRRQPLLLEVELASGASPLISPRDSPSSSYSSWLHD

SEQ ID NO: 95 - protein, 490 aa, Micractinium conductrix
MPAALCTGSAMQLQRRSPVTAAAPAAAAQRLVAPAAAAAPQHAASLRCSLRSTFPGGASISSVAALRTSAGRRSLHVAGAVGPNEKRAQIQQQRLDSLSKIDVLFDEELKALDTNLVKELVADDFKEQDRKKLRVVFDFDRWQRHRSSSRYLRHMTGLFTSRTLKWMGAPLVCTLSVATAVGIYYTAAEMGMVPDIPDIKTNAVFGLTSFALANLLVLTTNTAYQRFDEARKMWGLIVNRSRDMTRQGLAYIPEDQPELQEMFCRWVVAYSRSLMCHLRAGENLERELLGKLPESELRALLTATHRPNFTNQVIAQILKRADLPGAEVKPWDSTANVKASAFVRMDEHLTVFADVTGGCERILRTPVPLMYSRHTSRMLTIWLSFLPFSLWDACHWWTVPVTGLVAYLLLGIKEIGLTVEEPFSILPLEKICDTIESNVWELHSTHSAQGAAASRRRSAATRDATQSMDAEDLVSIAAAVDAGAMPAPAQ

SEQ ID NO: 96 - protein, 460 aa, Micractinium conductrix
MRLLPGCRRPGAAHGTAASLGCRPQRQRLRRAPLATAQPSDGDSSSSSNGTGLLSKVPAVLKSVDEDAVYVYDSGSDDVPLDILLLPLDRDDYKEDSRRHFRTVFDHNRWAAHRSTRRYVRHVLGMTSSRMVRGLAAPLFFVAGISAAVCAGHQAAEAGLLPLPEVAHQLQMRSTEPFALTSFALSLLLVFRTNSSYSRWLDARKNWGLVVTRSRDLVRQGITHIPHSERALIDLLCRWTAAFSRSLMAHLRADVDAEAELRRVLRPAEAEAALAAEHRPGYCLQVLSEIIKDAQLERPHAAATAAAATARPGQAAKPTRLDGGAACRMDENLTALEDMVGGCERILRTPIPLSYTRHTSRFMMIFLSLLPFALWESCRWATVPAVTIISFLLLGIEEIGVTIEEPFSILPLEQLCDVIEKNVWELHRCHVQPASAAIDGSGSRPLTAAGVVAAARLAED

SEQ ID NO: 97 - protein, 511 aa, Micractinium conductrix
MRTEPQTRLATGGAATQRRRSAPVVAGSAAGLPPPSPFASSRSGARTQRGAASPEQREVTWSLEDAGADMTINPDREAGRRLMRDLSNFSHRRWAFHRSTDRYWRHLAGIFQSRIGRQLFVPLSCAGGTAAAICAYETLLRDGALPAYFPSFVLPSLPFDFTAFALSLLLVFRTNTSYDRWQAAINAWGDISTRSRDTLRQLLAYTSATPAAAAVARDTSAAPLHGAGGSGAAAASAAQTTAAAGKWLVAFSRSLKAQLTEDSDLQAELKGVLTAGELELLTAAYHRPSFALAVLSELVAAAPVRDSQRARLDENLTTLEDMVGGCERLLRTPIPLSYTRHTSRFMVIWLSCLPLCLWNSCGWGTVPLTVVISFLLLGIEEIGVAIEEPFSIMPLHEMCAEMECGLADILEQAAPSKRAAAQAAAAAALTAASAAVGGSADAPADASGGGGGGGSTAGAAPAPALGVPAAAANSVAPFVLGMTAQRLRRPLSATPRPMDAIFCSMDDPDAR

SEQ ID NO: 98 - protein, 523 aa, Micractinium conductrix
MPRTENSYTHGARGSPSLDPEGSPLTQHARNSSLQNLARLPGVARAKLQNLSQTKFVRYIEDNDDGSRDENDIYRQHPGDQVFTFERWNKHRDSFRYAKHLAHMFSSRVFKQLLGPVLCVSALALAVGVYETLVAAGKLPGHWPHVTLALGQGFNLTAFALSLLLVFRTNSSYDRWWEARKLWGGVVNRTRDFVRQSLVFFRDEDAHLRELLAKWTIAFPHVLMCHLREHGDVAKAVAMMAEIVRTARPPELCRMRMDENLSFFEDAMGSMERILRTPIPLSLVWLILLPFTLWAAYSWFSVLLSGIFAFLMFGIDEIGVQIEEPFGILPLEAISSTIERNIRELLARNDEVVGLVAQAAQAGAVAAAAAAAADVAVAALPQGGQHDADAATVLAVPGDEFGEMVAAGSPVGKEPSAGNLITLTASSGEAAAAAAAAHHHLHAAARRVQFGGATVIEVGGSDEEAAGGGGGSPLGSPSHAHSPHRALNRQASMVEVELASGASPLLSPTDGSSAPVYSNWLRD

SEQ ID NO: 99 - protein, 425 aa, Micractinium conductrix
MTLLQQAVPSRVVVGGRQPRGIGSVLKPLRTSCRPAKAARGRQALPQASLQPAVAAGTAAPPPPSYVASANDERMEASRVEMRAVFDADRWIMHRSTDRYFRHLWGLPSSRVLQGLFKPIGYILAISAAVCAYGTAQAAGALPATLPLLASAIKLKELFGMTSFALSLLLVFRTNASYARWDEGRKMWGMVLNRTRNICRLGLAWIGDDKRELRSMLCRWAPAFSKTLMCHLRKGEDLREELEGILLPHEIEGVLRASHRPNYVLQVLAQIVRTAGLPTAATLRMEDDLTNFGDSLGGCERLLRTPIPLFYTRHTSRFLMIWLTFLPATLWPACGLLTLPLVFLISFLLLGVDKIGVSIEEPFSILALEVIAGTALANVRELVAMHEDTAAGVAAAANGNGGAAGAPAEADVSAAHMVTLAGAAA

SEQ ID NO: 100 - protein, 490 aa, Micractinium conductrix
MSALLRARVSSFLAGVAVAGVFGVYQLRGDVAEGQAQLLEQTKKYTSGLEARVAELEASVARLQKSSLLLASRPLGAGLVGLRSSQGGLERRRRASARVAAVASPVGDIEGRKPTVDERRESDLKELRSVFDFAAWKRHRSSDRYWRHMTGLWDSRTLDWVGPPLTYVMSVAALVCLYHAAADAGYVAEVIPELKPAATAPFSLTSFALSTLLVLRTNTSYQRWDEARKMWGLIVNRSRDISRQALGYIPATQPELQDMFIRWVVAYSRSLMCHLRAGEDLEKELKDTLGPEELKALMASAHRPNYTVQVLTSIIKAAQLPGEPISNYNSTANVKASAAFRMDENLTCFADVTGGCERILRTPVPLMYTRHNSRFLMIWLTLLPLTLWDSCHWFTMPVTGIVAFLLLGIKEIGVIVEEPFSILPLEKICDTIEGNVRELHATHSADAISAQLAVANAQNSGAASGGVDADTLVNTVVPAVTLRKPAVAAL

SEQ ID NO: 101 - protein, 1322 aa, Micractinium conductrix
MGASTDDSAAMADHPGRTPGGRPPPLSSSVGGSSRAKAMLAMATGGGGKAKTRTPSGRPTPPPITFERRKKRPQSEPGDGGGGEAAALGGETTPKRQRTPAGADCPSVPVAAPRPASLLAARPGSAAGSAGDAPRLTCYSNAQRLRELAAEGGDALARRRAALGGSGFIHRRPSSGGSLGRMQGLSRLPAGMMNLGNTCYLNAVLQVLLSLPSFVADLRHGRAALEAAGQPLAPSGVYSALLECAAARERGFERYLTPASLKRALDAASSAFHGTLQQDAHELFCSLLDAAQGEVLAREVARLGRTRVAVSETADPGARNFGFAMQHELVCGACGHTSQLVEQYTHLSLELPPAQPGGTAACSVASLLRDYFKEEQVEKACEACGGGGVAHTLRHAVKRLPRVLVLHLKRFKISWSAEQQRVVCQKVHTGVGMPETVQLGRLLAAGPAPPLLGGQQLGAGKENEQLEANAGVVGSAAQQQQQQQQRGDDSVAKGLRMREQAGFSFYSGGGGATPVAASALLSPGALHSPTLRRRLGTPAAPKLSNIVVDNDDAVLQRVLAESLREQQPGGGQLPGGTAQQQQQQQQQQQQQQQQQDGPELSLGPGGCLQRHSTEGAECPLIRCGSPGLRQAEAAPLLLPEVAAAAAAGSAPAGGCHKLEDQWFTGFRRGSPPAAPMAAEQEAAELQRVLDMSRREHERQAAAVAVRSSSAAGSAAQQEEADLLRAIELSMQEQRGKQPTSTQPQQHGGSQGTAGGDSSGGAGGAAAGAAAPGASAAAAAAAAAVTEQFDAGEAGKAEEAASAVQSVAPHGDDGVELVADAGGAAPAPLHAQIVEDNDAQLAAQRAQQAQHGSSGASADQQAAAEVTDAAEAAAGAEEKAAAAAAVRSGKPPAAEYRLHAVVNHKGPAASCGHFTSDIRDAASGVWHRFDDSLASRIAGWEARSSEKQRECYLLFYTGTVAGLIYSDRDEGKEFRLADFRTVYTFESWAAHRSTMRYLRHFMGMFRSRVLCGLRVPLLLIALEATLVCSYEHALKNGLLPAFCRTIQIRAPMLFSLSSFALSLLLVFRTNQSYARFDEGRRLWSVMANRCRDVMRQACSYLSLGPQNAALLATLSRWMQAFPWVMKNYVRESKSWHADLGSLLRPAELAALLDTRNPPLLTLQVITEILDRMPISTKQRLRLDIFMTELSDMVGASERLITTPIPLSYTRHTARFLITWLVLTPFTIFSSCGWVTIFSSVLLAFFLLTIEEIGVTIEEPFNILPLEAINTRVCADIARYPQKIDTVVSLVDDATGTQRPQQQPPFQPVAEFVSTAATSDNGDS

SEQ ID NO: 102 - protein, 1327 aa, Micractinium conductrix
MGASTDDSAAMADHPGRTPGGRPPPLSSSVGGSSRAKAMLAMATGGGGKAKTRTPSGRPTPPPITFERRKKRPQSEPGDGGGGEAAALGGETTPKRQRTPAGADCPSVPVAAPRPASLLAARPGSAAGSAGDAPRLTCYSNAQRLRELAAEGGDALARRRAALGGSGFIHRRPSSGGSLGRMQGLSRLPAGMMNLGNTCYLNAVLQVLLSLPSFVADLRHGRAALEAAGQPLAPSGVYSALLECAAARERGFERYLTPASLKRALDAASSAFHGTLQQDAHELFCSLLDAAQGEVLAREVARLGRTRVAVSETADPGARNFGFAMQHELVCGACGHTSQLVEQYTHLSLELPPAQPGGTAACSVASLLRDYFKEEQVEKACEACGGGGVAHTLRHAVKRLPRVLVLHLKRFKISWSAEQQRVVCQKVHTGVGMPETVQLGRLLAAGPAPPLLGGQQLGAGKENEQLEANAGVVGSAAQQQQQQQQRGDDSVAKGLRMREQAGFSFYSGGGGATPVAASALLSPGALHSPTLRRRLGTPAAPKLSNIVVDNDDAVLQRVLAESLREQQPGGGQLPGGTAQQQQQQQQQQQQQQQQQDGPELSLGPGGCLQRHSTEGAECPLIRCGSPGLRQAEAAPLLLPEVAAAAAAGSAPAGGCHKLEDQWFTGFRRGSPPAAPMAAEQEAAELQRVLDMSRREHERQAAAVAVRSSSAAGSAAQQEEADLLRAIELSMQEQRGKQPTSTQPQQHGGSQGTAGGDSSGGAGGAAAGAAAPGASAAAAAAAAAVTEQFDAGEAGKAEEAASAVQSVAPHGDDGVELVADAGGAAPAPLHAQIVEDNDAQLAAQRAQQAQHGSSGASADQQAAAEVTDAAEAAAGAEEKAAAAAAVRSGKPPAAEYRLHAVVNHKGPAASCGHFTSDIRDAASGVWHRFDDSLASRIAGWEARSSEKQRECYLLFYTGTVAGLIYSDRDEGKEFRLADFRTVYTFESWAAHRSTMRYLRHFMGMFRSRVLCGLRVPLLLIALEATLVCSYEHALKNGLLPAFCRTIQIRAPMLFSLSSFALSLLLVFRTNQSYARFDEGRRLWSVMANRCRDVMRQACSYLSLGPQNAALLATLSRWMQAFPWVMKNYVRESKSWHADLGSLLRPAELAALLDTRNPPLLTLQVITEILDRMPISTKQRLRLDIFMTELSDMVGASERLITTPIPLSYTRHTARFLITWLVLTPFTIFSSCGWVTIFSSVLLAFFLLTIEEIGVTIEEPFNILPLEAINTRAINTRVCADIARYPQKIDTVVSLVDDATGTQRPQQQPPFQPVAEFVSTAATSDNGDS

SEQ ID NO: 103 - protein, 333 aa, Physcomitrella patens
MHRNSVYGHGEWKKHKSSWRHVRHVCTILSSGLIRAIGPPVFLCTLVSVFVAVINHCVDNGVLPSWFPVLKVATLPFTLTSPVLALLLVFRTNTSYQRFDEARKAWGSNVNRARDLARQALTWIRNPGDSKKLQCLLRYTKAYSFCLMHHLREEGCLRKELEATIVNEEEVECVMNSKNRPIWVLQVISDIINECQITPWERIAMDKNITQFHDNVGACERIFKTPIPVAYTRLTSRVLTLWHLVLPFALWETCGWHTITVSFVSSAALFYIEEVGVMIEEPFSILALSTICTGIVAALEGLSNAHTDALILVWGIVEQQWSTKADHLMINLK

SEQ ID NO: 104 - protein, 402 aa, Physcomitrella patens
MHRNTVYGHDEWRRHKSSWRHARHVISIAASGVIAALGPPVLLSTITAVFVTVINHGVQHMLVASWVPYLKVSPIPFTFISPVLAFLLVFRTNSSYQRFDEARKVWGSNVNRCRDLARQALSWIKNPEDAARLECLLRFLKAYPYYLKLHLTQEGPSSSTTSEIKDILKDEEFHKVSLVQNQPIYVLQVISEIISQCHIPNWEKICMDANLTQFHDNVGACERIFKTPIPIAYTRMTSRMLIMWHLALPYGLWNDCRWLTIPATFMSSAALFYIEQVGVVIEEPFCILALDSICGGIRSAIDMFRATREDVFLLTESIRSEHPKKTDTTKNLEQIIEMESMDSDPLEETSKEDEVMLGLGRSKSYKSYSGRKFFDKSRSHSHSTTSLYGDLNVGFSKLKSVS

SEQ ID NO: 105 - protein, 353 aa, Physcomitrella patens
MHRNTVYGHTEWRRHKSCWRHSRHLSSIFSSGVIFTLGPPVILCTLVATLVSVINHLVQDRRLPKWMPLLHVASLPFTLTAPVLALLLVFRTNASYSRFDEARKAWGSNVNRTRDLARQALTWIRMPCDAPKLHCLLRHIKAYSLCLKDHMTEDNTLREELTAVLEPSEVDCAMSSQHRPNYILQVMSELISQCQISQWEKITMDKNITAFHDNVGACERIFKTPIPLAYTRLTSRMLMFWHLALPVGLWNTCGWLTIPVSFMSAAALFYIEEVGVLIEEPFWILPLLSISQGICSAVDGLYVAHKEASMLWSIHQPTNVNDHVIISFDGSRSAGIYQHNNIDVKFSRVNSVG

SEQ ID NO: 106 - protein, 353 aa, Physcomitrella patens
MHRNKVYGHAEWAKHKSSWRHGRHLMSILSSGVISAVGPPVLACTLLATFVTVFNYFVKVGRLPNWIPILEVSSLPFTLTSSVLSLLLVFRTNSSYNRFDEARKIWGSNVNRTRDLARQALSWIRSPADAYKLSCLLRHIKAYPFSLKDHLTEDFILKDELDQILEPQELEALMATKHRPNYILQVLSELVDKCNLSEWEKMAMDENITTFHDNVGACERILKTPIPLAYTLVTSRFLILWHLVLPFALWATCHWLTIPVTFLTATALFYIEEVGVLIEEPFWILPLLSISDGIVAALDGLAAAHKTSSLLWRLHQPNKDHVVQITFETSRSDGFRKSRDVGHVLLTRMNSIA

SEQ ID NO: 107 - protein, 391 aa, Physcomitrella patens
MFWKHPSGRTASSEGFVIKPPDSVQQEQQSVDTAGFSLLFHPNALSHKHKVSDEKVAWGETSKGGVSGGGGGGSVTLEFFSFKNASQRSVWARRMGLHKSSWRHGRHIQTAFSTGVISSIIPRVFFCTLISVLVTIFNHAVMEGVLPHWVPSLRVPTLPMSLTAPVLSLLLVFRTNSSYNRLDEARKAWGSNVNRTRDVSRQALSWICDPDDADKLQSLLRHIKAFSYCLKDHLTQENLLQEELARVLEPREVELVLKSSHRPNYVLHVMSDTIKHCRISKWESKSMDRNITQFHDNVGACERLFKTPIPVAYTRMISRFLSIWHFLLPLALWNSCRWLTIPVTFVSGVGLFCIEEVGVLIEDPSTSLLCCQSAMAFPQLWMASARLMKEL

SEQ ID NO: 108 - protein, 518 aa, Auxenochlorella protothecoides
MPGALATSLDEPDRPQRSVSKTLKRAVSSYFVPRGRFKKLLESAENDNEADVYRRNSGHRVFTHADWAVHRKPSRYARHLALIFASRIFRSLIPPITAVTLISTLVGVYETLLKEHRLPAQWPHVTLALGQGFQLTAFALSLLLVFRTNSAYGRWWEARMLWGSLTNRTRDFVRQCLAFFPEDAKRLREAAVLWTIALPYVVKAHLREGNRLESELEGILTRDEVDALTGATHRPCFVLAALSQVVAAAGLDPQLRQRLDTNLTAFEDCIGGCERILRTPIPQSYTRHTSRFLMIWLIMMPFTLWAAYSWVSILLSGLFAYLMLGIDEIGVQIEEPFGVLPLGSFCATIERNLRELHYSNPEARFAALPWLVPASPTCSRASGSCDGADMAAGMDDIDPVAGKLPHLNGAPQAGQPRVYKHTSDAAELAAGHVRFAHRWLEPHPHLIHSPVHPHHPNHHHGHGHGHGAQEHGRGAFHQTSTLHADVEDQVVHPASYPTAARHTPPHRSWFGGRWVEED

SEQ ID NO: 109 - protein, 412 aa, Capsicum baccatum
MTNPRSLFNIQSPTNASLSSHFTLKKPSKLLSYKKLTFCSFKVLCSSQPSKAPQNSNLTSTLVSILRVIPDWADKIQEGGMRKKRSLYKHETWVQHRSSLRHLRHLFSSFNSRVVLSLIPPVIAFTSFAFVIASYNSAVSLHWLPEFFPLLRASPQPYQLTAPALALLLVFRTEASYARFETGKKAWTNVIAGTNDFARQVITACVDKRDGVLKEALLQYIMAFPVALKCHIVYGSDLASDLKNLLEADDLAVVLSSKHRPRCIIGFISQSLQSLNLEGTKLSQLESKISCFHEGIGVCEQLIGIPIPLSYTRLTSRFLVLWHLTLPIILWDDCHWLVVPATFISAASLFCIEEVGVLIEEPFPMLALDELCQRVHDNIQEAMASEKQIQERLNTKRKRHFSENSPNGWPTS

SEQ ID NO: 110 - protein, 435 aa, Coccomyxa subellipsoidea
  source 1..435: note = C-169
MIATIHTLYYQAGLQKGFQTPRPRWRKLHYSRAPGKLSRNLLVRCQETDIEWVERAAIQREAWKEESRKYRRVTFTYDDWVAHRSTTRYSRHLAGILDSRIFRGLLPTLTAVMMVATFVSVYETLREAGILIPHDWVGLTVEAGQAFNLTSFALSLLLVFRTNESYSRWLEARKAWTNIVTRSRDFARQGLSWLSGDKQRESMLERWTIAFIKCTMAHVREECDLQSLLEDVLEAHELEQLMAAQHKPNFVLHVLSELVWGANLMEGQAMRMDEALTVFGEQVGTCERLLKTPIPLSYTRHTSRFLVMWLAFLPFSLWDACHWVTIPASGIIAFLLLGIEEIGVQIEEPFGILPLGAWILFHTIEADSVIDPQSFDCTIQQSVKGALSLALLLRMTGCARRAIQRCCCGRDQTDGARHECASSGGGRPERERCKR

SEQ ID NO: 111 - protein, 474 aa, Coccomyxa subellipsoidea
  source 1..474: note = C-169
MSRLNHHALFEDHVVTAADWYHLRSWRRHLPDLTWVTVLSGFWLWVVYFALIAVLIGLYHTVLVPRGAPDWPPKNVASVLYQPFAITSFALALLMVFRTNSSYARWWEARTVWGQVFNVTRNLVRQADAWFGEEDVAAFQMLVRWCAAAGYILQAHLCTAKLGPEAEGLLHPEELKLLASWEHRPICAGQVLSSIVASAVKDSQLRAAMDDQIATYINDAGACERIQRTCIPFCYTRHTSRFLILWLTFLPFALWEICGWASPVAEAVLAFLLMGVENIGIQIEEPFHVLPMHSYCAVIAKNALEVARERKGLDLFVEEALRRELCMEPLGKLENGGAGADSNVFVRPSGAAGVSHKASPRPTNPSLRVRCQSHRHNADPSLHEARGVFQMVPQRGEATLLDARDALETCSALNDASSKEACYATFGCDGRRVEKYFGAVEVLETAWEQVKEEEVEDDDEFVEENLSPWPWDGR

SEQ ID NO: 112 - DNA, 20 bp, synthetic construct
  misc_feature 1..20: note = Synthetic Construct
  source 1..20: mol_type = other DNA
gacaccaagaccatcctggc

SEQ ID NO: 113 - DNA, 21 bp, synthetic construct
  misc_feature 1..21: note = Synthetic Construct
  source 1..21: mol_type = other DNA
aacagaactgcagaggtcccg

SEQ ID NO: 114 - DNA, 17 bp, synthetic construct
  misc_feature 1..17: note = Synthetic Construct
  source 1..17: mol_type = other DNA
cggtgcccatgagctcc

SEQ ID NO: 115 - DNA, 17 bp, synthetic construct
  misc_feature 1..17: note = Synthetic Construct
  source 1..17: mol_type = other DNA
gccactaaccggcccaa

SEQ ID NO: 116 - DNA, 18 bp, synthetic construct
  misc_feature 1..18: note = Synthetic Construct
  source 1..18: mol_type = other DNA
aatcccgtccatgtcgct

SEQ ID NO: 117 - DNA, 18 bp, synthetic construct
  misc_feature 1..18: note = Synthetic Construct
  source 1..18: mol_type = other DNA
cggcttgtgaggacctcg

SEQ ID NO: 118 - DNA, 20 bp, synthetic construct
  misc_feature 1..20: note = Synthetic Construct
  source 1..20: mol_type = other DNA
gccagaaggactcgtacgtt

SEQ ID NO: 119 - DNA, 20 bp, synthetic construct
  misc_feature 1..20: note = Synthetic Construct
  source 1..20: mol_type = other DNA
cgccagagtccagcacgata

SEQ ID NO: 120 - DNA, 48 bp, synthetic construct
  misc_feature 1..48: note = Synthetic Construct
  source 1..48: mol_type = other DNA
gctactcacaacaagcccagttatgcagatgcaagcaaaccgttcgtc

SEQ ID NO: 121 - DNA, 40 bp, synthetic construct
  misc_feature 1..40: note = Synthetic Construct
  source 1..40: mol_type = other DNA
gagccacccagatctccgttcttgcgctccccacccatgg

SEQ ID NO: 122 - DNA, 40 bp, synthetic construct
  misc_feature 1..40: note = Synthetic Construct
  source 1..40: mol_type = other DNA
gctactcacaacaagcccagttatggccactggtcagacc

SEQ ID NO: 123 - DNA, 37 bp, synthetic construct
  misc_feature 1..37: note = Synthetic Construct
  source 1..37: mol_type = other DNA
gagccacccagatctccgtttctccttgtctccgcac

SEQ ID NO: 124 - DNA, 46 bp, synthetic construct
  misc_feature 1..46: note = Synthetic Construct
  source 1..46: mol_type = other DNA
gctactcacaacaagcccagttatgcaagtcagcaaggttccctcg

SEQ ID NO: 125 - DNA, 38 bp, synthetic construct
  misc_feature 1..38: note = Synthetic Construct
  source 1..38: mol_type = other DNA
gagccacccagatctccgttccggggcgagatgcgcac

SEQ ID NO: 126 - DNA, 90 bp, synthetic construct
  misc_feature 1..90: note = Synthetic Construct
  source 1..90: mol_type = other DNA
ctagtgggagcgagttgcaaggcatatctcgctgatcggcaccatgggggtggtggtgatcagcgctatatgttttgcaactcgctcccg

SEQ ID NO: 127 - DNA, 90 bp, synthetic construct
  misc_feature 1..90: note = Synthetic Construct
  source 1..90: mol_type = other DNA
ctagcgggagcgagttgcaaaacatatagcgctgatcaccaccacccccatggtgccgatcagcgagatatgccttgcaactcgctccca

SEQ ID NO: 128 - DNA, 90 bp, synthetic construct
  misc_feature 1..90: note = Synthetic Construct
  source 1..90: mol_type = other DNA
ctagtgagagcgtgttgcaaggcatatctcgctgatcggcaccatgggggtggtggtgatcagcgctatatgttttgcaacacgctctcg

SEQ ID NO: 129 - DNA, 90 bp, synthetic construct
  misc_feature 1..90: note = Synthetic Construct
  source 1..90: mol_type = other DNA
ctagcgagagcgtgttgcaaaacatatagcgctgatcaccaccacccccatggtgccgatcagcgagatatgccttgcaacacgctctca

SEQ ID NO: 130 - DNA, 20 bp, synthetic construct
  misc_feature 1..20: note = Synthetic Construct
  source 1..20: mol_type = other DNA
gctgtgtggcattgaggaga

SEQ ID NO: 131 - DNA, 20 bp, synthetic construct
  misc_feature 1..20: note = Synthetic Construct
  source 1..20: mol_type = other DNA
ggatgaggctgatgagtccg

SEQ ID NO: 132 - DNA, 20 bp, synthetic construct
  misc_feature 1..20: note = Synthetic Construct
  source 1..20: mol_type = other DNA
acggtctacgacttccctca

SEQ ID NO: 133 - DNA, 20 bp, synthetic construct
  misc_feature 1..20: note = Synthetic Construct
  source 1..20: mol_type = other DNA
ttggatcacgtgggattggg

SEQ ID NO: 134 - DNA, 20 bp, synthetic construct
  misc_feature 1..20: note = Synthetic Construct
  source 1..20: mol_type = other DNA
aagtcagcaaggttccctcg

SEQ ID NO: 135 - DNA, 20 bp, synthetic construct
  misc_feature 1..20: note = Synthetic Construct
  source 1..20: mol_type = other DNA
tgaatgagcctagcgggttg

SEQ ID NO: 136 - DNA, 20 bp, synthetic construct
  misc_feature 1..20: note = Synthetic Construct
  source 1..20: mol_type = other DNA
atgtgctgtccgtggctttc

SEQ ID NO: 137 - DNA, 22 bp, synthetic construct
  misc_feature 1..22: note = Synthetic Construct
  source 1..22: mol_type = other DNA
cagaccttgaccatcttgtccc

SEQ ID NO: 138 - DNA, 18 bp, synthetic construct
  misc_feature 1..18: note = Synthetic Construct
  source 1..18: mol_type = other DNA
tgccccttctcagcacgt

SEQ ID NO: 139 - DNA, 18 bp, synthetic construct
  misc_feature 1..18: note = Synthetic Construct
  source 1..18: mol_type = other DNA
actgcctcacactcccct

SEQ ID NO: 140 - DNA, 20 bp, synthetic construct
  misc_feature 1..20: note = Synthetic Construct
  source 1..20: mol_type = other DNA
gcaccaatcatgtcaagcct

SEQ ID NO: 141 - DNA, 20 bp, synthetic construct
  misc_feature 1..20: note = Synthetic Construct
  source 1..20: mol_type = other DNA
gacgttacagcacacccttg

(19) United States
(12) Patent Application Publication
(10) Pub. No.: US 2026/0078396 A1
(43) Pub. Date: Mar. 19, 2026

(54) GREEN ALGAL BESTROPHIN BICARBONATE TRANSPORTERS

(71) Applicants: Board of Supervisors of Louisiana State University and Agricultural and Mechanical College, Baton Rouge, LA (US); University of York, York (GB)

(72) Inventors: Ananya MUKHERJEE, Baton Rouge, LA (US); James V. MORONEY, Baton Rouge, LA (US); Chun Sing LAU, York (GB); Luke C.M. MACKINDER, York (GB)

(73) Assignees: Board of Supervisors of Louisiana State University and Agricultural and Mechanical College, Baton Rouge, LA (US); University of York, York (GB)

(21) Appl. No.: 19/251,381

(22) Filed: Jun. 26, 2025

Related U.S. Application Data
(63) Continuation of application No. 17/259,537, now Pat. No. 12,378,568.
(60) Provisional application No. 62/697,840, filed on Jul. 13, 2018.
(60) Provisional application No. 62/769,214, filed on Nov. 19, 2018.

(51) Int. Cl. C12N 15/82

(52) U.S. Cl. CPC C12N 15/8261; C12N 15/8269

(57) ABSTRACT

Aspects of the present disclosure relate to genetically modified plants and/or algae with increased carbon use efficiency as a result of an increased ability for bicarbonate to cross membranes within plant cells. Other aspects of the present disclosure relate to methods of making such plants and/or algae as well as cultivating these genetically modified plants to increase carbon use efficiency and/or growing these genetically modified algae to increase carbon use efficiency.

### CROSS-REFERENCE TO RELATED APPLICATIONS

This application is a continuation of U.S. application Ser. No. 17/259,537, filed Jan. 11, 2021, which is a national stage application under 35 U.S.C. § 371 of International Application No. PCT/US2019/041584, filed Jul. 12, 2019, which claims the benefit of U.S. Provisional Application No. 62/769,214, filed Nov. 19, 2018, and U.S. Provisional Application No. 62/697,840, filed Jul. 13, 2018, which are hereby incorporated by reference in their entirety.

### REFERENCE TO AN ELECTRONIC SEQUENCE LISTING

The contents of the electronic sequence listing (794542000101SEQLIST.xml; Size: 193,915 bytes; and Date of Creation: Jun. 24, 2025) is herein incorporated by reference in its entirety.

### TECHNICAL FIELD

The present disclosure relates to genetically modified plants. In particular, the present disclosure relates to genetically modified plants containing green algal bestrophin bicarbonate transporters that preferably provide increased carbon use efficiency.

### BACKGROUND

Green algae and other photosynthetic aquatic organisms are often exposed to low and fluctuating CO₂ conditions in the natural environment. There are a variety of factors that can reduce CO₂ availability for these organisms, including slow diffusion of gases in water, slow interconversion of the two inorganic carbon (Ci) forms carbon dioxide (CO₂) and bicarbonate (HCO₃), and pH changes. As a result of the changeable natural environment, most aquatic photosynthetic organisms have evolved a carbon dioxide concentrating mechanism (CCM) that is inducible under limiting CO₂ conditions. The CCM allows aquatic photosynthetic organisms such as green algae to effectively concentrate Ci for fixation by Rubisco (Giordano et al., Ann Rev Plant Bio 56: 99-131, 2005). The current CCM model for the green alga Chlamydomonas reinhardtii (Jungnick et al., Photosynth Res 121: 159-173, 2014; Wang and Spalding, Plant Physiol 166:2040-2050, 2014) includes bicarbonate transporters on the plasma membrane and chloroplast envelope as key components of the CCM allowing the movement of Ci, particularly HCO₃⁻, through the membranes. Carbonic anhydrase enzymes that interconvert CO₂ and HCO₃⁻ are a further important component of the CCM model (Mitra et al., Can J Bot 83: 780-795, 2005; Moroney et al., Photosynth Res 109: 133-149, 2011).

In C. reinhardtii, a compartment called the pyrenoid is located at the base of the chloroplast. The pyrenoid is the location where Rubisco is sequestered under limiting CO₂ conditions (Kuchitsu et al., Plant Cell Phys 29: 1269-1278, 1988; Rawat et al., Planta 198:263-270, 1996; Borkhsenious et al., Plant Physiol 116: 1585-1591, 1998). An extensive network of thylakoid tubules and mini-tubules is associated with the pyrenoid (Engel et al., Elife 13:04889, 2015), presumably to provide a pathway for HCO₃⁻ to enter into the pyrenoid. The thylakoid carbonic anhydrase CAH3 is found in these tubules and it is hypothesized that CAH3 converts HCO₃⁻ to CO₂ for fixation within the lumen (Moroney and Ynalvez, Eukaryotic Cell 6: 1251-1259, 2007).

C. reinhardtii cells grown under high CO₂ conditions (5% v/v) exhibit a low affinity for Ci. When high CO₂ acclimated cells are exposed to lower CO₂ conditions (e.g., ambient 0.04% to low <0.01% v/v), induction of high affinity Ci transporters has been reported. While CO₂ will readily diffuse across membranes in the cell (Gutknecht et al., J. Gen. Physiol 69: 779-794, 1977), numerous studies have since established the need for an active transport system to facilitate the movement of Ci (particularly HCO₃⁻) to the location where fixation by Rubisco can occur in cells grown under low CO₂ conditions (Moroney et al., Plant Physiol 83: 460-463, 1987; Sultemeyer et al., Planta 176: 256-260, 1988; Badger et al., Physiologia Plant 90: 529-536, 1994; Ohnishi et al., Plant Cell 22: 3105-3117, 2010). In addition, molecular and physiological studies have also confirmed the occurrence of multiple forms of Ci transporters on the plasma membrane and chloroplast envelope of cells (Amoroso et al., Plant Physiol 116:193-201, 1998; Duanmu et al., PNAS 106: 5990-5995, 2009; Atkinson et al., Plant Biotechnol J 5: 12497, 2015; Gao et al., Plant 82: 1-11, 2015; Yamano et al., PNAS 112: 7315-7320, 2015).

In cyanobacteria that live in marine environments, HCO₃⁻ transport at the plasma membrane is often coupled to the high external Na⁺ ion concentration of saltwater. In the freshwater environments where C. reinhardtii is found, transport is thought to be H⁺-coupled since Na⁺ concentration is relatively low (Morth et al., Nat Rev Mol Cell Biol 12: 60-70, 2011; Taylor et al., Trends Plant Sci 17: 675-684, 2012). Consequently, genomic studies with C. reinhardtii and Volvox carteri have revealed the presence of both H⁺- and Na⁺-coupled transporters for sulphate and phosphate (Pootakham et al., Plant Physiol 153: 1653-1668, 2010). It is not yet clear whether this type of ion coupling is also important for bicarbonate uptake.

To date, two high affinity bicarbonate transport proteins and one low affinity bicarbonate transport protein in C. reinhardtii have been characterized and are known to be functional under low CO₂ conditions. The first high affinity transporter, High Light Activated protein 3 (HLA3), is an ATP-binding cassette (ABC)-type transporter of the Multi-Drug Resistance protein family, and is localized to the plasma membrane (Im and Grossman, 2002). The Hla3 transcript is induced by both high light and low CO₂ conditions and is controlled by the CCM ‘master regulator’ encoded by the Cia5 gene. Duanmu et al. (2009) showed a significant reduction in Ci affinity and Ci uptake in HLA3 RNAi knockdown mutants, supporting the role of this protein in HCO₃⁻ transport. The second high affinity transporter, Low Carbon Inducible protein 1 (LCI1), is a relatively small protein. LCI1 is strongly upregulated in cells grown under low CO₂ conditions, and it has been localized to the plasma membrane (Ohnishi et al., Plant Cell 22: 3105-3117, 2010). In addition, overexpression of the LCI1 protein in the Lcr1 (Chlamydomonas strain lacking a MYB-transcription factor) background resulted in increased Ci uptake. Thus, HLA3 and LCI1 are both thought to be Ci transporters located on the plasma membrane.

The third transporter, NAR1.2 (also known as LCIA), is a chloroplast envelope protein of the Formate/Nitrite transporter family. Although the NAR1.2 protein has lower affinity for bicarbonate (as revealed in the K_{1/2} value which falls in the mM range), expressing NAR1.2 in Xenopus laevis oocytes resulted in increased HCO₃⁻ uptake (Mariscal et al., Protist 157: 421-433, 2006; Atkinson et al., Plant Biotechnol J 5: 12497, 2015). NAR1.2 has been shown to be localized to the chloroplast envelope, and is believed to be involved in Ci uptake, but the molecular mechanism for this remains unclear (Yamano et al., PNAS 112: 7315-7320, 2015). Experimental results indicate that NAR1.2 and HLA3 proteins have a cooperative role within the CCM (Yamano et al., PNAS 112: 7315-7320, 2015). Under very low CO₂ conditions, NAR1.2 has been shown to act in conjunction with Low-CO₂ Inducible protein B (LCIB). These results suggested a model in which LCIB is involved in CO₂ uptake and recapture of CO₂ that leaks from the pyrenoid, while NAR1.2 is involved in the HCO₃⁻ uptake and transport pathway (Wang and Spalding, Plant Physiol 166:2040-2050, 2014).

In addition to the proteins likely involved in the CCM that are described above, roles for multiple other proteins have been suggested. For example, the two soluble proteins LCIB and LCIC form a complex that has been observed to closely associate with the pyrenoid when cells are acclimated to very low CO₂ (Yamano et al., Plant Cell Physiol 51: 1453-1468, 2010), but it is not yet certain what the role of this complex might be (Jin et al., PNAS 113:14716-14721, 2016). Another example is provided by CCP1 and CCP2, which are other putative Ci transporters that have been shown to be localized to mitochondria (Atkinson et al., Plant Biotechnol J 5: 12497, 2015). This localization suggests that mitochondria may be important in CCM function, but it is not clear yet what the mechanisms of this might be or what role mitochondria could play.

Bestrophins are a family of membrane proteins that exhibit Cl⁻ channel activity and also function as regulators of voltage-gated Ca²⁺ channels. Human and mouse bestrophins have been found to have high permeability and conductance to HCO₃⁻ (Qu and Hartzell, Am J Physiol Cell Physiol 294: C1371-C1377, 2008). However, it has been shown that amino acids in phototrophic bestrophin proteins show high diversity compared with their counterparts in mammals using protein alignment and phylogenetic tree analyses. For example, the residues forming the Ca²⁺ sensing apparatus in animal bestrophin are not conserved in the bestrophin-like proteins in Arabidopsis (AtBest1 and AtBest2), suggesting that Ca²⁺ is not required for AtBest channel activation in the chloroplast. Therefore during evolution, bestrophin proteins in phototrophs may have acquired different electrophysiological properties from those in mammals (Duan et al. Journal of Integrative Plant Biology 58: 848-858, 2016). In C. reinhardtii, LCI11 and Cre16.g662600 have been suggested as putative bestrophins, and Cre16.g663400 is proposed to be a bestrophin-like protein (Mackinder et al., Cell 171:133-147, 2017), but these have not been further characterized.

Current data only unambiguously support the necessity of five proteins for the inorganic carbon (Ci) influx in C. reinhardtii, including HLA3, LCI1, LCIA, CAH3, and LCIB (Mackinder, New Phytologist 217: 54-61, 2018). As described above, these proteins are localized to the plasma membrane and the chloroplast envelope. The current model of the C. reinhardtii CCM suggests that at least one additional transporter or channel in the chloroplast thylakoid membrane is required to maintain a flux of HCO₃⁻ from the stroma to CAH3 in the lumen (Mackinder New Phytologist 217:54-61, 2018). Moreover, identifying functional bicarbonate transport proteins remains an important goal. Current research has identified many possible bicarbonate transport proteins, but only a few of these have promising experimental results, and there is little mechanistic data available.

### BRIEF SUMMARY

In order to meet these needs, the present disclosure is directed to green algal bestrophin polypeptides that act as bicarbonate transport proteins. Certain aspects of the present disclosure relate to a genetically altered plant or part thereof containing one or more genetic alterations that increase or provide the ability for bicarbonate to cross a membrane from a plant cell cytoplasm into a stroma of at least a portion of the chloroplasts of the plant. In some aspects, the present disclosure relates to a genetically altered plant or part thereof containing one or more genetic alterations that increase or provide the ability for bicarbonate to cross a membrane from a stroma into a lumen of at least a portion of the chloroplasts of the plant. In some embodiments, the gain of the bicarbonate membrane crossing ability is the result of the expression of at least one green algal bestrophin polypeptide. In some embodiments, the green algal bestrophin polypeptide is selected from the group of SEQ ID NO:1, SEQ ID NO:2, or SEQ ID NO:3. In some embodiments, the increase or provision of the ability for bicarbonate to cross a membrane is the result of expression of a polypeptide selected from the group of a first polypeptide with at least 70% sequence identity, at least 75% sequence identity, at least 80% sequence identity, at least 85% sequence identity, at least 90% sequence identity, or at least 95% sequence identity to SEQ ID NO:1, a second polypeptide with at least 70% sequence identity, at least 75% sequence identity, at least 80% sequence identity, at least 85% sequence identity, at least 90% sequence identity, or at least 95% sequence identity to SEQ ID NO:2, a third polypeptide with at least 70% sequence identity, at least 75% sequence identity, at least 80% sequence identity, at least 85% sequence identity, at least 90% sequence identity, or at least 95% sequence identity to SEQ ID NO:3, or any combination thereof. In some embodiments, the increase or provision of the ability for bicarbonate to cross a membrane is the result of expression of a polypeptide selected from the group of a first polypeptide with at least 95% sequence identity to SEQ ID NO:1, a second polypeptide with at least 95% sequence identity to SEQ ID NO:2, a third polypeptide with at least 95% sequence identity to SEQ ID NO:3, or any combination thereof. In some embodiments, the increase or provision of the ability for bicarbonate to cross a membrane is the result of expression of a polypeptide selected from the group of SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, or SEQ ID NO:111, preferably selected from the group of SEQ ID NO:10, SEQ ID NO:11, SEQ ID NO:15, SEQ ID NO:27, SEQ ID NO:28, SEQ ID NO:29, SEQ ID NO:42, SEQ ID NO:62, or SEQ ID NO:63. In some embodiments, the polypeptide is localized to a chloroplast envelope or a thylakoid membrane of at least one chloroplast within a plant cell. In some embodiments, the plant cell is a leaf mesophyll cell. In some embodiments, the polypeptide is expressed in at least 70% of leaf mesophyll cells of the plant.

In some aspects, the present disclosure relates to plants or parts thereof with increased carbon use efficiency containing at least one modified nucleic acid sequence with at least one coding sequence of a green algal bestrophin polypeptide in the plant or part thereof, where the bestrophin polypeptide is expressed in the plant or part thereof, and where when the plant is cultivated under ambient carbon dioxide conditions, the yield, growth rate, or biomass is greater than from a corresponding wild-type (WT) plant or corresponding WT part thereof that does not overexpress the bestrophin polypeptide or the yield, growth rate, or biomass is substantially similar to the yield, growth rate, or biomass from the corresponding wild-type (WT) plant or corresponding WT part thereof that does not overexpress the bestrophin polypeptide cultivated under ambient carbon dioxide conditions. In some embodiments, the bestrophin polypeptide is localized to a chloroplast envelope or a chloroplast thylakoid membrane of at least one chloroplast of a plant cell. In some embodiments, the plant cell is a leaf mesophyll cell. In some embodiments, the polypeptide is expressed in at least 70% of leaf mesophyll cells of the plant.

In some aspects, the present disclosure relates to plants or parts thereof with increased water use efficiency containing at least one modified nucleic acid sequence with at least one coding sequence of a green algal bestrophin polypeptide in the plant or part thereof, where the bestrophin polypeptide is expressed in the plant or part thereof, and where when the plant is cultivated under ambient carbon dioxide conditions, the yield, growth rate, or biomass is greater than from a corresponding wild-type (WT) plant or corresponding WT part thereof that does not overexpress the bestrophin polypeptide or the yield, growth rate, or biomass is substantially similar to the yield, growth rate, or biomass from the corresponding wild-type (WT) plant or corresponding WT part thereof that does not overexpress the bestrophin polypeptide cultivated under ambient carbon dioxide conditions. In some embodiments, the bestrophin polypeptide is localized to a chloroplast envelope or a chloroplast thylakoid membrane of at least one chloroplast of a plant cell. In some embodiments, the plant cell is a leaf mesophyll cell. In some embodiments, the polypeptide is expressed in at least 70% of leaf mesophyll cells of the plant.

In some aspects, the present disclosure relates to plants or parts thereof with increased nitrogen use efficiency containing at least one modified nucleic acid sequence with at least one coding sequence of a green algal bestrophin polypeptide in the plant or part thereof, where the bestrophin polypeptide is expressed in the plant or part thereof, and where when the plant is cultivated under ambient carbon dioxide conditions, the yield, growth rate, or biomass is greater than from a corresponding wild-type (WT) plant or corresponding WT part thereof that does not overexpress the bestrophin polypeptide or the yield, growth rate, or biomass is substantially similar to the yield, growth rate, or biomass from the corresponding wild-type (WT) plant or corresponding WT part thereof that does not overexpress the bestrophin polypeptide cultivated under ambient carbon dioxide conditions. In some embodiments, the bestrophin polypeptide is localized to a chloroplast envelope or a chloroplast thylakoid membrane of at least one chloroplast of a plant cell. In some embodiments, the plant cell is a leaf mesophyll cell. In some embodiments, the polypeptide is expressed in at least 70% of leaf mesophyll cells of the plant.

In some aspects, the present disclosure relates to plants or parts thereof with reduced photoinhibition containing at least one modified nucleic acid sequence with at least one coding sequence of a green algal bestrophin polypeptide in the plant or part thereof, where the bestrophin polypeptide is expressed in the plant or part thereof, and where when the plant is cultivated under ambient carbon dioxide conditions, the yield, growth rate, or biomass is greater than from a corresponding wild-type (WT) plant or corresponding WT part thereof that does not overexpress the bestrophin polypeptide or the yield, growth rate, or biomass is substantially similar to the yield, growth rate, or biomass from the corresponding wild-type (WT) plant or corresponding WT part thereof that does not overexpress the bestrophin polypeptide cultivated under ambient carbon dioxide conditions. In some embodiments, the bestrophin polypeptide is localized to a chloroplast envelope or a chloroplast thylakoid membrane of at least one chloroplast of a plant cell. In some embodiments, the plant cell is a leaf mesophyll cell. In some embodiments, the polypeptide is expressed in at least 70% of leaf mesophyll cells of the plant.

In some embodiments of any of the above embodiments the modified nucleic acid sequence is stably integrated into the nuclear genome of the plant. In some embodiments of any of the above embodiments, the at least one modified nucleic acid sequence additionally contains a second nucleic acid sequence encoding a signal peptide sequence or targeting sequence operably linked to the at least one coding sequence of a green algal bestrophin polypeptide, where expression of the signal peptide sequence or targeting sequence results in localization of the bestrophin polypeptide to a chloroplast envelope or chloroplast thylakoid membrane of at least one chloroplast of a plant cell.

In some embodiments of any of the above embodiments, the increased carbon use efficiency, increased water use efficiency, increased nitrogen use efficiency, or reduced photoinhibition is the result of expression of a polypeptide selected from the group of a first polypeptide with at least 70% sequence identity, at least 75% sequence identity, at least 80% sequence identity, at least 85% sequence identity, at least 90% sequence identity, or at least 95% sequence identity to SEQ ID NO:1, a second polypeptide with at least 70% sequence identity, at least 75% sequence identity, at least 80% sequence identity, at least 85% sequence identity, at least 90% sequence identity, or at least 95% sequence identity to SEQ ID NO:2, a third polypeptide with at least 70% sequence identity, at least 75% sequence identity, at least 80% sequence identity, at least 85% sequence identity, at least 90% sequence identity, or at least 95% sequence identity to SEQ ID NO:3, or any combination thereof. In some embodiments of any of the above embodiments, the increased carbon use efficiency, increased water use efficiency, increased nitrogen use efficiency, or reduced photoinhibition is the result of expression of a polypeptide selected from the group of a first polypeptide with at least 95% sequence identity to SEQ ID NO:1, a second polypeptide with at least 95% sequence identity to SEQ ID NO:2, a third polypeptide with at least 95% sequence identity to SEQ ID NO:3, or any combination thereof. In some embodiments of any of the above embodiments, the increase or provision of the ability for bicarbonate to cross a membrane is the result of expression of a polypeptide selected from the group of SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, or SEQ ID NO:111, preferably selected from the group of SEQ ID NO:10, SEQ ID NO:11, SEQ ID NO:15, SEQ ID NO:27, SEQ ID NO:28, SEQ ID NO:29, SEQ ID NO:42, SEQ ID NO:62, or SEQ ID NO:63.

In some embodiments of any of the above embodiments, the plant is cowpea (i.e., black-eyed pea, Vigna unguiculata), soybean (i.e., soya bean, Glycine max), cassava (i.e., manioc, Manihot esculenta), rice (i.e., Oryza sativa, Oryza glaberrima, Zizania spp.), wheat (i.e., common wheat, spelt, durum, Triticum aestivum, Triticum spelta, Triticum durum, Triticum spp.), barley (i.e., Hordeum vulgare), rye (i.e., Secale cereale), oat (i.e., Avena sativa), potato (i.e., Solanum tuberosum), tomato (i.e., Solanum lycopersicum), or another C3 crop plant. In some embodiments, the plant is tobacco (i.e., Nicotiana tabacum, Nicotiana edwardsonii, Nicotiana plumbagnifolia, Nicotiana longiflora) or Arabidopsis (i.e., rockcress, thale cress, Arabidopsis thaliana). In some embodiments of any of the above embodiments, the plant is not corn (i.e., maize, Zea mays), sorghum (i.e., durra, great millet, milo, Sorghum bicolor), sugarcane (i.e., sugar cane, Saccharum officinarum), millet (i.e., finger millet, common millet, pearl millet, foxtail millet, Eleusine coracana, Panicum miliaceum, Pennisetum glaucum, Setaria italica), switchgrass (i.e., tall panic grass, thatchgrass, Panicum virganum), or another C4 crop plant.

In some embodiments, the plant part of any of the above embodiments is a leaf, a stem, a root, a flower, a seed, a fruit, a cell, or a portion thereof. In some embodiments, the plant part is a fruit. In some embodiments, the plant part is a grain, a kernel, a bean, or a tuber.

In some aspects, the present disclosure relates to a pollen grain or an ovule of any of the above embodiments.

In some aspects, the present disclosure relates to a protoplast produced from any of the above embodiments.

In some aspects, the present disclosure relates to a tissue culture produced from protoplasts or cells of any of the above embodiments, where the cells or protoplasts are produced from one of the plant parts in the group of leaf, anther, pistil, stem, petiole, root, root tip, fruit, seed, flower, cotyledon, hypocotyl, embryo, or meristematic cell.

In some aspects, the present disclosure relates to a genetically altered seed containing one or more genetic alterations that increase or provide the ability for bicarbonate to cross a membrane. In some embodiments, the seed produces a plant with the ability for bicarbonate to cross a membrane from a plant cell cytoplasm into a stroma of at least a portion of the chloroplasts of the plant. In some embodiments, the seed produces a plant with the ability for bicarbonate to cross a membrane from a stroma into a lumen of at least a portion of the chloroplasts of the plant. In some embodiments, the plant expresses at least one green algal bestrophin polypeptide. In some embodiments, the green algal bestrophin polypeptide is selected from the group of SEQ ID NO:1, SEQ ID NO:2, SEQ ID NO:3, or any combination thereof. In some embodiments, the plant expresses at least one polypeptide selected from the group of a first polypeptide with at least 70% sequence identity, at least 75% sequence identity, at least 80% sequence identity, at least 85% sequence identity, at least 90% sequence identity, or at least 95% sequence identity to SEQ ID NO:1, a second polypeptide with at least 70% sequence identity, at least 75% sequence identity, at least 80% sequence identity, at least 85% sequence identity, at least 90% sequence identity, or at least 95% sequence identity to SEQ ID NO:2, a third polypeptide with at least 70% sequence identity, at least 75% sequence identity, at least 80% sequence identity, at least 85% sequence identity, at least 90% sequence identity, or at least 95% sequence identity to SEQ ID NO:3, or any combination thereof. In some embodiments, the plant expresses at least one polypeptide selected from the group of a first polypeptide with at least 95% sequence identity to SEQ ID NO:1, a second polypeptide with at least 95% sequence identity to SEQ ID NO:2, a third polypeptide with at least 95% sequence identity to SEQ ID NO:3, or any combination thereof. In some embodiments, the plant expresses a polypeptide selected from the group of SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, or SEQ ID NO:111, preferably selected from the group of SEQ ID NO:10, SEQ ID NO:11, SEQ ID NO:15, SEQ ID NO:27, SEQ ID NO:28, SEQ ID NO:29, SEQ ID NO:42, SEQ ID NO:62, or SEQ ID NO:63. In some embodiments, the polypeptide is localized to a chloroplast envelope or a chloroplast thylakoid membrane of at least one chloroplast of a plant cell. In some embodiments, the plant cell is a leaf mesophyll cell. In some embodiments, the polypeptide is expressed in at least 70% of leaf mesophyll cells of the plant. In some embodiments, the plant is cowpea, soybean, cassava, rice, soy, wheat, or other C3 crop plants. In some embodiments, the plant is not corn, sorghum, or other C4 crop plants.

In some embodiments of any of the above embodiments, the expression of endogenous carbonic anhydrases is modulated. In some embodiments, modulated expression may be increased expression, reduced expression, expression at a different location, or any combination thereof.

Certain aspects of the present disclosure relate to a method of producing a plant with increased carbon use efficiency, where the steps of the method are: a) introducing a genetic alteration to the plant resulting in the increase or provision of the ability for bicarbonate to cross a membrane from a plant cell cytoplasm into a stroma of at least a portion of the chloroplasts of the plant or the increase or provision of the ability for bicarbonate to cross a membrane from a stroma into a lumen of at least a portion of the chloroplasts of the plant, thereby increasing carbon use efficiency of the plant.

In some aspects, the present disclosure relates to a method of producing a plant with increased water use efficiency, where the steps of the method are: a) introducing a genetic alteration to the plant resulting in the increase or provision of the ability for bicarbonate to cross a membrane from a plant cell cytoplasm into a stroma of at least a portion of the chloroplasts of the plant or the increase or provision of the ability for bicarbonate to cross a membrane from a stroma into a lumen of at least a portion of the chloroplasts of the plant, thereby increasing water use efficiency of the plant.

In some aspects, the present disclosure relates to a method of producing a plant with increased nitrogen use efficiency, where the steps of the method are: a) introducing a genetic alteration to the plant resulting in the increase or provision of the ability for bicarbonate to cross a membrane from a plant cell cytoplasm into a stroma of at least a portion of the chloroplasts of the plant or the increase or provision of the ability for bicarbonate to cross a membrane from a stroma into a lumen of at least a portion of the chloroplasts of the plant, thereby increasing nitrogen use efficiency of the plant.

In some aspects, the present disclosure relates to a method of producing a plant with reduced photoinhibition, where the steps of the method are: a) introducing a genetic alteration to the plant resulting in the increase or provision of the ability for bicarbonate to cross a membrane from a plant cell cytoplasm into a stroma of at least a portion of the chloroplasts of the plant or the increase or provision of the ability for bicarbonate to cross a membrane from a stroma into a lumen of at least a portion of the chloroplasts of the plant, thereby reducing photoinhibition of the plant.

In some aspects, the present disclosure relates to a method of producing a plant with increased growth or productivity, where the steps of the method are: a) introducing a genetic alteration to the plant resulting in the increase or provision of the ability for bicarbonate to cross a membrane from a plant cell cytoplasm into a stroma of at least a portion of the chloroplasts of the plant or the increase or provision of the ability for bicarbonate to cross a membrane from a stroma into a lumen of at least a portion of the chloroplasts of the plant, thereby increasing growth or productivity of the plant.

In some embodiments of any of the above methods, the expression of endogenous carbonic anhydrases is modulated. In some embodiments, modulated expression may be increased expression, reduced expression, expression at a different location, or any combination thereof.

In some aspects, the present disclosure relates to a method of cultivating a plant with increased carbon use efficiency, where the steps of the method are: a) providing a seed with one or more genetic alterations that increase or provide an ability for bicarbonate to cross a membrane, wherein the seed produces a plant with the ability for bicarbonate to cross a membrane from a plant cell cytoplasm into a stroma of at least a portion of the chloroplasts of the plant or a plant with the ability for bicarbonate to cross a membrane from a stroma into a lumen of at least a portion of the chloroplasts of the plant; b) cultivating the plant under conditions wherein the ability for bicarbonate to cross the membrane increases carbon use efficiency as compared to a plant grown under the same conditions that lacks the one or more genetic alterations.

In some aspects, the present disclosure relates to a method of cultivating a plant with increased water use efficiency, where the steps of the method are: a) providing a seed with one or more genetic alterations that increase or provide an ability for bicarbonate to cross a membrane, wherein the seed produces a plant with the ability for bicarbonate to cross a membrane from a plant cell cytoplasm into a stroma of at least a portion of the chloroplasts of the plant or a plant with the ability for bicarbonate to cross a membrane from a stroma into a lumen of at least a portion of the chloroplasts of the plant; b) cultivating the plant under conditions wherein the ability for bicarbonate to cross the membrane increases water use efficiency as compared to a plant grown under the same conditions that lacks the one or more genetic alterations.

In some aspects, the present disclosure relates to a method of cultivating a plant with increased nitrogen use efficiency, where the steps of the method are: a) providing a seed with one or more genetic alterations that increase or provide an ability for bicarbonate to cross a membrane, wherein the seed produces a plant with the ability for bicarbonate to cross a membrane from a plant cell cytoplasm into a stroma of at least a portion of the chloroplasts of the plant or a plant with the ability for bicarbonate to cross a membrane from a stroma into a lumen of at least a portion of the chloroplasts of the plant; b) cultivating the plant under conditions wherein the ability for bicarbonate to cross the membrane increases nitrogen use efficiency as compared to a plant grown under the same conditions that lacks the one or more genetic alterations.

In some aspects, the present disclosure relates to a method of cultivating a plant with reduced photoinhibition, where the steps of the method are: a) providing a seed with one or more genetic alterations that increase or provide an ability for bicarbonate to cross a membrane, wherein the seed produces a plant with the ability for bicarbonate to cross a membrane from a plant cell cytoplasm into a stroma of at least a portion of the chloroplasts of the plant or a plant with the ability for bicarbonate to cross a membrane from a stroma into a lumen of at least a portion of the chloroplasts of the plant; b) cultivating the plant under conditions wherein the ability for bicarbonate to cross the membrane reduces photoinhibition as compared to a plant grown under the same conditions that lacks the one or more genetic alterations.

In some aspects, the present disclosure relates to a method of cultivating a plant with increased growth or productivity, where the steps of the method are: a) providing a seed with one or more genetic alterations that increase or provide an ability for bicarbonate to cross a membrane, wherein the seed produces a plant with the ability for bicarbonate to cross a membrane from a plant cell cytoplasm into a stroma of at least a portion of the chloroplasts of the plant or a plant with the ability for bicarbonate to cross a membrane from a stroma into a lumen of at least a portion of the chloroplasts of the plant; b) cultivating the plant under conditions wherein the ability for bicarbonate to cross the membrane increases growth or productivity as compared to a plant grown under the same conditions that lacks the one or more genetic alterations.

In some aspects, the present disclosure relates to a method of cultivating a plant with increased carbon use efficiency, where the steps of the method are: a) providing a tissue culture or protoplast with one or more genetic alterations that increase or provide an ability for bicarbonate to cross a membrane; b) regenerating the tissue culture or protoplast into a plantlet; c) growing the plantlet into a plant, wherein the plant has the ability for bicarbonate to cross a membrane from a plant cell cytoplasm into a stroma of at least a portion of the chloroplasts of the plant or the ability for bicarbonate to cross a membrane from a stroma into a lumen of at least a portion of the chloroplasts of the plant; d) transplanting the plant into conditions wherein the ability for bicarbonate to cross the membrane increases carbon use efficiency as compared to a plant grown under the same conditions that lacks the one or more genetic alterations.

In some aspects, the present disclosure relates to a method of cultivating a plant with increased water use efficiency, where the steps of the method are: a) providing a tissue culture or protoplast with one or more genetic alterations that increase or provide an ability for bicarbonate to cross a membrane; b) regenerating the tissue culture or protoplast into a plantlet; c) growing the plantlet into a plant, wherein the plant has the ability for bicarbonate to cross a membrane from a plant cell cytoplasm into a stroma of at least a portion of the chloroplasts of the plant or the ability for bicarbonate to cross a membrane from a stroma into a lumen of at least a portion of the chloroplasts of the plant; d) transplanting the plant into conditions wherein the ability for bicarbonate to cross the membrane increases water use efficiency as compared to a plant grown under the same conditions that lacks the one or more genetic alterations.

In some aspects, the present disclosure relates to a method of cultivating a plant with increased nitrogen use efficiency, where the steps of the method are: a) providing a tissue culture or protoplast with one or more genetic alterations that increase or provide an ability for bicarbonate to cross a membrane; b) regenerating the tissue culture or protoplast into a plantlet; c) growing the plantlet into a plant, wherein the plant has the ability for bicarbonate to cross a membrane from a plant cell cytoplasm into a stroma of at least a portion of the chloroplasts of the plant or the ability for bicarbonate to cross a membrane from a stroma into a lumen of at least a portion of the chloroplasts of the plant; d) transplanting the plant into conditions wherein the ability for bicarbonate to cross the membrane increases nitrogen use efficiency as compared to a plant grown under the same conditions that lacks the one or more genetic alterations.

In some aspects, the present disclosure relates to a method of cultivating a plant with reduced photoinhibition, where the steps of the method are: a) providing a tissue culture or protoplast with one or more genetic alterations that increase or provide an ability for bicarbonate to cross a membrane; b) regenerating the tissue culture or protoplast into a plantlet; c) growing the plantlet into a plant, wherein the plant has the ability for bicarbonate to cross a membrane from a plant cell cytoplasm into a stroma of at least a portion of the chloroplasts of the plant or the ability for bicarbonate to cross a membrane from a stroma into a lumen of at least a portion of the chloroplasts of the plant; d) transplanting the plant into conditions wherein the ability for bicarbonate to cross the membrane reduces photoinhibition as compared to a plant grown under the same conditions that lacks the one or more genetic alterations.

In some aspects, the present disclosure relates to a method of cultivating a plant with increased growth or productivity, where the steps of the method are: a) providing a tissue culture or protoplast with one or more genetic alterations that increase or provide an ability for bicarbonate to cross a membrane; b) regenerating the tissue culture or protoplast into a plantlet; c) growing the plantlet into a plant, wherein the plant has the ability for bicarbonate to cross a membrane from a plant cell cytoplasm into a stroma of at least a portion of the chloroplasts of the plant or the ability for bicarbonate to cross a membrane from a stroma into a lumen of at least a portion of the chloroplasts of the plant; d) transplanting the plant into conditions wherein the ability for bicarbonate to cross the membrane increases growth or productivity as compared to a plant grown under the same conditions that lacks the one or more genetic alterations.

In some embodiments of any of the above methods, the seed, tissue culture, or protoplast has one or more genetic alterations that modulate the expression of endogenous carbonic anhydrases. In some embodiments, modulated expression may be increased expression, reduced expression, expression at a different location, or any combination thereof.

In some embodiments of any of the above methods, the increase or provision of the ability for bicarbonate to cross a membrane is the result of the expression of at least one polypeptide selected from the group of a first polypeptide with at least 95% sequence identity to SEQ ID NO:1, a second polypeptide with at least 95% sequence identity to SEQ ID NO:2, a third polypeptide with at least 95% sequence identity to SEQ ID NO:3, or any combination thereof. In some embodiments of any of the above methods, the increase or provision of the ability for bicarbonate to cross a membrane is the result of the expression of at least one polypeptide from selected from the group of SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, or SEQ ID NO:111, preferably selected from the group of SEQ ID NO:10, SEQ ID NO: 11, SEQ ID NO:15, SEQ ID NO:27, SEQ ID NO:28, SEQ ID NO:29, SEQ ID NO:42, SEQ ID NO:62, or SEQ ID NO:63. In some embodiments of any of the above methods, the plant is cowpea, soybean, cassava, rice, soy, wheat, or other C3 crop plants. In some embodiments of any of the above methods, the plant is not corn, sorghum, or other C4 crop plants.

Certain aspects of the present disclosure relate to a genetically altered alga containing one or more genetic alterations resulting in the increase of the ability of the alga to transport bicarbonate into a lumen of a chloroplast of the alga. In some embodiments, the increase of bicarbonate transport ability is the result of overexpressing at least one green algal bestrophin polypeptide from the group of a first polypeptide with at least 70% sequence identity, at least 75% sequence identity, at least 80% sequence identity, at least 85% sequence identity, at least 90% sequence identity, or at least 95% sequence identity to SEQ ID NO:1, a second polypeptide with at least 70% sequence identity, at least 75% sequence identity, at least 80% sequence identity, at least 85% sequence identity, at least 90% sequence identity, or at least 95% sequence identity to SEQ ID NO:2, a third polypeptide with at least 70% sequence identity, at least 75% sequence identity, at least 80% sequence identity, at least 85% sequence identity, at least 90% sequence identity, or at least 95% sequence identity to SEQ ID NO:3, or any combination thereof. In some embodiments, the increase of bicarbonate transport ability is the result of overexpressing at least one green algal bestrophin polypeptide from the group of a first polypeptide with at least 95% sequence identity to SEQ ID NO:1, a second polypeptide with at least 95% sequence identity to SEQ ID NO:2, a third polypeptide with at least 95% sequence identity to SEQ ID NO:3, or any combination thereof. In some embodiments, the polypeptide is localized to a chloroplast thylakoid membrane. In some embodiments, the polypeptide is overexpressed at least when the alga is under <100 ppm carbon dioxide (<0.01% [v/v] CO₂ in air) conditions.

In some aspects, the present disclosure relates to a green alga or part thereof with increased bicarbonate transport containing a modified nucleic acid sequence containing the coding sequence of at least one green algal bestrophin polypeptide; wherein the bestrophin polypeptide is overexpressed; wherein the bestrophin polypeptide is localized to chloroplast thylakoid membranes; and wherein when the alga is cultivated under <100 ppm carbon dioxide (<0.01% [v/v] CO₂ in air) conditions, the yield, growth rate, or biomass is greater than from a corresponding wild-type (WT) alga or WT part thereof that that does not overexpress the at least one bestrophin polypeptide or the yield, growth rate, or biomass is substantially similar to the yield, growth rate, or biomass from the corresponding WT alga or corresponding WT part thereof that does not overexpress the at least one bestrophin polypeptide cultivated under <100 ppm carbon dioxide (<0.01% [v/v] CO₂ in air) conditions. In some embodiments, the at least one green algal bestrophin polypeptide is selected from the group of a first polypeptide with at least 95% sequence identity to SEQ ID NO:1, a second polypeptide with at least 95% sequence identity to SEQ ID NO:2, a third polypeptide with at least 95% sequence identity to SEQ ID NO:3, or any combination thereof.

In some aspects, the present disclosure relates to a green alga or part thereof with increased growth under <100 ppm carbon dioxide (<0.01% [v/v] CO₂ in air) conditions containing a modified nucleic acid sequence containing the coding sequence of at least one green algal bestrophin polypeptide; wherein the bestrophin polypeptide is overexpressed; wherein the bestrophin polypeptide is localized to chloroplast thylakoid membranes; and wherein when the alga is cultivated under <100 ppm carbon dioxide (<0.01% [v/v] CO₂ in air) conditions, the yield, growth rate, or biomass is greater than from a corresponding wild-type (WT) alga or WT part thereof that that does not overexpress the at least one bestrophin polypeptide or the yield, growth rate, or biomass is substantially similar to the yield, growth rate, or biomass from the corresponding WT alga or corresponding WT part thereof that does not overexpress the at least one bestrophin polypeptide cultivated under <100 ppm carbon dioxide (<0.01% [v/v] CO₂ in air) conditions. In some embodiments, the at least one green algal bestrophin polypeptide is selected from the group of a first polypeptide with at least 95% sequence identity to SEQ ID NO:1, a second polypeptide with at least 95% sequence identity to SEQ ID NO:2, a third polypeptide with at least 95% sequence identity to SEQ ID NO:3, or any combination thereof.

In some embodiments of any of the above algae, the green alga is selected from the group of Chlamydomonas reinhardtii, Chlamydomonas eustigma, Volvox carteri f. nagariensis, and Gonium pectorale.

In some aspects, the present disclosure relates to a method of producing an alga with increased carbon use efficiency, where the steps of the method are: a) introducing a genetic alteration to the alga comprising the increase of the ability to transport bicarbonate into a lumen of a chloroplast of the alga, thereby increasing carbon use efficiency of the alga. In some embodiments, the gain of transport ability is the result of overexpressing at least one green algal bestrophin polypeptide selected from the group of a first polypeptide with at least 70% sequence identity, at least 75% sequence identity, at least 80% sequence identity, at least 85% sequence identity, at least 90% sequence identity, or at least 95% sequence identity to SEQ ID NO:1, a second polypeptide with at least 70% sequence identity, at least 75% sequence identity, at least 80% sequence identity, at least 85% sequence identity, at least 90% sequence identity, or at least 95% sequence identity to SEQ ID NO:2, a third polypeptide with at least 70% sequence identity, at least 75% sequence identity, at least 80% sequence identity, at least 85% sequence identity, at least 90% sequence identity, or at least 95% sequence identity to SEQ ID NO:3, or any combination thereof. In some embodiments, the increase of bicarbonate transport ability is the result of overexpressing at least one green algal bestrophin polypeptide selected from the group of a first polypeptide with at least 95% sequence identity to SEQ ID NO:1, a second polypeptide with at least 95% sequence identity to SEQ ID NO:2, a third polypeptide with at least 95% sequence identity to SEQ ID NO:3, or any combination thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D show the similarity of the C. reinhardtii genes (BST1, BST2, and BST3) and proteins (BST1, BST2, and BST3) to each other, as well as the similarity of BST1-3 to other bestrophin family proteins. FIG. 1A shows a schematic representation of the BST1, BST2, and BST3 genes, where the tan boxes represent exons, the thin grey lines represent introns, the thick grey lines represent untranslated regions (UTRs), and the overlaid red lines (overlaid on exons and introns) indicate the common region shared by the three genes. FIG. 1B shows the location and orientation of the BST1, BST2, and BST3 genes on the C. reinhardtii genome. FIG. 1C shows an amino acid alignment of the BST1 (SEQ ID NO:1), BST2 (SEQ ID NO:2), and BST3 (SEQ ID NO:3) proteins, where the asterisks in the bottom row indicate amino acids that are identical in all three proteins. FIG. 1D shows a phylogenetic tree of the protein sequences of C. reinhardtii BST1, BST2 and BST3 homologs in vascular plants (group second from the top; highlighted in purple), non-vascular plants (group third from the top; highlighted in blue), diatoms (group fourth from the top; highlighted in teal), and green algae (group at bottom; highlighted in green).

FIGS. 2A-2B show transcript analysis of the C. reinhardtii genes BST1, BST2, and BST3. FIG. 2A shows semi-quantitative RT-PCR showing BST1-3 accumulation in low CO₂ (<0.04% CO₂ in air) vs. high CO₂ (5% (v/v) CO₂ in air) in wild-type strain D66 and cia5 cells. FIG. 2B shows a semi-quantitative RT-PCR time course showing the expression of BST1-3 in cDNA obtained from cells grown in high CO₂ (5% CO₂ (v/v) in air) and in cells switched to low CO₂ (<0.04% CO₂ in air) for 2 hours (2 h), 4 hours (4 h), 6 hours (6 h), or 12 hours (12 h). Actin was used as a loading control in both FIG. 2A and FIG. 2B, and the results shown are from one of two replicates.

FIGS. 3A-3B show confocal microscope fluorescent protein images indicating the localization of BST1-Venus, BST2-Venus, and BST3-Venus fusion proteins in C. reinhardtii. FIG. 3A shows the location of BST1-Venus, BST2-Venus, and BST3-Venus fusion protein expression (“Venus” column), the location of chloroplast thylakoids (“Chlorophyll” column), and the location of both relative to each other (“Merge” column) in C. reinhardtii. The scale bar is 5 m. FIG. 3B shows zoomed-in images of the pyrenoid from the “BST1” row in FIG. 3A. Arrows highlight where BST1-Venus fluorescence can be seen inside the pyrenoid in the thylakoid tubules that penetrate the pyrenoid. The scale bar is 1 m. The images for both FIG. 3A and FIG. 3B are representative images of multiple replicates.

FIG. 4 shows results of qRT-PCR analysis of BST1-3 expression in the RNAi knockdown strains bsti-1 and bsti-2 as compared to the wild type strain D66. The error bars indicate the standard error for three biological replicates.

FIGS. 5A-5C show results of growth phenotype analysis of the RNAi knockdown strain bsti-1, as well as pmp1 and cia3 mutant strains as compared to the wild type strain D66 under different pH and CO₂ conditions. The vertical dots represent three different cell concentrations; 10,000 cells, 5,000 cells and 2,500 cells. FIG. 5A shows results of growth phenotype analysis of the strains under very low CO₂ (0.01% CO₂ (v/v) in air) conditions and a pH of 7 or 8.4. FIG. 5B shows results of growth phenotype analysis of the strains under low CO₂ (0.04% CO₂ (v/v) in air) and a pH of 7 or 8.4. FIG. 5C shows results of growth phenotype analysis of the strains under high CO₂ (5% CO₂ (v/v) in air) conditions and a pH of 7 or 8.4. The growth phenotype analysis experiment was repeated three times, and the results shown are representative.

FIGS. 6A-6F show photosynthetic oxygen evolution activity of BST-RNAi lines 1 and 2, i.e., bsti-1 and bsti-2, and D66. FIG. 6A shows the K_{0.5} (Cᵢ) values (Cᵢ concentration needed for half maximum oxygen evolution) that were calculated from the O₂ evolution versus Cᵢ curves for bsti-1 and D66 acclimated to low CO₂ (<0.04% CO₂) for 12 hours at pH 8.4. FIG. 6B shows oxygen evolving activity measured at different Cᵢ amounts and at different pH values plotted as curves for bsti-1 and D66 acclimated to low CO₂ (<0.04% CO₂) for 12 hours at pH 8.4. FIG. 6C shows the K_{0.5} (Cᵢ) values (Cᵢ concentration needed for half maximum oxygen evolution) that were calculated from the O₂ evolution versus Cᵢ curves for bsti-1, bsti-2, and D66 acclimated to low CO₂ (<0.04% CO₂) for 12 hours at pH 7.8. The symbol “*” indicates that the differences in K_{0.5}(Cᵢ) was significant (P<0.05 by student t test). FIG. 6D shows oxygen evolving activity measured at different Cᵢ amounts and at different pH values plotted as curves for bsti-1, bsti-2, and D66 acclimated to low CO₂ (<0.04% CO₂) for 12 hours at pH 7.8. FIG. 6E shows the K_{0.5} (Cᵢ) values (Cᵢ concentration needed for half maximum oxygen evolution) that were calculated from the O₂ evolution versus Cᵢ curves for bsti-1 and D66 acclimated to high CO₂ (>5% CO₂) for 12 hours at pH 7.8. FIG. 6F shows oxygen evolving activity measured at different Cᵢ amounts and at different pH values plotted as curves for bsti-1 and D66 acclimated to high CO₂ (>5% CO₂) for 12 hours at pH 7.8. For FIGS. 6A-6F, triplicate runs were made at each Cᵢ concentration, and the error bars represent three biological replicates and are based on standard deviation. The Vmax of all the strains was set to 100% oxygen evolution activity.

FIGS. 7A-7D show inorganic carbon uptake of bsti-1 and D66. FIG. 7A shows a time course of intracellular (internal) Cᵢ accumulation at pH 7.8. FIG. 7B shows a time course of CO₂ (Cᵢ) fixation at pH 7.8. FIG. 7C shows a time course of intracellular Cᵢ accumulation at pH 8.4. FIG. 7D shows a time course of CO₂ fixation at pH 8.4. Cells were grown in elevated CO₂ (5% CO₂ in air) and then acclimated to low CO₂ (<0.04% CO₂) for 12 hours prior to the assays. Cells were harvested and depleted of endogenous Cᵢ before running the assays, and triplicate samples were run for each time point. The error bars in FIGS. 7A-7D represent three biological replicates.

FIGS. 8A-8C show that BST3 is knocked out in the mutant bst3 from the Chlamydomonas Library Project (CLiP). FIG. 8A shows a schematic of the BST3 gene, where exons are depicted as blue boxes, introns are depicted as black lines, and untranslated regions are depicted as purple boxes. The location of the insert is shown as a green triangle, and primers to detect the insert are shown as small black arrows. FIG. 8B shows the results of PCR reactions using the primers shown in FIG. 8A to confirm the location of the insert in bst3. Lane 1, BST3F and BST3R primers using WT strain D66 DNA as template; lane 2, CIB1F and BST3F primers using bst3 DNA as template; lane 3, CIB1R and BST3R primers using bst3 DNA as template; lane 4, BST3F and BST3R primers using bst3 DNA as template. The size difference between lane 1 and 4 shows that there is an 1800 bp cassette. FIG. 8C shows semi-quantitative RT-PCR showing BST1-3 accumulation in low CO₂ (<0.04% CO₂ in air) vs. high CO₂ (5% (v/v) CO₂ in air) in D66 and bst3 cells. As indicated by the red box, BST3 is not expressed in bst3 cells. Actin was used as a loading control.

FIGS. 9A-9D show measurements of growth and inorganic carbon affinity of the knockout strain bst3 as compared to the WT strain D66. FIG. 9A shows growth of bst3 and WT in pH 8.6 at low CO₂ (<0.04% CO₂) over six days, which was measured using OD₇₃₀. FIG. 9B shows growth of bst3 and WT in pH 8.6 in pH 8.6 at low CO₂ (<0.04% CO₂) over six days, which was measured using chlorophyll estimation at wavelengths 645 and 663. FIG. 9C shows oxygen evolving activity measured at pH 7.4 plotted as curves for bst3 and D66. Triplicate runs were made at each Cᵢ concentration. FIG. 9D shows the K_{0.5} (Cᵢ) values (Cᵢ concentration needed for half maximum oxygen evolution) that were calculated from the O₂ evolution versus Cᵢ curves for bst3 and D66 at pH 7.4.

FIGS. 10A-10D show the protein structural models for C. reinhardtii BST1, BST2, and BST3. FIG. 10A shows structural models for C. reinhardtii BST1-3 and Klebsiella pneumonia bestrophin (Kpbest) as monomers with conserved residues lining the selective pore highlighted in red and labeled. FIG. 10B shows the secondary structure of a BST1 homopentamer model colored with the local QMEAN score (a structure quality assessment scoring function). FIG. 10C shows the outline of the channel cavity drawn on the BST1 homopentamer model in black. The conserved residues from the BST1 model in FIG. 10A are depicted as red shapes extending into the channel cavity. FIG. 10D shows the calculated electrostatic potential on the BST1 homopentamer model. The electrostatic potentials are displayed in potential scale of −4 kT/e (red, negative) to +4 kT/e (blue, positive).

FIG. 11 shows the current C. reinhardtii CCM model for inorganic carbon (Cᵢ) transport. BST1, BST2, and BST3 are shown as bicarbonate-transporting channels (light purple, purple, and dark purple rectangles) localized to the chloroplast thylakoid membrane.

### DETAILED DESCRIPTION

The following description sets forth exemplary methods, parameters, and the like. It should be recognized, however, that such description is not intended as a limitation on the scope of the present disclosure but is instead provided as a description of exemplary embodiments.

### Genetically Altered Plants and Seeds

Certain aspects of the present disclosure relate to a genetically altered plant or part thereof containing one or more genetic alterations that increase or provide the ability for bicarbonate to cross a membrane from a plant cell cytoplasm into a stroma of at least a portion of the chloroplasts of the plant. In some aspects, the present disclosure relates to a genetically altered plant or part thereof containing one or more genetic alterations that increase or provide the ability for bicarbonate to cross a membrane from a stroma into a lumen of at least a portion of the chloroplasts of the plant. In some embodiments, the gain of the bicarbonate membrane crossing ability is the result of the expression of at least one green algal bestrophin polypeptide. In some embodiments, the gain of the bicarbonate membrane crossing ability is the result of the expression of two or three green algal bestrophin polypeptides. In some embodiments, the gain of the bicarbonate membrane crossing ability is the result of the expression of four or more (e.g., five, six, seven, eight, nine, ten) green algal bestrophin polypeptides. In some embodiments, the green algal bestrophin polypeptide is SEQ ID NO:1 (i.e., BESTROPHIN1, BST1, Cre16.g662600.t1.2), SEQ ID NO:2 (i.e., BESTROPHIN2, BST2, Cre16.g663400.t2.1), or SEQ ID NO:3 (i.e., BESTROPHIN3, BST3, Cre16.g663450.t1.2). In some embodiments, the increase or provision of the ability for bicarbonate to cross a membrane is the result of expression of at least one polypeptide selected from the group of a first polypeptide with at least 70% sequence identity, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or at least 95% sequence identity to SEQ ID NO:1, a second polypeptide with at least 70% sequence identity. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or at least 95% sequence identity to SEQ ID NO:2, a third polypeptide with at least 70% sequence identity. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or at least 95% sequence identity to SEQ ID NO:3, or any combination thereof. In some embodiments, the increase or provision of the ability for bicarbonate to cross a membrane is the result of expression of a polypeptide selected from the group of a first polypeptide with at least 95% sequence identity to SEQ ID NO:1, a second polypeptide with at least 95% sequence identity to SEQ ID NO:2, a third polypeptide with at least 95% sequence identity to SEQ ID NO:3, or any combination thereof.

The term “BST1”, and capitalization and italicized versions thereof, refer to the green algal gene and protein, as described herein. In some embodiments, this term may refer to the C. reinhardtii gene and protein, as described herein. In other embodiments, this term may refer to one or more homologs or orthologs of the gene and protein of any green algal species that also transport bicarbonate. In some embodiments, this term may refer to one or more paralogs of the gene and protein of any green algal species. In some embodiments, the green algal species is C. reinhardtii, Chlamydomonas eustigma, Volvox carteri f. nagariensis, or Gonium pectorale. SEQ ID NO:1 provides the C. reinhardtii BST1 protein. When indicated with all lower-case letters in italics, the mutant (e.g., knockout) version of the gene/protein is intended. In C. reinhardtii, the mutant version may be a single gene/protein. In other green algal species, the mutant version may be one, some, or all homologs, orthologs, and/or paralogs of the genes/proteins.

The term “BST2”, and capitalization and italicized versions thereof, refer to the green algal gene and protein, as described herein. In some embodiments, this term may refer to the C. reinhardtii gene and protein, as described herein. In other embodiments, this term may refer to one or more homologs or orthologs of the gene and protein of any green algal species that also transport bicarbonate. In some embodiments, this term may refer to one or more paralogs of the gene and protein of any green algal species. In some embodiments, the green algal species is C. reinhardtii, Chlamydomonas eustigma, Volvox carteri f. nagariensis, or Gonium pectorale. SEQ ID NO:2 provides the C. reinhardtii BST2 protein. When indicated with all lower-case letters in italics, the mutant (e.g., knockout) version of the gene/protein is intended. In C. reinhardtii, the mutant version may be a single gene/protein. In other green algal species, the mutant version may be one, some, or all homologs, orthologs, and/or paralogs of the genes/proteins.

The term “BST3”, and capitalization and italicized versions thereof, refer to the green algal gene and protein, as described herein. In some embodiments, this term may refer to the C. reinhardtii gene and protein, as described herein. In other embodiments, this term may refer to one or more homologs or orthologs of the gene and protein of any green algal species that also transport bicarbonate. In some embodiments, this term may refer to one or more paralogs of the gene and protein of any green algal species. In some embodiments, the green algal species is C. reinhardtii, Chlamydomonas eustigma, Volvox carteri f. nagariensis, or Gonium pectorale. SEQ ID NO:3 provides the C. reinhardtii BST3 protein. When indicated with all lower-case letters in italics, the mutant (e.g., knockout) version of the gene/protein is intended. In C. reinhardtii, the mutant version may be a single gene/protein. In other green algal species, the mutant version may be one, some, or all homologs, orthologs, and/or paralogs of the genes/proteins.

In some embodiments, the increase or provision of the ability for bicarbonate to cross a membrane is the result of expression of a polypeptide from, or a polypeptide with high percent identity to one of, the group SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, or SEQ ID NO:111, preferably selected from the group consisting of SEQ ID NO:10, SEQ ID NO: 11, SEQ ID NO:15, SEQ ID NO:27, SEQ ID NO:28, SEQ ID NO:29, SEQ ID NO:42, SEQ ID NO:62, or SEQ ID NO:63. The phrases “high percent identical”, “high percent identity”, or “high sequence identity” and grammatical variations thereof in the context of two polynucleotides or polypeptides, refers to two or more sequences or sub-sequences that have at least about 80%, identity, at least about 81%, 82%, 83%, 84%, 85%, 86%, 87%, 88%, 89%, 90%, 91%, 92%, 93%, 94%, 95%, 96%, 97%, 98%, 99%, or 100% nucleotide or amino acid identity, when compared and aligned for maximum correspondence, as measured using a sequence comparison algorithm or by visual inspection. In an exemplary embodiment, a high percent identity exists over a region of the sequences that is at least about 16 nucleotides or amino acids in length. In another exemplary embodiment, a high percent identity exists over a region of the sequences that is at least about 50 nucleotides or amino acids in length. In still another exemplary embodiment, a high percent identity exists over a region of the sequences that is at least about 100 nucleotides or amino acids or more in length. In one exemplary embodiment, the sequences are high percent identical over the entire length of the polynucleotide or polypeptide sequences.

In some embodiments, the polypeptide is localized to a chloroplast envelope or a thylakoid membrane of at least one chloroplast within a plant cell. In some embodiments, the polypeptide is able to move bicarbonate (HCO₃⁻) across a membrane. In some embodiments, the polypeptide is able to move chloride anions (Cl⁻) across a membrane. In some embodiments, the polypeptide is able to move both HCO₃⁻ and Cl⁻ across a membrane. In some embodiments, the polypeptide is able to move negatively charged ions across a membrane. In some embodiments, the plant cell is a leaf mesophyll cell. In some embodiments, the polypeptide is expressed in at least 70% of leaf mesophyll cells of the plant. In some embodiments, the polypeptide oligomerizes to form a pentamer. In some embodiments, the polypeptide oligomerizes to form a homopentamer. In some embodiments, the pentamer or homopentamer has an entry pocket with a predominantly negative hydrostatic potential and a selective pore with a neutral/positive charge. In some embodiments, the homopentamer transports negatively charged ions.

In some aspects, the present disclosure relates to plants or parts thereof with increased carbon use efficiency containing at least one modified nucleic acid sequence with at least one coding sequence of a green algal bestrophin polypeptide in the plant or part thereof, where the bestrophin polypeptide is expressed in the plant or part thereof, and where when the plant is cultivated under ambient carbon dioxide conditions, the yield, growth rate, or biomass is greater than from a corresponding wild-type (WT) plant or corresponding WT part thereof that does not overexpress the bestrophin polypeptide or the yield, growth rate, or biomass is substantially similar to the yield, growth rate, or biomass from the corresponding wild-type (WT) plant or corresponding WT part thereof that does not overexpress the bestrophin polypeptide cultivated under ambient carbon dioxide conditions. As used herein, the term “ambient carbon dioxide” refers to the carbon dioxide content of air without any added or removed CO₂. In some embodiments, ambient carbon dioxide conditions are carbon dioxide conditions of 400-500 ppm, 400-550 ppm, 400-600 ppm, 400-650 ppm, 400-700 ppm, 450-500 ppm, 450-550 ppm, 450-600 ppm, 450-650 ppm, 450-700 ppm, 500-550 ppm, 500-600 ppm, 500-650 ppm, 500-700 ppm, 550-600 ppm, 550-650 ppm, 550-700 ppm, 600-650 ppm, 600-700 ppm, or 650-700 ppm. As used herein, the term “carbon use efficiency” may refer to the proportion of carbon acquired from the environment that is incorporated into the biomass of the plant. Carbon use efficiency can be measured by any known method in the art (e.g., subtracting the amount of carbon lost through plant respiration from the total amount of carbon that is taken up by the plant, then dividing this value by the total amount of carbon that is taken up by the plant, etc.). In some embodiments, the bestrophin polypeptide is localized to a chloroplast envelope or a chloroplast thylakoid membrane of at least one chloroplast of a plant cell. In some embodiments, the plant cell is a leaf mesophyll cell. In some embodiments, the polypeptide is expressed in at least 70% of leaf mesophyll cells of the plant.

In some aspects, the present disclosure relates to plants or parts thereof with increased water use efficiency containing at least one modified nucleic acid sequence with at least one coding sequence of a green algal bestrophin polypeptide in the plant or part thereof, where the bestrophin polypeptide is expressed in the plant or part thereof, and where when the plant is cultivated under ambient carbon dioxide conditions, the yield, growth rate, or biomass is greater than from a corresponding wild-type (WT) plant or corresponding WT part thereof that does not overexpress the bestrophin polypeptide or the yield, growth rate, or biomass is substantially similar to the yield, growth rate, or biomass from the corresponding wild-type (WT) plant or corresponding WT part thereof that does not overexpress the bestrophin polypeptide cultivated under ambient carbon dioxide conditions. As used herein, the term “water use efficiency” refers to the ratio of carbon assimilation to water consumption in a plant. Measures of water use efficiency include, but are not limited to, intrinsic water use efficiency and instantaneous water use efficiency. Instantaneous water use efficiency may be calculated by determining the ratio between plant carbon assimilation and plant transpiration. Intrinsic water use efficiency may be calculated by determining the ratio between plant carbon assimilation and plant stomatal conductance. Measures of carbon assimilation can include, but are not limited to, plant photosynthetic rate, yield, and biomass. In some embodiments, the bestrophin polypeptide is localized to a chloroplast envelope or a chloroplast thylakoid membrane of at least one chloroplast of a plant cell. In some embodiments, the plant cell is a leaf mesophyll cell. In some embodiments, the polypeptide is expressed in at least 70% of leaf mesophyll cells of the plant.

In some aspects, the present disclosure relates to plants or parts thereof with increased nitrogen use efficiency containing at least one modified nucleic acid sequence with at least one coding sequence of a green algal bestrophin polypeptide in the plant or part thereof, where the bestrophin polypeptide is expressed in the plant or part thereof, and where when the plant is cultivated under ambient carbon dioxide conditions, the yield, growth rate, or biomass is greater than from a corresponding wild-type (WT) plant or corresponding WT part thereof that does not overexpress the bestrophin polypeptide or the yield, growth rate, or biomass is substantially similar to the yield, growth rate, or biomass from the corresponding wild-type (WT) plant or corresponding WT part thereof that does not overexpress the bestrophin polypeptide cultivated under ambient carbon dioxide conditions. As used herein, the term “nitrogen use efficiency” refers to the ratio of nitrogen that is used by a plant for metabolism to total nitrogen supplied to the plant. Nitrogen use efficiency can be measured by any known method in the art (e.g., ¹⁵N isotope labeling, agronomic efficiency, apparent nitrogen recovery). Sources of nitrogen supplied to the plant include, but are not limited to, nitrogen contained in soil, nitrogen supplied by nitrogen-fixing bacteria, and nitrogen contained in fertilizers. In some embodiments, the bestrophin polypeptide is localized to a chloroplast envelope or a chloroplast thylakoid membrane of at least one chloroplast of a plant cell. In some embodiments, the plant cell is a leaf mesophyll cell. In some embodiments, the polypeptide is expressed in at least 70% of leaf mesophyll cells of the plant.

In some aspects, the present disclosure relates to plants or parts thereof with reduced photoinhibition containing at least one modified nucleic acid sequence with at least one coding sequence of a green algal bestrophin polypeptide in the plant or part thereof, where the bestrophin polypeptide is expressed in the plant or part thereof, and where when the plant is cultivated under ambient carbon dioxide conditions, the yield, growth rate, or biomass is greater than from a corresponding wild-type (WT) plant or corresponding WT part thereof that does not overexpress the bestrophin polypeptide or the yield, growth rate, or biomass is substantially similar to the yield, growth rate, or biomass from the corresponding wild-type (WT) plant or corresponding WT part thereof that does not overexpress the bestrophin polypeptide cultivated under ambient carbon dioxide conditions. As used herein, the term “photoinhibition” refers to the light-induced reduction of photosynthetic production in a plant. Photoinhibition can be measured by any known method in the art (e.g., rate of light saturated oxygen evolution, ratio of variable to maximum levels of chlorophyll a fluorescence). In some embodiments, the bestrophin polypeptide is localized to a chloroplast envelope or a chloroplast thylakoid membrane of at least one chloroplast of a plant cell. In some embodiments, the plant cell is a leaf mesophyll cell. In some embodiments, the polypeptide is expressed in at least 70% of leaf mesophyll cells of the plant.

In some embodiments of any of the above embodiments the modified nucleic acid sequence is stably integrated into the nuclear genome of the plant. In some embodiments of any of the above embodiments, the at least one modified nucleic acid sequence additionally contains a second nucleic acid sequence encoding a signal peptide sequence or targeting sequence operably linked to the at least one coding sequence of a green algal bestrophin polypeptide, where expression of the signal peptide sequence or targeting sequence results in localization of the bestrophin polypeptide to a chloroplast envelope or chloroplast thylakoid membrane of at least one chloroplast of a plant cell. In some embodiments, the signal peptide sequence or targeting sequence is a leader sequence. In some embodiments, the signal peptide sequence or targeting sequence is any sequence known in the art to result in polypeptide expression in the chloroplast envelope or the chloroplast thylakoid membrane.

In some embodiments of any of the above embodiments, the increased carbon use efficiency, increased water use efficiency, increased nitrogen use efficiency, or reduced photoinhibition is the result of expression of a first polypeptide with at least 70% sequence identity, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or at least 95% sequence identity to SEQ ID NO:1, a second polypeptide with at least 70% sequence identity, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or at least 95% sequence identity to SEQ ID NO:2, a third polypeptide with at least 70% sequence identity, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or at least 95% sequence identity to SEQ ID NO:3, or any combination thereof. In some embodiments of any of the above embodiments, the increased carbon use efficiency, increased water use efficiency, increased nitrogen use efficiency, or reduced Photoinhibition is the result of expression of a first polypeptide with at least 95% sequence identity to SEQ ID NO:1, a second polypeptide with at least 95% sequence identity to SEQ ID NO:2, a third polypeptide with at least 95% sequence identity to SEQ ID NO:3, or any combination thereof.

In some embodiments of any of the above embodiments, the increase or provision of the ability for bicarbonate to cross a membrane is the result of expression of a polypeptide from the group SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, or SEQ ID NO:111, preferably selected from the group consisting of SEQ ID NO:10, SEQ ID NO: 11, SEQ ID NO:15, SEQ ID NO:27, SEQ ID NO:28, SEQ ID NO:29, SEQ ID NO:42, SEQ ID NO:62, or SEQ ID NO:63.

In some embodiments of any of the above embodiments, the plant is cowpea (i.e., black-eyed pea, Vigna unguiculata), soybean (i.e., soya bean, Glycine max), cassava (i.e., manioc, Manihot esculenta), rice (i.e., Oryza sativa, Oryza glaberrima, Zizania spp.), wheat (i.e., common wheat, spelt, durum, Triticum aestivum, Triticum spelta, Triticum durum, Triticum spp.), barley (i.e., Hordeum vulgare), rye (i.e., Secale cereale), oat (i.e., Avena sativa), potato (i.e., Solanum tuberosum), tomato (i.e., Solanum lycopersicum), or another C3 crop plant. In some embodiments, the plant is tobacco (i.e., Nicotiana tabacum, Nicotiana edwardsonii, Nicotiana plumbagnifolia, Nicotiana longiflora) or Arabidopsis (i.e., rockcress, thale cress, Arabidopsis thaliana). In some embodiments of any of the above embodiments, the plant is not corn (i.e., maize, Zea mays), sorghum (i.e., durra, great millet, milo, Sorghum bicolor), sugarcane (i.e., sugar cane, Saccharum officinarum), millet (i.e., finger millet, common millet, pearl millet, foxtail millet, Eleusine coracana, Panicum miliaceum, Pennisetum glaucum, Setaria italica), switchgrass (i.e., tall panic grass, thatchgrass, Panicum virganum), or another C4 crop plant.

In some embodiments, the plant part of any of the above embodiments is a leaf, a stem, a root, a flower, a seed, a fruit, a cell, or a portion thereof. In some embodiments, the plant part is a fruit. In some embodiments, the plant part is a grain, a kernel, a bean, or a tuber.

In some aspects, the present disclosure relates to a pollen grain or an ovule of any of the above embodiments.

In some aspects, the present disclosure relates to a protoplast produced from any of the above embodiments.

In some aspects, the present disclosure relates to a tissue culture produced from protoplasts or cells of any of the above embodiments, where the cells or protoplasts are produced from one of the plant parts in the group of leaf, anther, pistil, stem, petiole, root, root tip, fruit, seed, flower, cotyledon, hypocotyl, embryo, or meristematic cell.

In some aspects, the present disclosure relates to a genetically altered seed containing one or more genetic alterations that increase or provide the ability for bicarbonate to cross a membrane. In some embodiments, the seed produces a plant with the ability for bicarbonate to cross a membrane from a plant cell cytoplasm into a stroma of at least a portion of the chloroplasts of the plant. In some embodiments, the seed produces a plant with the ability for bicarbonate to cross a membrane from a stroma into a lumen of at least a portion of the chloroplasts of the plant. In some embodiments, the plant expresses at least one green algal bestrophin polypeptide. In some embodiments, the green algal bestrophin polypeptide is SEQ ID NO:1, SEQ ID NO:2, SEQ ID NO:3, or any combination thereof. In some embodiments, the plant expresses a first polypeptide with at least 70% sequence identity, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or at least 95% sequence identity to SEQ ID NO:1, a second polypeptide with at least 70% sequence identity, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or at least 95% sequence identity to SEQ ID NO:2, a third polypeptide with at least 70% sequence identity, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or at least 95% sequence identity to SEQ ID NO:3, or any combination thereof. In some embodiments, the plant expresses a first polypeptide with at least 95% sequence identity to SEQ ID NO:1, a second polypeptide with at least 95% sequence identity to SEQ ID NO:2, a third polypeptide with at least 95% sequence identity to SEQ ID NO:3, or any combination thereof. In some embodiments, the plant expresses a polypeptide from the group SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, or SEQ ID NO:111, preferably selected from the group consisting of SEQ ID NO:10, SEQ ID NO: 11, SEQ ID NO:15, SEQ ID NO:27, SEQ ID NO:28, SEQ ID NO:29, SEQ ID NO:42, SEQ ID NO:62, or SEQ ID NO:63. In some embodiments, the polypeptide is localized to a chloroplast envelope or a chloroplast thylakoid membrane of at least one chloroplast of a plant cell. In some embodiments, the plant cell is a leaf mesophyll cell. In some embodiments, the polypeptide is expressed in at least 70% of leaf mesophyll cells of the plant. In some embodiments of any of the above embodiments, the plant is cowpea (i.e., black-eyed pea, Vigna unguiculata), soybean (i.e., soya bean, Glycine max), cassava (i.e., manioc, Manihot esculenta), rice (i.e., Oryza sativa, Oryza glaberrima, Zizania spp.), wheat (i.e., common wheat, spelt, durum, Triticum aestivum, Triticum spelta, Triticum durum, Triticum spp.), barley (i.e., Hordeum vulgare), rye (i.e., Secale cereale), oat (i.e., Avena sativa), potato (i.e., Solanum tuberosum), tomato (i.e., Solanum lycopersicum), or another C3 crop plant. In some embodiments, the plant is tobacco (i.e., Nicotiana tabacum, Nicotiana edwardsonii, Nicotiana plumbagnifolia, Nicotiana longiflora) or Arabidopsis (i.e., rockcress, thale cress, Arabidopsis thaliana). In some embodiments of any of the above embodiments, the plant is not corn (i.e., maize, Zea mays), sorghum (i.e., durra, great millet, milo, Sorghum bicolor), sugarcane (i.e., sugar cane, Saccharum officinarum), millet (i.e., finger millet, common millet, pearl millet, foxtail millet, Eleusine coracana, Panicum miliaceum, Pennisetum glaucum, Setaria italica), switchgrass (i.e., tall panic grass, thatchgrass, Panicum virganum), or another C4 crop plant.

In some embodiments of any of the above embodiments, the expression of endogenous carbonic anhydrases is modulated. In some embodiments, modulated expression may be increased expression, reduced expression, expression at a different location, or any combination thereof.

### Methods of Producing and Cultivating Genetically Altered Plants

Certain aspects of the present disclosure relate to a method of producing a plant with increased carbon use efficiency, where the steps of the method are: a) introducing a genetic alteration to the plant resulting in the increase or provision of the ability for bicarbonate to cross a membrane from a plant cell cytoplasm into a stroma of at least a portion of the chloroplasts of the plant or the increase or provision of the ability for bicarbonate to cross a membrane from a stroma into a lumen of at least a portion of the chloroplasts of the plant, thereby increasing carbon use efficiency of the plant.

In some aspects, the present disclosure relates to a method of producing a plant with increased water use efficiency, where the steps of the method are: a) introducing a genetic alteration to the plant resulting in the increase or provision of the ability for bicarbonate to cross a membrane from a plant cell cytoplasm into a stroma of at least a portion of the chloroplasts of the plant or the increase or provision of the ability for bicarbonate to cross a membrane from a stroma into a lumen of at least a portion of the chloroplasts of the plant, thereby increasing water use efficiency of the plant.

In some aspects, the present disclosure relates to a method of producing a plant with increased nitrogen use efficiency, where the steps of the method are: a) introducing a genetic alteration to the plant resulting in the increase or provision of the ability for bicarbonate to cross a membrane from a plant cell cytoplasm into a stroma of at least a portion of the chloroplasts of the plant or the increase or provision of the ability for bicarbonate to cross a membrane from a stroma into a lumen of at least a portion of the chloroplasts of the plant, thereby increasing nitrogen use efficiency of the plant.

In some aspects, the present disclosure relates to a method of producing a plant with reduced photoinhibition, where the steps of the method are: a) introducing a genetic alteration to the plant resulting in the increase or provision of the ability for bicarbonate to cross a membrane from a plant cell cytoplasm into a stroma of at least a portion of the chloroplasts of the plant or the increase or provision of the ability for bicarbonate to cross a membrane from a stroma into a lumen of at least a portion of the chloroplasts of the plant, thereby reducing photoinhibition of the plant.

In some aspects, the present disclosure relates to a method of producing a plant with increased growth or productivity, where the steps of the method are: a) introducing a genetic alteration to the plant resulting in the increase or provision of the ability for bicarbonate to cross a membrane from a plant cell cytoplasm into a stroma of at least a portion of the chloroplasts of the plant or the increase or provision of the ability for bicarbonate to cross a membrane from a stroma into a lumen of at least a portion of the chloroplasts of the plant, thereby increasing growth or productivity of the plant. Measures of increased growth can include, but are not limited to, faster growth rate, larger plants, increased biomass, increased dry mass, increased shoot mass, and increased root mass. Measures of increased productivity can include, but are not limited to, higher crop yield, greater numbers of leaves, and fewer days until crop maturity.

In some embodiments of any of the above methods, the expression of endogenous carbonic anhydrases is modulated. In some embodiments, modulated expression may be increased expression, reduced expression, expression at a different location, or any combination thereof.

In some aspects, the present disclosure relates to a method of cultivating a plant with increased carbon use efficiency, where the steps of the method are: a) providing a seed with one or more genetic alterations that increase or provide an ability for bicarbonate to cross a membrane, wherein the seed produces a plant with the ability for bicarbonate to cross a membrane from a plant cell cytoplasm into a stroma of at least a portion of the chloroplasts of the plant or a plant with the ability for bicarbonate to cross a membrane from a stroma into a lumen of at least a portion of the chloroplasts of the plant; b) cultivating the plant under conditions wherein the ability for bicarbonate to cross the membrane increases carbon use efficiency as compared to a plant grown under the same conditions that lacks the one or more genetic alterations.

In some aspects, the present disclosure relates to a method of cultivating a plant with increased water use efficiency, where the steps of the method are: a) providing a seed with one or more genetic alterations that increase or provide an ability for bicarbonate to cross a membrane, wherein the seed produces a plant with the ability for bicarbonate to cross a membrane from a plant cell cytoplasm into a stroma of at least a portion of the chloroplasts of the plant or a plant with the ability for bicarbonate to cross a membrane from a stroma into a lumen of at least a portion of the chloroplasts of the plant; b) cultivating the plant under conditions wherein the ability for bicarbonate to cross the membrane increases water use efficiency as compared to a plant grown under the same conditions that lacks the one or more genetic alterations.

In some aspects, the present disclosure relates to a method of cultivating a plant with increased nitrogen use efficiency, where the steps of the method are: a) providing a seed with one or more genetic alterations that increase or provide an ability for bicarbonate to cross a membrane, wherein the seed produces a plant with the ability for bicarbonate to cross a membrane from a plant cell cytoplasm into a stroma of at least a portion of the chloroplasts of the plant or a plant with the ability for bicarbonate to cross a membrane from a stroma into a lumen of at least a portion of the chloroplasts of the plant; b) cultivating the plant under conditions wherein the ability for bicarbonate to cross the membrane increases nitrogen use efficiency as compared to a plant grown under the same conditions that lacks the one or more genetic alterations.

In some aspects, the present disclosure relates to a method of cultivating a plant with reduced photoinhibition, where the steps of the method are: a) providing a seed with one or more genetic alterations that increase or provide an ability for bicarbonate to cross a membrane, wherein the seed produces a plant with the ability for bicarbonate to cross a membrane from a plant cell cytoplasm into a stroma of at least a portion of the chloroplasts of the plant or a plant with the ability for bicarbonate to cross a membrane from a stroma into a lumen of at least a portion of the chloroplasts of the plant; b) cultivating the plant under conditions wherein the ability for bicarbonate to cross the membrane reduces photoinhibition as compared to a plant grown under the same conditions that lacks the one or more genetic alterations.

In some aspects, the present disclosure relates to a method of cultivating a plant with increased growth or productivity, where the steps of the method are: a) providing a seed with one or more genetic alterations that increase or provide an ability for bicarbonate to cross a membrane, wherein the seed produces a plant with the ability for bicarbonate to cross a membrane from a plant cell cytoplasm into a stroma of at least a portion of the chloroplasts of the plant or a plant with the ability for bicarbonate to cross a membrane from a stroma into a lumen of at least a portion of the chloroplasts of the plant; b) cultivating the plant under conditions wherein the ability for bicarbonate to cross the membrane increases growth or productivity as compared to a plant grown under the same conditions that lacks the one or more genetic alterations.

In some aspects, the present disclosure relates to a method of cultivating a plant with increased carbon use efficiency, where the steps of the method are: a) providing a tissue culture or protoplast with one or more genetic alterations that increase or provide an ability for bicarbonate to cross a membrane; b) regenerating the tissue culture or protoplast into a plantlet; c) growing the plantlet into a plant, wherein the plant has the ability for bicarbonate to cross a membrane from a plant cell cytoplasm into a stroma of at least a portion of the chloroplasts of the plant or the ability for bicarbonate to cross a membrane from a stroma into a lumen of at least a portion of the chloroplasts of the plant; d) transplanting the plant into conditions wherein the ability for bicarbonate to cross the membrane increases carbon use efficiency as compared to a plant grown under the same conditions that lacks the one or more genetic alterations.

In some aspects, the present disclosure relates to a method of cultivating a plant with increased water use efficiency, where the steps of the method are: a) providing a tissue culture or protoplast with one or more genetic alterations that increase or provide an ability for bicarbonate to cross a membrane; b) regenerating the tissue culture or protoplast into a plantlet; c) growing the plantlet into a plant, wherein the plant has the ability for bicarbonate to cross a membrane from a plant cell cytoplasm into a stroma of at least a portion of the chloroplasts of the plant or the ability for bicarbonate to cross a membrane from a stroma into a lumen of at least a portion of the chloroplasts of the plant; d) transplanting the plant into conditions wherein the ability for bicarbonate to cross the membrane increases water use efficiency as compared to a plant grown under the same conditions that lacks the one or more genetic alterations.

In some aspects, the present disclosure relates to a method of cultivating a plant with increased nitrogen use efficiency, where the steps of the method are: a) providing a tissue culture or protoplast with one or more genetic alterations that increase or provide an ability for bicarbonate to cross a membrane; b) regenerating the tissue culture or protoplast into a plantlet; c) growing the plantlet into a plant, wherein the plant has the ability for bicarbonate to cross a membrane from a plant cell cytoplasm into a stroma of at least a portion of the chloroplasts of the plant or the ability for bicarbonate to cross a membrane from a stroma into a lumen of at least a portion of the chloroplasts of the plant; d) transplanting the plant into conditions wherein the ability for bicarbonate to cross the membrane increases nitrogen use efficiency as compared to a plant grown under the same conditions that lacks the one or more genetic alterations.

In some aspects, the present disclosure relates to a method of cultivating a plant with reduced photoinhibition, where the steps of the method are: a) providing a tissue culture or protoplast with one or more genetic alterations that increase or provide an ability for bicarbonate to cross a membrane; b) regenerating the tissue culture or protoplast into a plantlet; c) growing the plantlet into a plant, wherein the plant has the ability for bicarbonate to cross a membrane from a plant cell cytoplasm into a stroma of at least a portion of the chloroplasts of the plant or the ability for bicarbonate to cross a membrane from a stroma into a lumen of at least a portion of the chloroplasts of the plant; d) transplanting the plant into conditions wherein the ability for bicarbonate to cross the membrane reduces photoinhibition as compared to a plant grown under the same conditions that lacks the one or more genetic alterations.

In some aspects, the present disclosure relates to a method of cultivating a plant with increased growth or productivity, where the steps of the method are: a) providing a tissue culture or protoplast with one or more genetic alterations that increase or provide an ability for bicarbonate to cross a membrane; b) regenerating the tissue culture or protoplast into a plantlet; c) growing the plantlet into a plant, wherein the plant has the ability for bicarbonate to cross a membrane from a plant cell cytoplasm into a stroma of at least a portion of the chloroplasts of the plant or the ability for bicarbonate to cross a membrane from a stroma into a lumen of at least a portion of the chloroplasts of the plant; d) transplanting the plant into conditions wherein the ability for bicarbonate to cross the membrane increases growth or productivity as compared to a plant grown under the same conditions that lacks the one or more genetic alterations.

In some embodiments of any of the above methods, the seed, tissue culture, or protoplast has one or more genetic alterations that modulate the expression of endogenous carbonic anhydrases. In some embodiments, modulated expression may be increased expression, reduced expression, expression at a different location, or any combination thereof.

In some embodiments of any of the above methods, the increase or provision of the ability for bicarbonate to cross a membrane is the result of the expression of at least one polypeptide selected from the group of a first polypeptide with at least 95% sequence identity to SEQ ID NO:1, a second polypeptide with at least 95% sequence identity to SEQ ID NO:2, a third polypeptide with at least 95% sequence identity to SEQ ID NO:3, or any combination thereof. of any of the above methods, the increase or provision of the ability for bicarbonate to cross a membrane is the result of the expression of at least one polypeptide selected from the group of SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, or SEQ ID NO:111, preferably selected from the group consisting of SEQ ID NO:10, SEQ ID NO: 11, SEQ ID NO:15, SEQ ID NO:27, SEQ ID NO:28, SEQ ID NO:29, SEQ ID NO:42, SEQ ID NO:62, or SEQ ID NO:63. In some embodiments, the polypeptide is localized to a chloroplast envelope or a chloroplast thylakoid membrane of at least one chloroplast of a plant cell. In some embodiments, the plant cell is a leaf mesophyll cell. In some embodiments, the polypeptide is expressed in at least 70% of leaf mesophyll cells of the plant. In some embodiments of any of the above embodiments, the plant is cowpea (i.e., black-eyed pea, Vigna unguiculata), soybean (i.e., soya bean, Glycine max), cassava (i.e., manioc, Manihot esculenta), rice (i.e., Oryza sativa, Oryza glaberrima, Zizania spp.), wheat (i.e., common wheat, spelt, durum, Triticum aestivum, Triticum spelta, Triticum durum, Triticum spp.), barley (i.e., Hordeum vulgare), rye (i.e., Secale cereale), oat (i.e., Avena sativa), potato (i.e., Solanum tuberosum), tomato (i.e., Solanum lycopersicum), or another C3 crop plant. In some embodiments, the plant is tobacco (i.e., Nicotiana tabacum, Nicotiana edwardsonii, Nicotiana plumbagnifolia, Nicotiana longiflora) or Arabidopsis (i.e., rockcress, thale cress, Arabidopsis thaliana). In some embodiments of any of the above embodiments, the plant is not corn (i.e., maize, Zea mays), sorghum (i.e., durra, great millet, milo, Sorghum bicolor), sugarcane (i.e., sugar cane, Saccharum officinarum), millet (i.e., finger millet, common millet, pearl millet, foxtail millet, Eleusine coracana, Panicum miliaceum, Pennisetum glaucum, Setaria italica), switchgrass (i.e., tall panic grass, thatchgrass, Panicum virganum), or another C4 crop plant.

### Genetically Altered Algae

Certain aspects of the present disclosure relate to a genetically altered alga containing one or more genetic alterations resulting in the increase of the ability of the alga to transport bicarbonate into a lumen of a chloroplast of the alga. In some embodiments, the increase of bicarbonate transport ability is the result of overexpressing at least one green algal bestrophin polypeptide selected from the group of a first polypeptide with at least 70% sequence identity, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or at least 95% sequence identity to SEQ ID NO:1, a second polypeptide with at least 70% sequence identity, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or at least 95% sequence identity to SEQ ID NO:2, a third polypeptide with at least 70% sequence identity, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or at least 95% sequence identity to SEQ ID NO:3, or any combination thereof. In some embodiments, the increase of bicarbonate transport ability is the result of overexpressing at least one green algal bestrophin polypeptide selected from the group of a first polypeptide with at least 95% sequence identity to SEQ ID NO:1, a second polypeptide with at least 95% sequence identity to SEQ ID NO:2, a third polypeptide with at least 95% sequence identity to SEQ ID NO:3, or any combination thereof. In some embodiments, the polypeptide is localized to a chloroplast thylakoid membrane. In some embodiments, the polypeptide is overexpressed at least when the alga is under <100 ppm carbon dioxide (<0.01% [v/v] CO₂ in air) conditions.

In some aspects, the present disclosure relates to a green alga or part thereof with increased bicarbonate transport containing a modified nucleic acid sequence containing the coding sequence of at least one green algal bestrophin polypeptide; wherein the bestrophin polypeptide is overexpressed; wherein the bestrophin polypeptide is localized to chloroplast thylakoid membranes; and wherein when the alga is cultivated under <100 ppm carbon dioxide (<0.01% [v/v] CO₂ in air) conditions, the yield, growth rate, or biomass is greater than from a corresponding wild-type (WT) alga or WT part thereof that that does not overexpress the at least one bestrophin polypeptide or the yield, growth rate, or biomass is substantially similar to the yield, growth rate, or biomass from the corresponding WT alga or corresponding WT part thereof that does not overexpress the at least one bestrophin polypeptide cultivated under <100 ppm carbon dioxide (<0.01% [v/v] CO₂ in air) conditions. In some embodiments, the at least one green algal bestrophin polypeptide is selected from the group of a first polypeptide with at least 95% sequence identity to SEQ ID NO:1, a second polypeptide with at least 95% sequence identity to SEQ ID NO:2, a third polypeptide with at least 95% sequence identity to SEQ ID NO:3, or any combination thereof.

In some aspects, the present disclosure relates to a green alga or part thereof with increased growth under <100 ppm carbon dioxide (<0.01% [v/v] CO₂ in air) conditions containing a modified nucleic acid sequence containing the coding sequence of at least one green algal bestrophin polypeptide; wherein the bestrophin polypeptide is overexpressed; wherein the bestrophin polypeptide is localized to chloroplast thylakoid membranes; and wherein when the alga is cultivated under <100 ppm carbon dioxide (<0.01% [v/v] CO₂ in air) conditions, the yield, growth rate, or biomass is greater than from a corresponding wild-type (WT) alga or WT part thereof that that does not overexpress the at least one bestrophin polypeptide or the yield, growth rate, or biomass is substantially similar to the yield, growth rate, or biomass from the corresponding WT alga or corresponding WT part thereof that does not overexpress the at least one bestrophin polypeptide cultivated under <100 ppm carbon dioxide (<0.01% [v/v] CO₂ in air) conditions. In some embodiments, the at least one green algal bestrophin polypeptide is selected from the group of a first polypeptide with at least 95% sequence identity to SEQ ID NO:1, a second polypeptide with at least 95% sequence identity to SEQ ID NO:2, a third polypeptide with at least 95% sequence identity to SEQ ID NO:3, or any combination thereof.

In some embodiments of any of the above algae, the green alga is selected from the group of Chlamydomonas reinhardtii, Chlamydomonas eustigma, Volvox carteri f. nagariensis, and Gonium pectorale.

### Methods of Producing Genetically Altered Algae

In some aspects, the present disclosure relates to a method of producing an alga with increased carbon use efficiency, where the steps of the method are: a) introducing a genetic alteration to the alga comprising the increase of the ability to transport bicarbonate into a lumen of a chloroplast of the alga, thereby increasing carbon use efficiency of the alga. In some embodiments, the gain of transport ability is the result of overexpressing at least one green algal bestrophin polypeptide selected from the group of a first polypeptide with at least 70% sequence identity, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or at least 95% sequence identity to SEQ ID NO:1, a second polypeptide with at least 70% sequence identity, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or at least 95% sequence identity to SEQ ID NO:2, a third polypeptide with at least 70% sequence identity, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or at least 95% sequence identity to SEQ ID NO:3, or any combination thereof. In some embodiments, the increase of bicarbonate transport ability is the result of overexpressing at least one green algal bestrophin polypeptide selected from the group of a first polypeptide with at least 95% sequence identity to SEQ ID NO:1, a second polypeptide with at least 95% sequence identity to SEQ ID NO:2, a third polypeptide with at least 95% sequence identity to SEQ ID NO:3, or any combination thereof.

### Molecular Biological Methods to Produce Transgenic Plants and Plant Cells

One embodiment of the present invention provides a plant or plant cell comprising one or more modified plant genes and/or introduced genes. For example, the present disclosure provides transgenic plants with an increased or provided ability for bicarbonate to cross a membrane from a plant cell cytoplasm from a plant cell cytoplasm into a stroma of at least a portion of the chloroplasts of the plant. Further, the present disclosure provides transgenic plants with an increased or provided ability for bicarbonate to cross a membrane from a stroma into a lumen of at least a portion of the chloroplasts of the plant. In addition, the present disclosure provides transgenic plants with at least one modified nucleic acid sequence containing at least one coding sequence of a green algal bestrophin polypeptide. Modulated expression of other genetic elements (e.g., endogenous carbonic anhydrases) is also contemplated and described herein.

Transformation and generation of genetically altered monocotyledonous and dicotyledonous plant cells is well known in the art. See, e.g., Weising, et al., Ann. Rev. Genet. 22:421-477 (1988); U.S. Pat. No. 5,679,558; Agrobacterium Protocols, ed: Gartland, Humana Press Inc. (1995); and Wang, et al. Acta Hort. 461:401-408 (1998). The choice of method varies with the type of plant to be transformed, the particular application and/or the desired result. The appropriate transformation technique is readily chosen by the skilled practitioner.

Any methodology known in the art to delete, insert or otherwise modify the cellular DNA (e.g., genomic DNA and organelle DNA) can be used in practicing the inventions disclosed herein. For example, a disarmed Ti plasmid, containing a genetic construct for deletion or insertion of a target gene, in Agrobacterium tumefaciens can be used to transform a plant cell, and thereafter, a transformed plant can be regenerated from the transformed plant cell using procedures described in the art, for example, in EP 0116718, EP 0270822, PCT publication WO 84/02913 and published European Patent application (“EP”) 0242246. Ti-plasmid vectors each contain the gene between the border sequences, or at least located to the left of the right border sequence, of the T-DNA of the Ti-plasmid. Of course, other types of vectors can be used to transform the plant cell, using procedures such as direct gene transfer (as described, for example in EP 0233247), pollen mediated transformation (as described, for example in EP 0270356, PCT publication WO 85/01856, and U.S. Pat. No. 4,684,611), plant RNA virus-mediated transformation (as described, for example in EP 0 067 553 and U.S. Pat. No. 4,407,956), liposome-mediated transformation (as described, for example in U.S. Pat. No. 4,536,475), and other methods such as the methods for transforming certain lines of corn (e.g., U.S. Pat. No. 6,140,553; Fromm et al., Bio/Technology (1990) 8, 833 839); Gordon-Kamm et al., The Plant Cell, (1990) 2, 603 618) and rice (Shimamoto et al., Nature, (1989) 338, 274 276; Datta et al., Bio/Technology, (1990) 8, 736 740) and the method for transforming monocots generally (PCT publication WO 92/09696). For cotton transformation, the method described in PCT patent publication WO 00/71733 can be used. For soybean transformation, reference is made to methods known in the art, e.g., Hinchee et al. (Bio/Technology, (1988) 6, 915) and Christou et al. (Trends Biotech, (1990) 8, 145) or the method of WO 00/42207.

Transgenic plants of the present invention can be used in a conventional plant breeding scheme to produce more transgenic plants with the same characteristics, or to introduce the genetic alteration(s) in other varieties of the same or related plant species. Seeds, which are obtained from the transformed plants, preferably contain the genetic alteration(s) as a stable insert in chromosomal or organelle DNA. Plants comprising the genetic alteration(s) in accordance with the invention include plants comprising, or derived from, root stocks of plants comprising the genetic alteration(s) of the invention, e.g., fruit trees or ornamental plants. Hence, any non-transgenic grafted plant parts inserted on a transformed plant or plant part are included in the invention.

Introduced genetic elements, whether in an expression vector or expression cassette, which result in the expression of an introduced gene will typically utilize a plant-expressible promoter. A ‘plant-expressible promoter’ as used herein refers to a promoter that ensures expression of the genetic alteration(s) of the invention in a plant cell. Examples of promoters directing constitutive expression in plants are known in the art and include: the strong constitutive 35S promoters (the “35S promoters”) of the cauliflower mosaic virus (CaMV), e.g., of isolates CM 1841 (Gardner et al., Nucleic Acids Res, (1981) 9, 2871 2887), CabbB S (Franck et al., Cell (1980) 21, 285 294) and CabbB JI (Hull and Howell, Virology, (1987) 86, 482 493); promoters from the ubiquitin family (e.g., the maize ubiquitin promoter of Christensen et al., Plant Mol Biol, (1992) 18, 675-689), the gos2 promoter (de Pater et al., The Plant J (1992) 2, 834-844), the emu promoter (Last et al., Theor Appl Genet, (1990) 81, 581-588), actin promoters such as the promoter described by An et al. (The Plant J, (1996) 10, 107), the rice actin promoter described by Zhang et al. (The Plant Cell, (1991) 3, 1155-1165); promoters of the Cassava vein mosaic virus (WO 97/48819, Verdaguer et al. (Plant Mol Biol, (1998) 37, 1055-1067), the pPLEX series of promoters from Subterranean Clover Stunt Virus (WO 96/06932, particularly the S4 or S7 promoter), an alcohol dehydrogenase promoter, e.g., pAdh1S (GenBank accession numbers X04049, X00581), and the TR1′ promoter and the TR2′ promoter (the “TR1′ promoter” and “TR2′ promoter”, respectively) which drive the expression of the 1′ and 2′ genes, respectively, of the T DNA (Velten et al., EMBO J, (1984) 3, 2723 2730).

Alternatively, a plant-expressible promoter can be a tissue-specific promoter, i.e., a promoter directing a higher level of expression in some cells or tissues of the plant, e.g., in green tissues (such as the promoter of the PEP carboxylase). The plant PEP carboxylase promoter (Pathirana et al., Plant J, (1997) 12:293-304) has been described to be a strong promoter for expression in vascular tissue and is useful in one embodiment of the current invention. Alternatively, a plant-expressible promoter can also be a wound-inducible promoter, such as the promoter of the pea cell wall invertase gene (Zhang et al., Plant Physiol, (1996) 112:1111-1117). A ‘wound-inducible’ promoter as used herein means that upon wounding of the plant, either mechanically or by insect feeding, expression of the coding sequence under control of the promoter is significantly increased in such plant. These plant-expressible promoters can be combined with enhancer elements, they can be combined with minimal promoter elements, or can comprise repeated elements to ensure the expression profile desired.

In some embodiments, genetic elements to increase expression in plant cells can be utilized. For example, an intron at the 5′ end or 3′ end of an introduced gene, or in the coding sequence of the introduced gene, e.g., the hsp70 intron. Other such genetic elements can include, but are not limited to, promoter enhancer elements, duplicated or triplicated promoter regions, 5′ leader sequences different from another transgene or different from an endogenous (plant host) gene leader sequence, 3′ trailer sequences different from another transgene used in the same plant or different from an endogenous (plant host) trailer sequence.

An introduced gene of the present invention can be inserted in host cell DNA so that the inserted gene part is upstream (i.e., 5′) of suitable 3′ end transcription regulation signals (i.e., transcript formation and polyadenylation signals). This is preferably accomplished by inserting the gene in the plant cell genome (nuclear or chloroplast). Preferred polyadenylation and transcript formation signals include those of the nopaline synthase gene (Depicker et al., J. Molec Appl Gen, (1982) 1, 561-573), the octopine synthase gene (Gielen et al., EMBO J, (1984) 3:835 845), the SCSV or the Malic enzyme terminators (Schunmann et al., Plant Funct Biol, (2003) 30:453-460), and the T DNA gene 7 (Velten and Schell, Nucleic Acids Res, (1985) 13, 6981 6998), which act as 3′ untranslated DNA sequences in transformed plant cells. In some embodiments, one or more of the introduced genes are stably integrated into the nuclear genome. Stable integration is present when the nucleic acid sequence remains integrated into the nuclear genome and continues to be expressed (i.e., detectable mRNA transcript or protein is produced) throughout subsequent plant generations. Stable integration into the nuclear genome can be accomplished by any known method in the art (e.g., microparticle bombardment, Agrobacterium-mediated transformation, CRISPR/Cas9, electroporation of protoplasts, microinjection, etc.).

The term recombinant or modified nucleic acids refers to polynucleotides which are made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions.

As used herein, the terms “overexpression” and “upregulation” refer to increased expression (e.g., of mRNA, polypeptides, etc.) relative to expression in a wild type organism (e.g., plant, alga) as a result of genetic modification. In some embodiments, the increase in expression is a slight increase of about 10% more than expression in wild type. In some embodiments, the increase in expression is an increase of 50% or more (e.g., 60%, 70%, 80%, 100%, etc.) relative to expression in wild type. In some embodiments, an endogenous gene is overexpressed. In some embodiments, an exogenous gene is overexpressed by virtue of being expressed. Overexpression of a gene in plants or algae can be achieved through any known method in the art, including but not limited to, the use of constitutive promoters, inducible promoters, high expression promoters (e.g., PsaD promoter), enhancers, transcriptional and/or translational regulatory sequences, codon optimization, modified transcription factors, and/or mutant or modified genes that control expression of the gene to be overexpressed.

Where a recombinant nucleic acid is intended for expression, cloning, or replication of a particular sequence, DNA constructs prepared for introduction into a host cell will typically comprise a replication system (i.e. vector) recognized by the host, including the intended DNA fragment encoding a desired polypeptide, and can also include transcription and translational initiation regulatory sequences operably linked to the polypeptide-encoding segment. Additionally, such constructs can include cellular localization signals (e.g., chloroplast localization signals). In preferred embodiments, such DNA constructs are introduced into a host cell's genomic DNA, chloroplast DNA or mitochondrial DNA.

In some embodiments, a non-integrated expression system can be used to induce expression of one or more introduced genes. Expression systems (expression vectors) can include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides can also be included where appropriate from secreted polypeptides of the same or related species, which allow the protein to cross and/or lodge in cell membranes, cell wall, or be secreted from the cell.

Selectable markers useful in practicing the methodologies of the invention disclosed herein can be positive selectable markers. Typically, positive selection refers to the case in which a genetically altered cell can survive in the presence of a toxic substance only if the recombinant polynucleotide of interest is present within the cell. Negative selectable markers and screenable markers are also well known in the art and are contemplated by the present invention. One of skill in the art will recognize that any relevant markers available can be utilized in practicing the inventions disclosed herein.

Screening and molecular analysis of recombinant strains of the present invention can be performed utilizing nucleic acid hybridization techniques. Hybridization procedures are useful for identifying polynucleotides, such as those modified using the techniques described herein, with sufficient homology to the subject regulatory sequences to be useful as taught herein. The particular hybridization techniques are not essential to the subject invention. As improvements are made in hybridization techniques, they can be readily applied by one of skill in the art. Hybridization probes can be labeled with any appropriate label known to those of skill in the art. Hybridization conditions and washing conditions, for example temperature and salt concentration, can be altered to change the stringency of the detection threshold. See, e.g., Sambrook et al. (1989) vide infra or Ausubel et al. (1995) Current Protocols in Molecular Biology, John Wiley & Sons, NY, N.Y., for further guidance on hybridization conditions.

Additionally, screening and molecular analysis of genetically altered strains, as well as creation of desired isolated nucleic acids can be performed using Polymerase Chain Reaction (PCR). PCR is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al. (1985) Science 230:1350-1354). PCR is based on the enzymatic amplification of a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Because the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Thermus aquaticus, the amplification process can be completely automated. Other enzymes which can be used are known to those skilled in the art.

Nucleic acids and proteins of the present invention can also encompass homologues of the specifically disclosed sequences. Homology (e.g., sequence identity) can be 50%-100%. In some instances, such homology is greater than 80%, greater than 85%, greater than 90%, or greater than 95%. The degree of homology or identity needed for any intended use of the sequence(s) is readily identified by one of skill in the art. As used herein percent sequence identity of two nucleic acids is determined using an algorithm known in the art, such as that disclosed by Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:402-410. BLAST nucleotide searches are performed with the NBLAST program, score=100, wordlength=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (NBLAST and XBLAST) are used. See www.ncbi.nih.gov.

Preferred host cells are plant or algal cells. Recombinant host cells, in the present context, are those which have been genetically modified to contain an isolated nucleic molecule, contain one or more deleted or otherwise non-functional genes normally present and functional in the host cell, or contain one or more genes to produce at least one recombinant protein. The nucleic acid(s) encoding the protein(s) of the present invention can be introduced by any means known to the art which is appropriate for the particular type of cell, including without limitation, transformation, lipofection, electroporation or any other methodology known by those skilled in the art.

Having generally described this invention, the same will be better understood by reference to certain specific examples, which are included herein to further illustrate the invention and are not intended to limit the scope of the invention as defined by the claims.

### EXAMPLES

The present disclosure is described in further detail in the following examples which are not in any way intended to limit the scope of the disclosure as claimed. The attached figures are meant to be considered as integral parts of the specification and description of the disclosure. The following examples are offered to illustrate, but not to limit the claimed disclosure.

### Example 1: General Materials and Methods Used for Cell Culture and Growth

The following example describes the cell culture and growth conditions used in all of the following examples. Chlamydomonas reinhardtii cells were maintained on either Tris-acetate-phosphate (TAP) medium or yeast-acetate (YA) medium on petri plates. Before an experiment, cells were inoculated into minimal media (i.e., not containing a carbon source) and grown on high CO₂ (>5% CO₂ in air) to a density of 2-3×10⁶ cells mL⁻¹. These cells were then diluted into minimal media and grown at the pH and CO₂ concentrations indicated.

Chlamydomonas reinhardtii culture conditions were the same as described in Ma et al., Plant Physiol 156:884-896, 2011. The D66 strain (nit2⁻, cw15, mt⁺) was obtained from Dr. Rogene Schnell (University of Arkansas, Little Rock). CMJ030 (CC-4533; cw15, mt⁻) and bst3 (BST3 knockout LMJ.RY0402.089365) were obtained from the CLiP collection at the Chlamydomonas culture collection (Zhang et al., Plant Cell 26(4):1398-1409, 2014). Tris-Acetate-Phosphate (TAP) medium, yeast-acetate (YA) medium, and Minimal (MIN; without acetate, i.e., without a carbon source) were prepared according to Sueoka, PNAS 46:83-91, 1960. Both TAP and YA petri plates were prepared by adding 1.2% (w/v) agar, and C. reinhardtii cells were maintained on either type of petri plate. Cell cultures were initiated by inoculating colonies from TAP plates into 100 mL TAP liquid medium in Erlenmeyer flasks for mixotrophic growth. Cultures were grown to early log phase on continuous illumination (100 μmol m⁻² s⁻¹) and shaking for 48 hr. The early log phase TAP-grown cultures were harvested and washed with MIN medium, then re-suspended in MIN media and bubbled with high CO₂ (5% [v/v] CO₂ in air) to reach OD₇₃₀ between 0.2 and 0.3 (˜2-3×10⁶ cells mL⁻¹). For CCM induction, the cells were transferred to low CO₂ (<0.01% [v/v] CO₂ in air) bubbling for 12 hr or ambient CO₂ (0.04%-0.045% [v/v] CO₂ in air).

### Example 2: Phylogenetic Tree of Bestrophin Family Genes

The following example describes the construction of a phylogenetic tree depicting the evolutionary relationship of bestrophin family genes in a variety of photosynthetic organisms. In mammals, bestrophin proteins are known to conduct chloride and bicarbonate. In plants, most bestrophin proteins have not yet been characterized.

### Materials and Methods

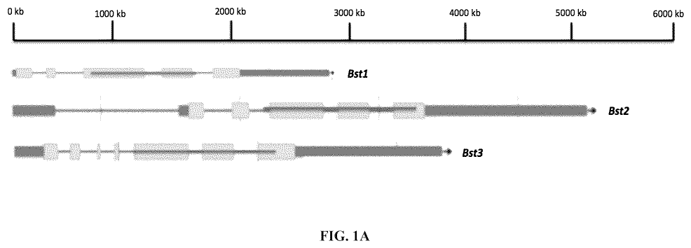

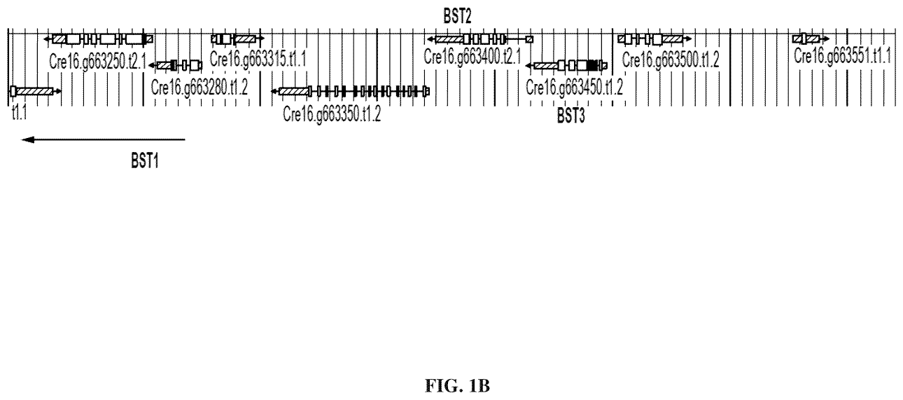

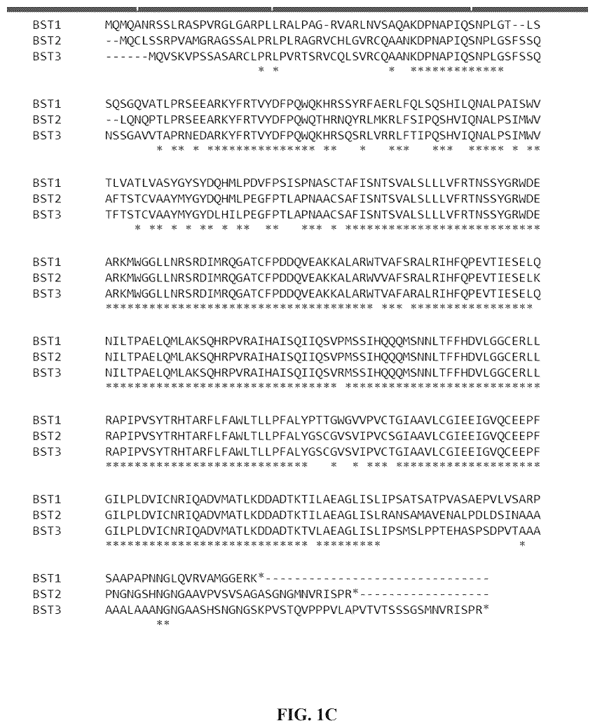

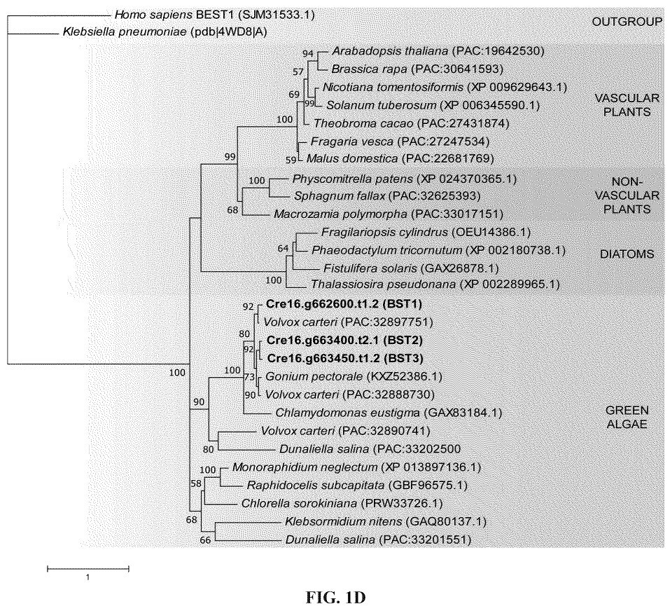

The three C. reinhardtii genes BESTROPHIN1 (BST1), BESTROPHIN2 (BST2), and BESTROPHIN3 (BST3) were chosen to be used in this tree for multiple reasons. For one, the BST1, BST2, and BST3 genes share a common region (see FIGS. 1A-1B). For another, the amino acid sequences of the BST1 (SEQ ID NO:1), BST2 (SEQ ID NO:2), and BST3 (SEQ ID NO:3) proteins are >80% identical to each other (see FIG. 1C). C. reinhardtii contains seven other predicted bestrophins, but none of these other bestrophins had more than a 45% identity with these three proteins. Finally, the three bestrophins chosen all were predicted to be targeted to the chloroplast.

Phylogenetic tree construction: Amino acid sequences for BST1 (Cre16.g662600.t1.2), BST2 (Cre16.g663400.t2.1), and BST3 (Cre16.g663450.t1.2) were BLASTED against NCBI Genbank (Benson et al., Nucleic Acids Res, 21(13):2963-2965, 1993) and Phytozome v12.1 (available at phytozome DOT jgi DOT doe DOT gov under pz/portal DOT html; Goodstein et al., Nucleic Acids Res 40(Database issue):D1178-1186, 2012). The top hits identified using these databases were downloaded. Additionally, amino acid sequences encoding Homo sapiens BEST1 (SJM31533.1) and Klebsiella pneumoniae bestrophin (pdb_4WD8_A) were downloaded from NCBI Genbank to be included as the outgroup in the phylogenetic analysis. A total of 63 initial sequences were aligned in Geneious 11.1.4 (Kearse et al., Bioinformatics 28(12):1647-1649, 2012) using a ClustalW (Thompson et al., Nucleic Acids Res 22(22):4673-4680, 1994) algorithm with the amino acid substitution matrix BLOSUM62 (Henikoff & Henikoff, PNAS 89(22):10915-10919, 1992). Duplicate sequences from the two databases (NCBI and Phytozome) and sequences with a pairwise percentage positive identity (BLOSUM62) of less than 70% were removed. The final alignment included 30 sequences and was manually trimmed to remove variable length ends from the sequences. The phylogenetic analysis was completed in MEGA X (Kumar et al., Comput Appl Biosci 10(2):189-191, 1994). The best Maximum Likelihood (ML) model for phylogenetic analysis of the alignment was calculated using the Model Selection function in MEGA X. A ML tree was constructed using the LG substitution model (Le & Gascuel, Mol Biol Evol 25(7):1307-1320, 2008) with Gamma distribution (5 discrete categories) and 500 bootstrap replicates (see FIG. 1D). Adobe Illustrator CC was used to format and shade the tree. A phylogenetic tree of the protein sequences of C. reinhardtii BST1, BST2 and BST3 homologs was constructed using maximum likelihood (ML).

### Results

FIG. 1A shows a schematic representation of the BST1, BST2, and BST3 genes. The tan boxes represent exons, the thin grey lines represent introns, the thick grey lines represent untranslated regions (UTRs), and the red lines overlaid on the gene schematic (overlaid on exons and introns) indicate the common region shared by the three genes. Further, the lengths of each of the three bestrophin genes is shown, with BST1 being the shortest at under 3000 kb, and BST2 being the longest at over 5000 kb. In FIG. 1B, the location and orientation of the BST1, BST2, and BST3 genes on the C. reinhardtii genome is shown. BST1 (Cre16.g662600), BST2 (Cre16.g663400) and BST3 (Cre16.g663450) are paralogous genes located within a 130 kbp region on the 16th chromosome of C. reinhardtii. In addition, this image depicts the location of BST1, BST2, and BST3 relative to each other. FIG. 1C depicts an amino acid alignment of the BST1, BST2, and BST3 proteins, where the asterisks in the bottom row indicate amino acids that are identical in all three proteins. The BST1, BST2, and BST3 proteins are >80% identical to each other.

FIG. 1D shows a phylogenetic tree representing the relationship among the protein sequences of C. reinhardtii BST1, BST2 and BST3 homologs in a variety of photosynthetic organisms. These organisms include vascular plants (group second from the top; highlighted in purple), non-vascular plants (group third from the top; highlighted in blue), diatoms (group fourth from the top; highlighted in teal), and green algae (group at bottom; highlighted in green). The C. reinhardtii bestrophin proteins are shown in bold within the green-highlighted green algal group. An outgroup, consisting of proteins from the non-photosynthetic organisms human (Homo sapiens) and bacteria (Klebsiella pneumoniae), is also shown (group at top: highlighted in pink). Sequence alignment of C. reinhardtii BST1, BST2 and BST3 with human Bestrophin 1 (BEST1) showed low sequence identity between BEST1 and BST1-3 (21-23%). The C. reinhardtii bestrophin proteins show considerable sequence similarity to each other, and to other green algal bestrophin proteins, which is depicted by their proximal location within the tree and the shallower branching pattern connecting these proteins. In particular, V. carteri and Chlamydomonas eustigma are very closely related to C. reinhardtii, while Dunaliella salina is more distantly related to C. reinhardtii. Diatom species (highlighted in teal) are very distantly related to C. reinhardtii, and in fact arose from a different endosymbiotic event; this distant relationship is clearly depicted by the deeper branching pattern connecting these proteins. The vascular and non-vascular plant proteins shown in FIG. 1D have low sequence similarity of only 30-35% to the C. reinhardtii bestrophin proteins, which is depicted by their more distant location and the deeper branching pattern connecting these proteins. For example, the thylakoid localized VCCN1 protein of Arabidopsis has about a 30% sequence identity with BST1-3. Although the other proteins used to construct this tree are also bestrophin family genes, they are only distantly related to the C. reinhardtii bestrophins. At the amino acid level, C. reinhardtii bestrophins have less than 50% identity with the bestrophin proteins found in higher plants or mammals. Further, the human bestrophin protein is sufficiently different from the C. reinhardtii bestrophins that it can be used as an outgroup, along with the bacterial K. pneumoniae bestrophin protein, to anchor the phylogenetic tree.

### Example 3: Bestrophins are Upregulated Under Low CO₂ and their Expression is Controlled by CIA5

CIA5 is a transcription factor that controls many CCM genes. In particular, CIA5 controls all of the known CCM transporters. The following example describes the analysis of bestrophin expression in a wild type (WT) D66 C. reinhardtii strain, and a cia5 mutant C. reinhardtii strain grown under either high CO₂ or low CO₂ conditions.

### Materials and Methods

Cell culture and growth conditions for C. reinhardtii cells were as described in Example 1. For the experiment, a WT D66 C. reinhardtii strain and a cia5 mutant C. reinhardtii strain were cultured under either high CO₂ (5% [v/v] CO₂ in air) or low CO₂ conditions (<0.04% [v/v] CO₂ in air, i.e., ambient CO₂ conditions).

RNA samples were obtained from high CO₂ and low CO₂ acclimated cultures of both C. reinhardtii strains. RNA extraction was carried out using the Trizol reagent (Invitrogen) according to manufacturer's instructions. 1 ug RNA per sample was used as template for cDNA, which was made using ProtoScript® First Strand cDNA Synthesis Kit (NEB) as per manufacturer's instructions. 100 ng RNA per sample was used to conduct qRTPCR using the Luna® Universal One-Step RT-qPCR Kit from NEB as per manufacturer's instructions using QuantStudio 6. Semi-quantitative RT-PCR was performed using primers specific for BST1, BST2, or BST3, and Actin primers were as a control (listed in Table 1). PCR products were analyzed using gel electrophoresis. The experiment was replicated twice.

TABLE 1
Primers used for RT-PCR analysis.

| Gene | Primer | |
|---|---|---|
| target | name | Primer sequence |
| BST1 | BST1 RT-F | GACACCAAGACCATCCTGGC |
| | | (SEQ ID NO: 112) |
| | BST1 RT-R | AACAGAACTGCAGAGGTCCCG |
| | | (SEQ ID NO: 113) |
| BST2 | BST2 RT-F | CGGTGCCCATGAGCTCC |
| | | (SEQ ID NO: 114) |
| | BST2 RT-R | GCCACTAACCGGCCCAA |
| | | (SEQ ID NO: 115) |
| BST3 | BST3 RT-F | AATCCCGTCCATGTCGCT |
| | | (SEQ ID NO: 116) |
| | BST3 RT-R | CGGCTTGTGAGGACCTCG |
| | | (SEQ ID NO: 117) |
| Actin | Actin RT-F | GCCAGAAGGACTCGTACGTT |
| | | (SEQ ID NO: 118) |
| | Actin RT-R | CGCCAGAGTCCAGCACGATA |
| | | (SEQ ID NO: 119) |

A time course analysis of BST1, BST2, and BST3 expression was also carried out. For this analysis, cells grown under high CO₂ were transferred to low CO₂ for 2 to 12 hours, and then RNA was extracted and cDNA was made as described above. This analysis was replicated twice.

### Results

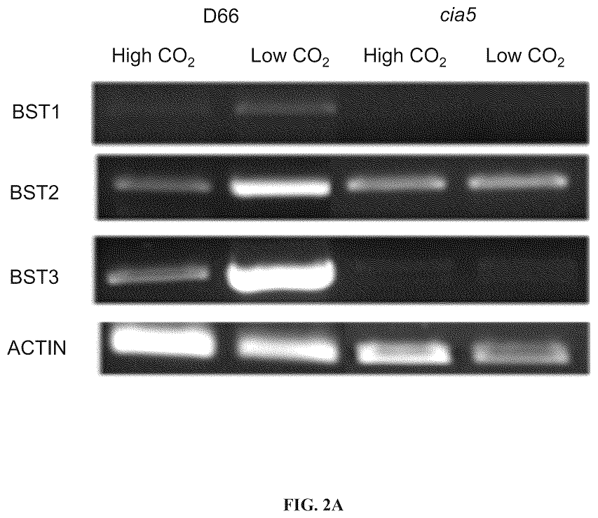

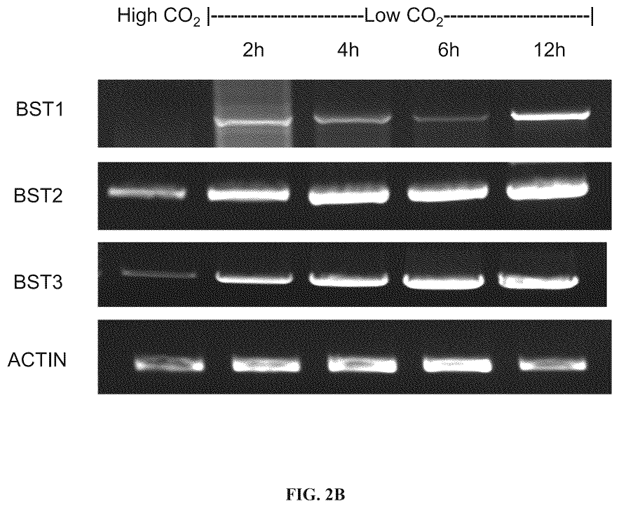

As shown in FIG. 2A, in D66 (WT), the expression levels of all three bestrophin genes were upregulated under low CO₂ conditions, as compared to high CO₂ conditions. In particular, BST3 expression was very low in cells grown under high CO₂ conditions, and much higher in cells grown under low CO₂ conditions. The increased intensity of the bands from the D66 cells grown in low CO₂ conditions as compared to the D66 cells grown in high CO₂ conditions clearly shows that all three genes are upregulated under low CO₂ conditions. In contrast to D66, the low CO₂ condition did not induce increased expression of BST1, BST2, or BST3 in cia5 mutants. Moreover, BST1 and BST3 were not expressed in the cia5 mutant under either condition, which is a similar transcriptional pattern to other CCM genes in the cia5 mutant (Xiang et al., PNAS 98(9):5341-5346, 2001; Fukuzawa et al., PNAS 98(9):5347-5352, 2001; Moroney et al., Plant Physiol 89(3):897-903, 1989). BST2 transcript levels in cia5 cells were the same in both high CO₂ and low CO₂ conditions, and showed no noticeable induction in low CO₂. This is in contrast to D66 cells, where BST2 transcript levels increase in low CO₂ conditions.

FIG. 2B shows the expression of the three BST genes in D66 at different time points after transfer from high CO₂ conditions to low CO₂ conditions. All three genes had increased transcript levels within 2 hours after the switch to low CO₂ and these levels of expression were maintained until at least 12 hours after induction. These results demonstrate that all three BST genes were upregulated under low CO₂ growth conditions known to induce CCM. Further, this upregulation was not observed in cia5 mutants, which suggests that the expression of the bestrophin genes is regulated by CIA5. Taken together, these results indicate a possible role in the CCM for the bestrophin genes.

### Example 4: Localization of Bestrophins in the Chloroplast

The current CCM model indicates that bicarbonate transporters at the chloroplast thylakoid membrane are necessary for bicarbonate uptake, but these transporters have not yet been identified. The following example describes the localization of chimeric bestrophin-Venus proteins within the C. reinhardtii cell. A fluorescence imaging approach was chosen to identify the specific location within the chloroplast, because computational analysis predicted chloroplast targeting on the basis of the leader sequences of the three bestrophin proteins, but not a specific location within the chloroplast.

### Materials and Methods

Fluorescence protein tagging: For this experiment, the coding sequences of BST1, BST2, or BST3 were fused with the coding sequence of Venus in order to generate fusion proteins BST1-Venus, BST2-Venus, and BST3-Venus. The BST1-3 genes were cloned and transformed into Chlamydomonas strain CC-4533 as in Mackinder et al., Cell 171(1):133-147 e114, 2017. Briefly, the open reading frames of BST1-3 genes were PCR amplified from genomic DNA (primers listed in Table 2) and cloned into pLM005 with C-terminal Venus-3×FLAG and a PSAD promoter through Gibson assembly. Three separate constructs were generated, one for each BST. PSAD is a high expression promoter which drives a nuclear gene encoding an abundant chloroplast protein located on the stromal side of photosystem I in C. reinhardtii (Fischer and Rochaix, 2001). For transformation, wild type cultures were grown to mid-log phase and concentrated to 2×10⁸ cells mL⁻¹. The suspension was mixed with the constructed plasmid linearized by EcoRV prior to electroporation. Then, the suspension was plated on TAP paramomycin (20 μg mL⁻¹) for selection. Three separate strains were generated: one expressing BST1-Venus, one expressing BST2-Venus, and one expressing BST3-Venus. Fluorescent colonies were identified using a Typhoon 8610 scanner. Laser settings for Venus were 532 nm for excitation and 555/20 for emission, and chlorophyll autofluorescence was excited at 633 nm with 670/30 emission.

TABLE 2
Primers used for generating Venus-tagged
fluorescent BST1-3 proteins.

| Primer name | Primer sequence |
|---|---|
| BST1F | GCTACTCACAACAAGCCCAGTT |
| | ATGCAGATGCAAGCAAACCGTTCGTC |
| | (SEQ ID NO: 120) |
| BST1R | GAGCCACCCAGATCTCCGTTCT |
| | TGCGCTCCCCACCCATGG |
| | (SEQ ID NO: 121) |
| BST2F | GCTACTCACAACAAGCCCAGTT |
| | ATGGCCACTGGTCAGACC |
| | (SEQ ID NO: 122) |
| BST2R | GAGCCACCCAGATCTCCGTTTC |
| | TCCTTGTCTCCGCAC |
| | (SEQ ID NO: 123) |
| BST3F | GCTACTCACAACAAGCCCAGTT |
| | ATGCAAGTCAGCAAGGTTCCCTCG |
| | (SEQ ID NO: 124) |
| BST3R | GAGCCACCCAGATCTCCGTT |
| | CCGGGGCGAGATGCGCAC |
| | (SEQ ID NO: 125) |

Confocal microscopy: Fluorescent images of the three generated strains were then taken using a confocal microscope in order to determine bestrophin protein localization. Identified fluorescent colonies were grown heterotrophically in TAP medium until reaching mid-log phase. Cultures were then harvested and re-suspended in Tris-minimal medium overnight prior to imaging. Images were captured with Laser-scanning microscope LSM880 (Zeiss) equipped with an Airyscan module using an x63 objectives with 1.4 NA. Argon lasers at 514 nm and 561 nm were used for excitation of Venus and chlorophyll respectively. Filters were set at 525-550 nm for the Venus emission and 620-670 nm for chlorophyll emission. Chlorophyll fluorescence was used to localize chloroplast thylakoid stacks in these strains, and an overlay of Venus and chlorophyll fluorescence was used to identify the location of bestrophin proteins relative to chloroplast thylakoid stacks. Multiple replicates were imaged.

### Results

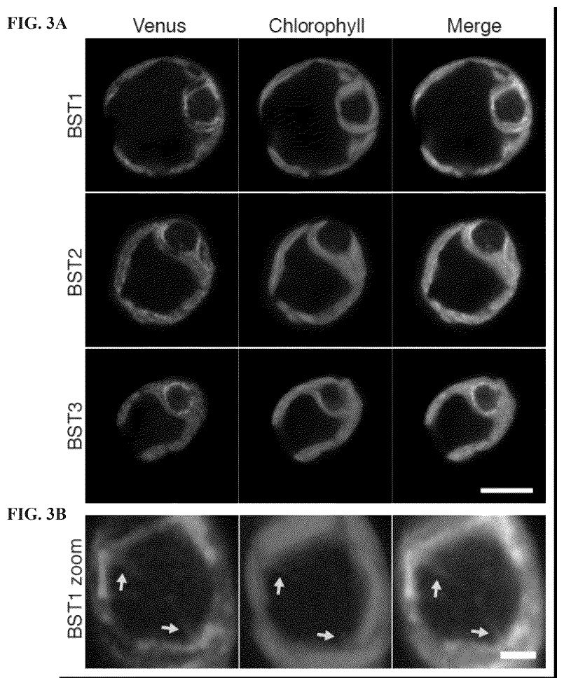

As shown in FIG. 3A, all three bestrophin fusion proteins presented non-homogenous signals within the chloroplast, indicated by Venus signals (shown in left column). When the Venus signals and the chlorophyll signals (shown in middle column) were merged, an overlap could be seen between the Venus signals and the thylakoid stacks in the chloroplast. All three BST-Venus fusion proteins localized to the thylakoid membranes of the chloroplast. In addition, the thylakoid tubules of the pyrenoid show the same overlap between the Venus signals and the chlorophyll signals. This is depicted clearly in FIG. 3B, which shows zoomed-in pyrenoid images of the BST1-Venus strain. The arrows indicate where BST1-Venus fluorescence can be seen inside the pyrenoid in the thylakoid tubules that penetrate the pyrenoid. These results both confirm the computational prediction that bestrophins are targeted to the chloroplast (based on bestrophin leader sequences), and strongly indicate a thylakoid localization for the bestrophin proteins.

### Example 5: Bestrophins are Required for C. reinhardtii Growth Under Low CO₂

The following example describes the effect of reducing the expression of the three bestrophins BST1, BST2, and BST3 on the growth of C. reinhardtii. The growth phenotype of the triple bestrophin RNAi knockdown strain bsti-1 was compared to the WT strain D66 as well as to the mutant strains cia3 and pmp1 under growth conditions varied in CO₂ concentration and pH. The two mutant strains have mutations in known CCM components; the cia3 strain has a mutation in CAH3, which is a thylakoid carbonic anhydrase, while the pmp1 strain has a mutation in LCIB/LCIC, which are stromal theta carbonic anhydrases.

### Materials and Methods

BST1, BST2 and BST3 knockdown using RNAi: The RNAi knockdown approach was chosen in order to target all three bestrophin genes simultaneously. At present, only a bst3 knockout strain is available from the Chlamydomonas Culture Collection; bst1 and bst2 knockout strains are not available. Moreover, as described in more detail in Example 7, a bst3 knockout strain was tested under low CO₂ conditions, and found to grow normally. This means both that only the bst3 knockout strain has been tested, and that the material to generate a triple knockout strain is not currently available. The RNAi knockdown approach was therefore considered the best option.

Artificial microRNA construct for the knock-down of the BST proteins were made using the protocol of Molnar, et al. Plant J 58(1):165-174, 2009. Briefly, the Web MicroRNA Designer (WMD3) website (Available at wmd3 DOT weigelworld DOT org under cgi-bin/webapp DOTcgi) was used to design two sets of oligos complementary to different locations in the “common region” of the three BST coding sequences. Two independent constructs were designed and cloned into the pChlamyRNA3int plasmid obtained from the Chlamydomonas resource center. The oligos designed to target the three bestrophins that were used for RNAi knockdown are shown in Table 3. Two triple knockdown lines (BST-RNAi lines 1 and 2 or bsti-1 and bsti-2) were isolated from 400 transformants. The oligos B1 Forwards and Reverse were used to generate bsti-1 and the oligos B2 Forwards and Reverse were used to generate bsti-2.

TABLE 3
Oligos designed to target the three bestrophins.

| Oligo name | Oligo sequence |
|---|---|
| B1 Forwards | CTAGTGGGAGCGAGTTGCAAGGCATATCTCGCT |
| | GATCGGCACCATGGGGGTGGTGGTGATCAGCGC |
| | TATATGTTTTGCAACTCGCTCCCG |
| | (SEQ ID NO: 126) |
| B1 Reverse | CTAGCGGGAGCGAGTTGCAAAACATATAGCGCT |
| | GATCACCACCACCCCCATGGTGCCGATCAGCGA |
| | GATATGCCTTGCAACTCGCTCCCA |
| | (SEQ ID NO: 127) |
| B2 Forwards | CTAGTGAGAGCGTGTTGCAAGGCATATCTCGCT |
| | GATCGGCACCATGGGGGTGGTGGTGATCAGCGC |
| | TATATGTTTTGCAACACGCTCTCG |
| | (SEQ ID NO: 128) |
| B2 Reverse | CTAGCGAGAGCGTGTTGCAAAACATATAGCGCT |
| | GATCACCACCACCCCCATGGTGCCGATCAGCGA |
| | GATATGCCTTGCAACACGCTCTCA |
| | (SEQ ID NO: 129) |

The pChlamyRNA3int plasmid carrying the AphVII gene that confers paromomycin resistance (para^{R}) was transformed into D66 by electroporation (Shimogawara et al., Genetics 148(4):1821-1828, 1998). Transformants were selected on TAP agar media containing the antibiotic paromomycin (4 μg mL⁻¹; Invitrogen). Resistant strains were then screened for “sick on low CO₂” phenotype by replica plating them on MIN plates. These were then placed in a high CO₂ chamber (5% [v/v]) CO₂ in air and a low CO₂ chamber (0.01% [v/v] CO₂ in air) with continuous illumination (100 μmol m⁻² s⁻¹) for 7 days. Spot tests were done by suspending growing cells in liquid MIN medium to same cell density (OD₇₃₀=0.1, 0.05 and 0.025) and 15 μL was spotted onto MIN plates. These plates were placed in high, ambient and low CO₂ chambers for 7 days. The CO₂ concentration was measured using an Environmental Gas Monitor (EGM-4, PP systems, Massachusetts).

Quantitative RT-PCR (qRT-PCR;qPCR): RNA extraction was carried out using the Trizol reagent (Invitrogen) according to manufacturer's instructions. 1 ug RNA per sample was used as template for cDNA, which was made using ProtoScript® First Strand cDNA Synthesis Kit (NEB) as per manufacturer's instructions. 100 ng RNA per sample was used to conduct qRTPCR using the Luna® Universal One-Step RT-qPCR Kit from NEB as per manufacturer's instructions using QuantStudio 6. The primers used for qPCR are listed in Table 4; CBLP primers were used as a control.

TABLE 4
Primers used for qPCR analysis.

| Gene target | Primer name | Primer sequence |
|---|---|---|
| BST1 | qBST1F | GCTGTGTGGCATTGAGGAGA |
| | | (SEQ ID NO: 130) |
| | qBST1R | GGATGAGGCTGATGAGTCCG |
| | | (SEQ ID NO: 131) |
| BST2 | qBST2F | ACGGTCTACGACTTCCCTCA |
| | | (SEQ ID NO: 132) |
| | qBST2R | TTGGATCACGTGGGATTGGG |
| | | (SEQ ID NO: 133) |
| BST3 | qBST3F | AAGTCAGCAAGGTTCCCTCG |
| | | (SEQ ID NO: 134) |
| | qBST3R | TGAATGAGCCTAGCGGGTTG |
| | | (SEQ ID NO: 135) |
| CBLP | qCBLPF | ATGTGCTGTCCGTGGCTTTC |
| | | (SEQ ID NO: 136) |
| | qCBLPR | CAGACCTTGACCATCTTGTCCC |
| | | (SEQ ID NO: 137) |

Growth phenotyping under different pH and CO₂ levels: The WT strain D66, the mutant strain cia3 (CAH3 knockout), the mutant strain pmp1 (LCIB/LCIC knockout), and bsti-1 were grown under very low CO₂ (0.01% CO₂ (v/v) in air), low (or ambient) CO₂ (0.04%-0.045% CO₂ (v/v) in air), and high CO₂ (5% CO₂ (v/v) in air). Growth in each of these three CO₂ conditions was tested at both pH 7 and pH 8.4. In order to inoculate the plates, three different cell concentrations were used; the highest concentration was 10,000 cells, the medium concentration was 5,000 cells, and the lowest concentration was 2,500 cells. Three spots at the three different concentrations were applied to each test plate for all four strains. The growth phenotyping experiment was replicated three times.

### Results

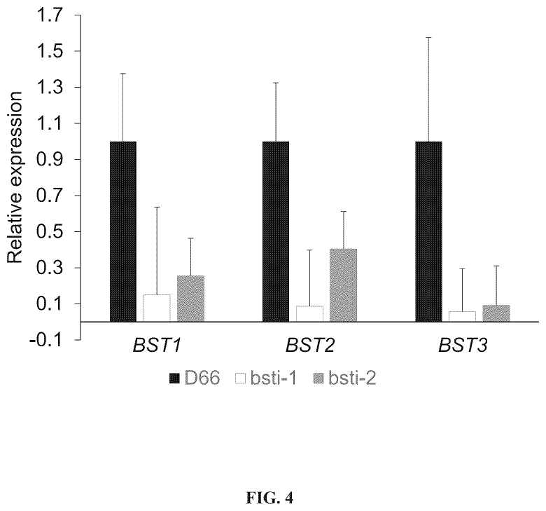

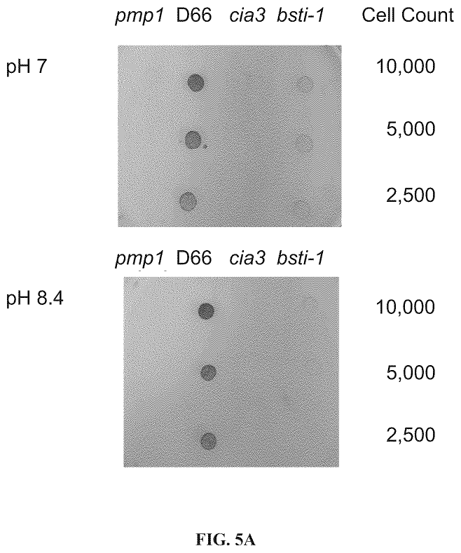

To examine the role of bestrophins in C. reinhardtii growth under low CO₂ conditions, an RNAi approach was adopted to reduce the expression of all three bestrophin genes at once. RNAi targeting BST1, BST2, and BST3 was used to generate two RNAi knockdown lines, bsti-1 and bsti-2. The expression levels of BST1, BST2, and BST3 in bsti-1, bsti-2, and in a WT control strain D66 were measured using quantitative RT-PCR. As shown in FIG. 4, the expression levels of all three bestrophin genes were lower in the bsti-1 and bsti-2 RNAi knockdown lines as compared to the WT control strain D66 (the error bars shown indicate the standard error for three biological replicates). In particular, bsti-1 and bsti-2 (BST-RNAi lines 1 and 2) showed nearly a 60-90% knockdown in the expression of BST1, BST2, and BST3 compared to D66. This result indicates that expression of all three bestrophin genes in the bsti-1 and bsti-2 RNAi knockdown lines were effectively reduced using the RNAi approach.

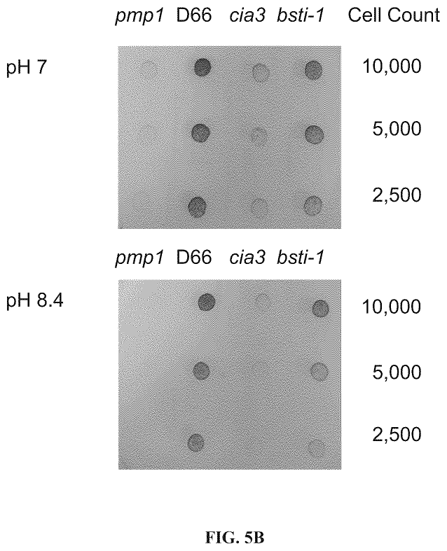

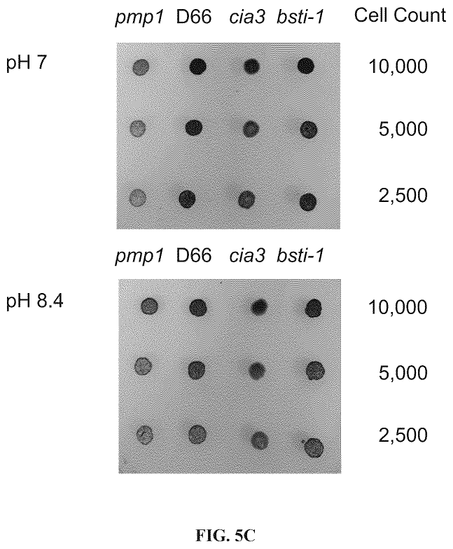

Next, the bsti-1 RNAi knockdown line, the wild type strain D66, along with the CAH3 mutant strain cia3, and the LCIB/LCIC mutant strain pmp1 were tested for their abilities to grow under different pH and CO₂ levels. The cultures were grown at a pH of 7 or 8.4, and under very low CO₂, low CO₂, or high CO₂ conditions. In very low CO₂ bsti-1 showed severely reduced growth that was further exacerbated at high pH, resembling the growth of CCM mutants, cia3 and pmp1 (FIG. 5A). At pH 7, the growth of bsti-1 was slightly better than at pH 8.4. In low CO₂, bsti-1, cia3, and pmp1 all showed reduced growth as compared to the wild type D66 strain (FIG. 5B). Again, all four strains showed slightly better growth under pH 7 as compared to pH 8.4. At high CO₂, however, the growth of bsti-1 was comparable to wild-type, cia3 and pmp1 (FIG. 5C). Taken together, these results show that the RNAi knockdown of all three bestrophins (bsti-1) shows a clear sick on low CO₂ phenotype as compared to both WT (D66) and other mutants with known sick on low CO₂ phenotypes (cia3, pmp1). Moreover, CAH3 (cia3 is the CAH3 knockout) is the carbonic anhydrase found within the C. reinhardtii thylakoid lumen, and is required for the functioning of the CCM. The three bestrophin proteins may be the proteins that deliver bicarbonate to CAH3 inside the thylakoids. Thus, these results show that all three BSTs are required for wild-type like growth of C. reinhardtii under low CO₂ conditions.

### Example 6: Triple Bestrophin RNAi Knockdown Strains have a Reduced Capacity to Accumulate Inorganic Carbon (Cᵢ)

The following example describes inorganic carbon affinity testing of the triple bestrophin RNAi knockdown strains bsti-1 and bsti-2 (described in Example 5) as compared to the WT strain D66.

### Materials and Methods

Affinity for inorganic carbon: For this assay, the rate of photosynthesis (as O₂ evolution) was measured at various inorganic carbon levels (inorganic carbon=CO₂ & HCO₃⁻). The affinity for external Cᵢ (K_{1/2} [DIC]) (dissolved inorganic carbon) was estimated according to Ma et al., Plant Physiol 156:884-896, 2011. Specifically, cells with an equivalent of 100 μg of chlorophyll were suspended in HEPES-NaOH buffer (pH 7.4), HEPES-NaOH buffer (pH 7.8), or 25 mM EPPS-NaOH buffer (pH 8.4) bubbled with inert nitrogen gas, i.e. CO₂ free. The cells were transferred to an O₂ electrode chamber (Rank Brothers, Cambridge UK) illuminated at 300 μmol m⁻² s⁻¹, and left to deplete any remaining DIC in the buffer and intracellular spaces. Upon depletion of endogenous CO₂, no net O₂ evolution is observed. Known concentrations of NaHCO₃ were injected into the chamber and the rate of O₂ evolution was measured. In this experiment, the inorganic carbon level was varied from 25 μM to 2 mM. The K_{1/2} [DIC] was calculated as the DIC concentration required for half (50%) maximal rates of oxygen evolution (i.e., photosynthesis) (Badger, 1985). Chlorophyll content was measured by combining chlorophyll a and b. Chlorophyll was extracted in 100% methanol and measured using the spectrophotometer. The K_{1/2} (CO₂) is taken as the CO₂ concentration needed to reach half Vₘₐₓ O₂ evolution.

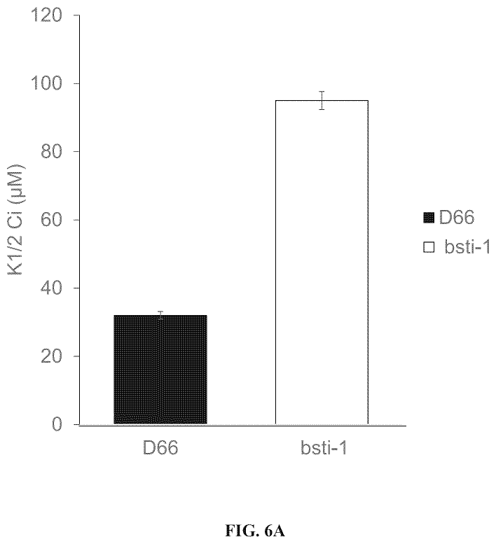

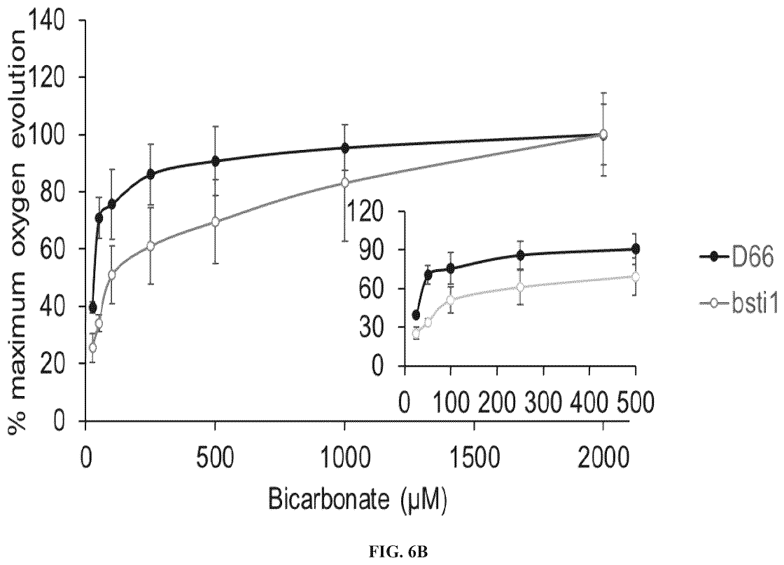

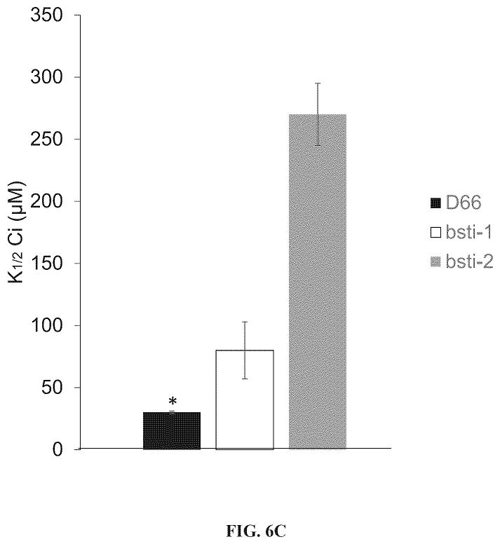

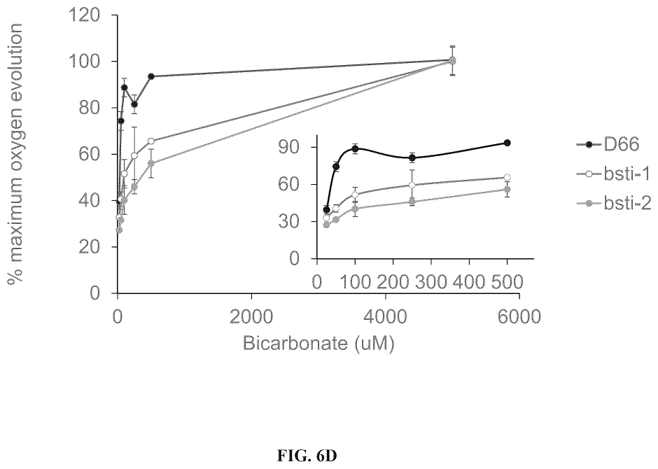

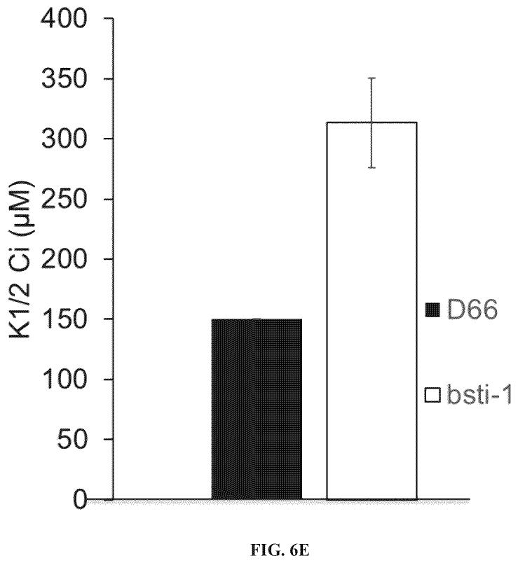

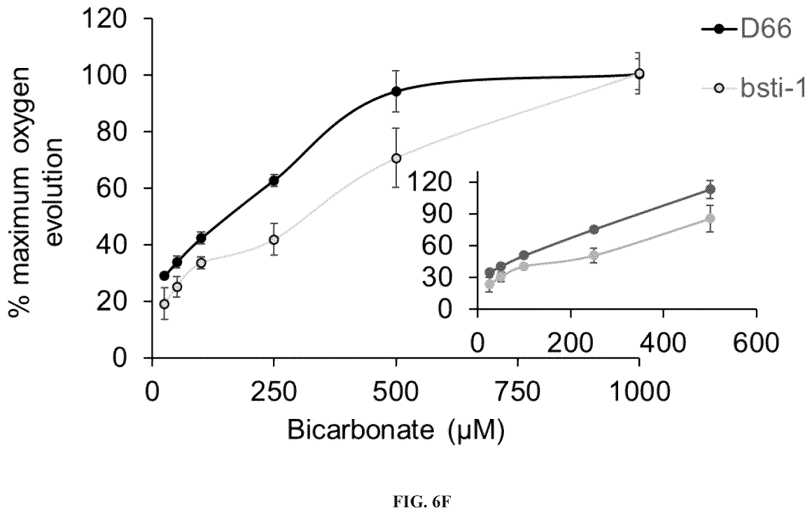

Cᵢ affinity was estimated for bsti-1 and D66 acclimated to low CO₂ (<0.04% CO₂) for 12 hours at pH 8.4 (FIG. 6A). The K_{0.5} (Cᵢ) values (Cᵢ concentration needed for half maximum oxygen evolution) shown in FIG. 6A was calculated from the O₂ evolution versus Cᵢ curves shown in FIG. 6B. In addition, Cᵢ affinity was estimated for bsti-1, bsti-2, and D66 acclimated to low CO₂ (<0.04% CO₂) for 12 hours at pH 7.8 (FIG. 6C). The K_{0.5} (Cᵢ) values (Cᵢ concentration needed for half maximum oxygen evolution) shown in FIG. 6C was calculated from the O₂ evolution versus Cᵢ curves shown in FIG. 6D. Further, Cᵢ affinity was estimated for bsti-1 and D66 acclimated to high CO₂ (>5% CO₂) for 12 hours at pH 7.8 (FIG. 6E). The K_{0.5} (Cᵢ) values (Cᵢ concentration needed for half maximum oxygen evolution) shown in FIG. 6E was calculated from the O₂ evolution versus Cᵢ curves shown in FIG. 6F. Triplicate runs were made at each Cᵢ concentration. The symbol “*” indicates that the differences in K_{0.5}(Cᵢ) was significant (P<0.05 by student t test). At pH 7.8 and low CO₂ acclimation, the Vmax of D66 is 121 μmol O₂ mg⁻¹ Chl hr⁻¹, the Vmax of bsti-1 is 105 μmol O₂ mg⁻¹ Chl hr⁻¹, and the Vmax of bsti-2 is 95 μmol O₂ mg⁻¹ Chl hr⁻¹. At pH 7.8 and high CO₂ acclimation, the Vmax of D66 is 121 μmol O₂ mg⁻¹ Chl hr⁻¹ and the Vmax of bsti-1 is 120 μmol O₂ mg⁻¹ Chl hr⁻¹. At pH 8.4 and low CO₂ acclimation, the Vmax of D66 is 124 μmol O₂ mg⁻¹ Chl hr⁻¹ and the Vmax of bsti-1 is 85.5 μmol O₂ mg⁻¹ Chl hr⁻¹. The Vmax of all the strains was set to 100% oxygen evolution activity.

Uptake of inorganic carbon: Silicone oil centrifugation was used to measure intracellular concentration of dissolved Cᵢ as per Moroney et al., Plant Physiol 79(1):177-183, 1985. Briefly, cells were centrifuged and suspended at 25 μg Chl mL⁻¹ density in Cᵢ depleted 25 mM EPPS-NaOH (pH 7.8 or 8.4) and incubated in the light until net O₂ evolution was zero. Cells were maintained in the light until used. 300 μL of Cᵢ depleted cells were then centrifuged in tubes containing 25 μL of 1 M glycine (pH 10) with 0.75% (w/v) SDS overlaid with 75 μL of Dow Corning AR 20 silicone oil. Assays were performed at 25° C. in 200 μmol m⁻² s⁻¹ light in a Beckman Microfuge B. Cᵢ uptake was initiated by adding either 3 μl of 25 mM (for an added concentration of 25 μM) at pH 7.8 or 50 mM ((for an added concentration of 50 μM) at pH 8.4 of NaH¹⁴CO₃ followed by the indicated time of illumination (between 15 and 120 sec at 150 μmol m⁻² s⁻¹ light). Triplicate samples were run for each time point. The reaction was terminated by a 15 second centrifugation in a microfuge B (Beckman). Internal Cᵢ was calculated using the difference between total and acid stable ¹⁴C in the pellet and corrected for cell volume as described (Machingura et al., J Exp Bot 68(14):3879-3890, 2017).

### Results

Two characteristics of algal cells with a CCM are first, their very high affinity for inorganic carbon (Cᵢ), and second, their ability to accumulate Cᵢ to levels higher than can be obtained by diffusion. Therefore, the photosynthetic oxygen evolution activity of the RNAi knockdown lines bsti-1 and bsti-2, and the WT strain D66 were tested. The RNAi knockdown lines bsti-1 and bsti-2 acclimated to low CO₂ exhibited a three to tenfold lower affinity for Cᵢ at pH 7.8, respectively, as judged by their measured K_{0.5}(Cᵢ) (FIG. 6A and FIG. 6C). When acclimated to high CO₂, bsti-1 also had a reduction in Cᵢ affinity compared to D66 (FIG. 6E). This indicates that reducing the expression of all three BST genes caused a reduction in the cells' affinity for Cᵢ. At pH 8.4, the K_{0.5}(Cᵢ) for bsti-1 is 95 M, in sharp contrast to a low K_{0.5}(Cᵢ) of 35 μM for D66. At the higher pH of 8.4, the predominant Cᵢ species in the medium would be bicarbonate, thus the higher affinity of the D66 (WT) cells for Cᵢ reflects their ability to actively take up and utilize bicarbonate. These results, which show that bsti-1 and bsti-2 have a higher requirement for inorganic carbon for photosynthetic O₂ evolution, are strong evidence for a role of the bestrophins in the CCM.

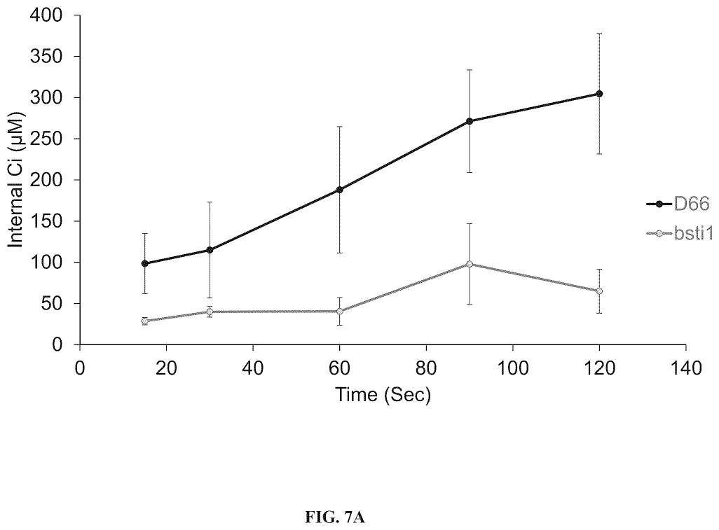

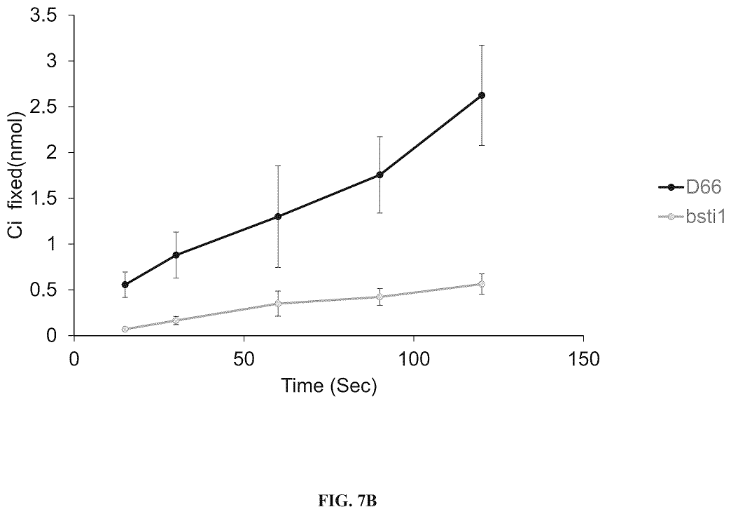

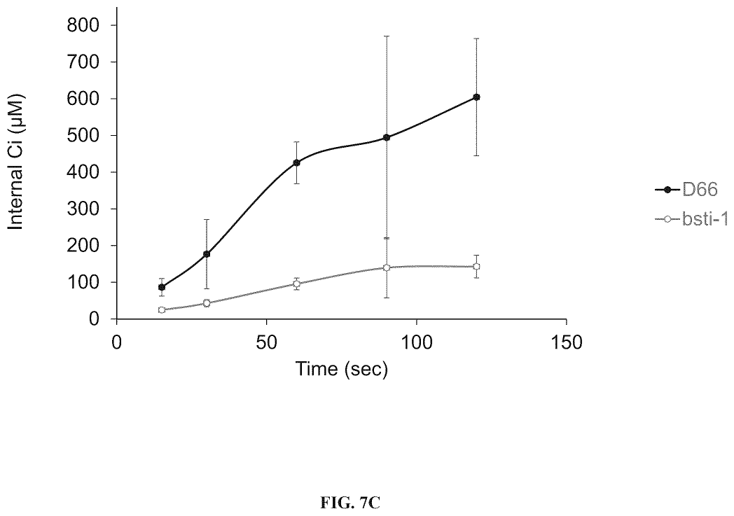

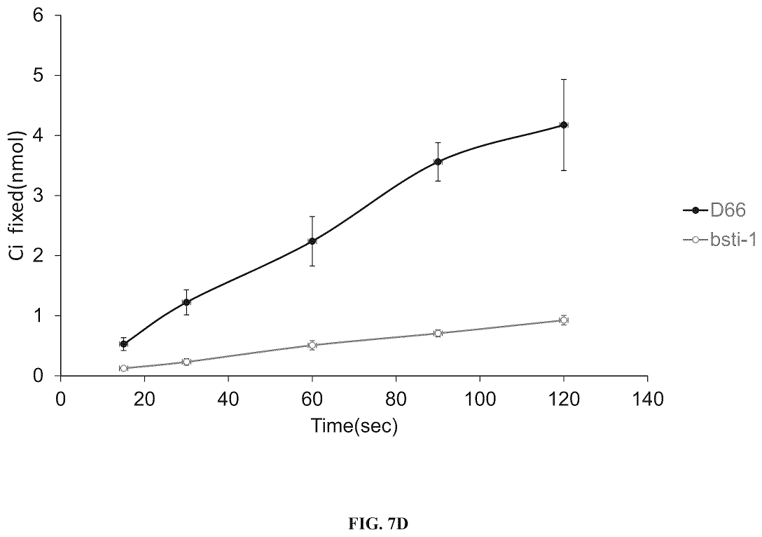

Cᵢ uptake activity was also measured in D66 and bsti-1 to evaluate the importance of BST1-3 in accumulation and fixation of Cᵢ. Low CO₂ acclimated bsti-1 had significantly lower accumulation and fixation of ¹⁴Cᵢ compared to D66 at both pH 7.8 as well as pH 8.4 (FIG. 7A-7D). At both pH 7.8 and 8.4, bsti-1 accumulated ¹⁴Cᵢ to only 20 to 25% of the levels observed in D66 cells. This difference was seen at both the earliest time point (15 seconds) and at the latest time point (up to two minutes in the light) where the D66 cells had used up most of the added ¹⁴Cᵢ. These results indicate that BST1-3 have an important role to play in the Cᵢ uptake in low CO₂ conditions in C. reinhardtii, and again provide strong evidence for a role of the bestrophins in the CCM.

### Example 7: The Bst3 Knockout Strain has Normal Growth Under Low CO₂ Conditions and Similar Inorganic Carbon Affinity when Compared to WT

The following example describes growth under low CO₂ conditions and Cᵢ affinity testing of the C. reinhardtii bst3 knockout strain as compared to the WT strain D66.

### Materials and Methods

C. reinhardtii knockout strains: Knockout strains are created using random insertional mutagenesis, and are part of the Chlamydomonas Library Project (CLiP). Strains can be ordered from the Chlamydomonas Resource Center (Available at www DOT chlamycollection DOT org). At present, only a bst3 knockout strain is available; bst1 and bst2 knockout strains are not available. In order to confirm the insert position in the bst3 knockout strain, PCR was performed using primers specific for the BST3 gene and for the insert (see Table 5). Insert specific primers (CIB1F and CIB1R) and the information for the flanking region were obtained from the CLiP website (Available at www DOT chlamylibrary DOT org under allMutants).

TABLE 5
Primers used to analyze the bst3 insertion.

| Primer name | Primer sequence |
|---|---|
| BST3F | TGCCCCTTCTCAGCACGT (SEQ ID NO: 138) |
| BST3R | ACTGCCTCACACTCCCCT (SEQ ID NO: 139) |
| CIB1F | GCACCAATCATGTCAAGCCT (SEQ ID NO: 140) |
| CIB1R | GACGTTACAGCACACCCTTG (SEQ ID NO: 141) |

Semi-quantitative RT-PCR analysis was performed as in Example 3.

Growth measurement: WT strain D66 and knockout strain bst3 were grown in pH 8.6, using methods described in the above Examples. Growth was measured using OD₇₃₀, and chlorophyll estimation was done at wavelengths 645 and 663. Measurements were done at low CO₂ (<0.04% CO₂) for six days. Cells were grown in TAP for 48 hours before transferring them to MIN at an OD₇₃₀ of 0.01.

Affinity for inorganic carbon: This was done using methods described in Example 6. Oxygen evolving activity was measured at pH 7.4 and the K_{0.5}(Cᵢ) values (Cᵢ concentration needed for half maximum oxygen evolution) were calculated from the O₂ evolution versus Cᵢ curves. Triplicate runs were made at each Cᵢ concentration.

### Results

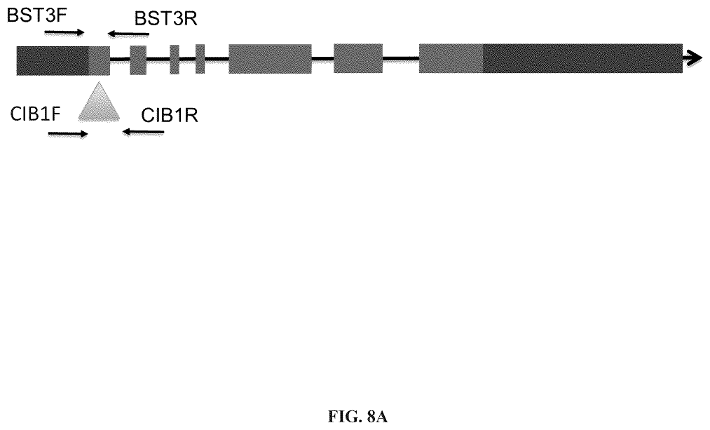

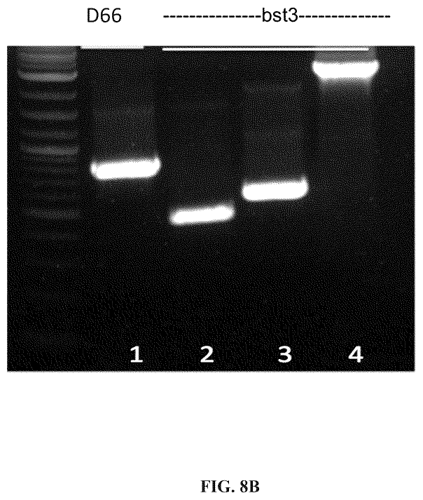

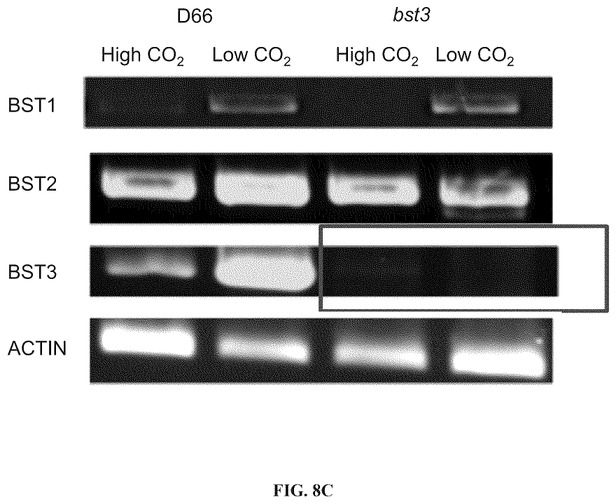

A BST3 knockout (bst3) was obtained from the CLiP mutant collection with a paromomycin insert in the first exon of the bst3 gene (FIG. 8A). The location of the insert was confirmed using PCR (FIG. 8B). In addition, BST1-3 expression was analyzed in the bst3 knockout strain under high and low CO₂ conditions, and compared to the WT strain D66. BST3 expression was not detected in bst3 under both conditions, while BST1 and BST2 expression were comparable between the bst3 knockout strain and the WT strain D66 under both conditions (FIG. 8C).

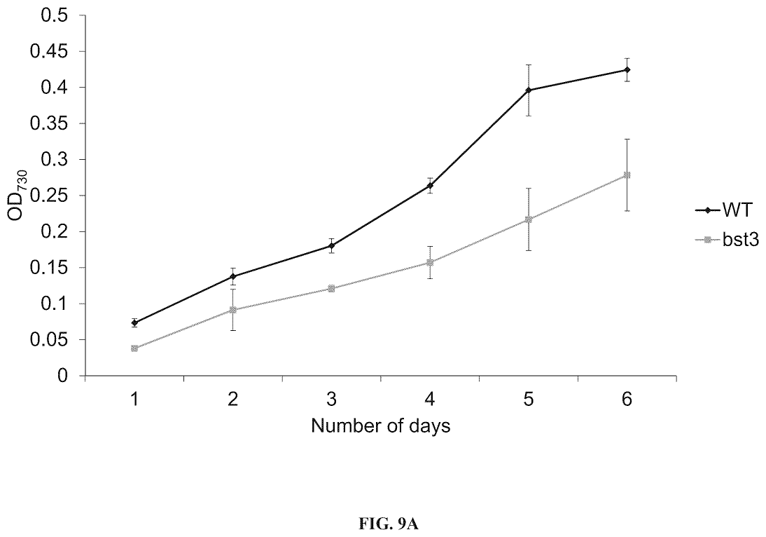

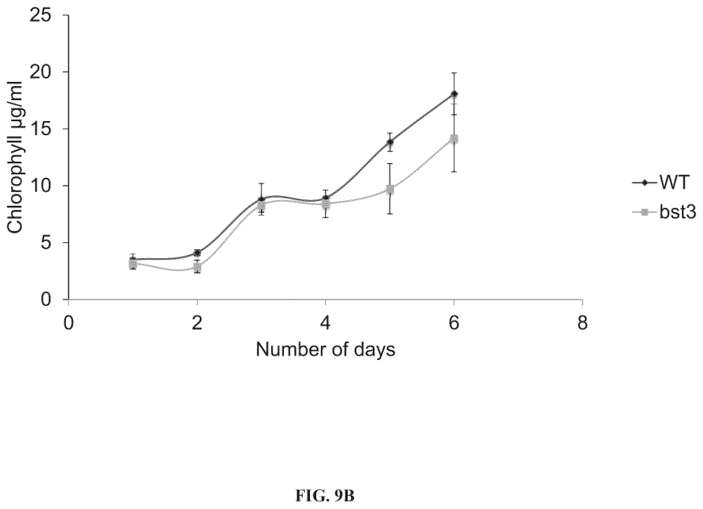

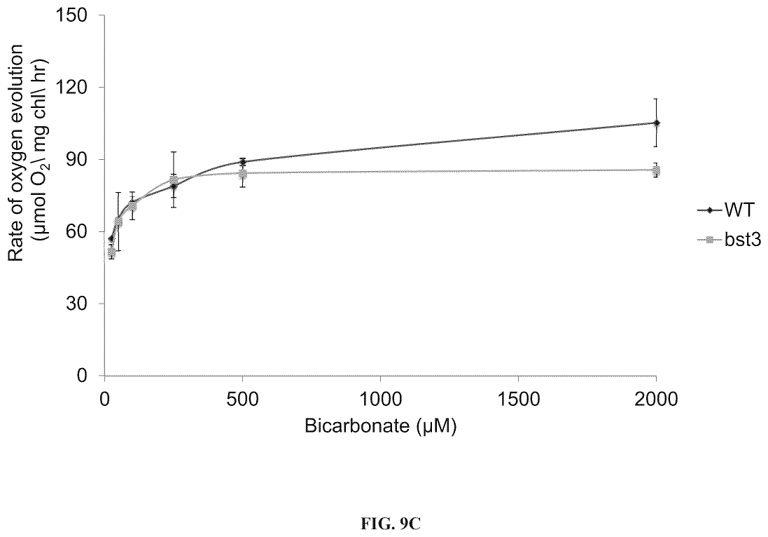

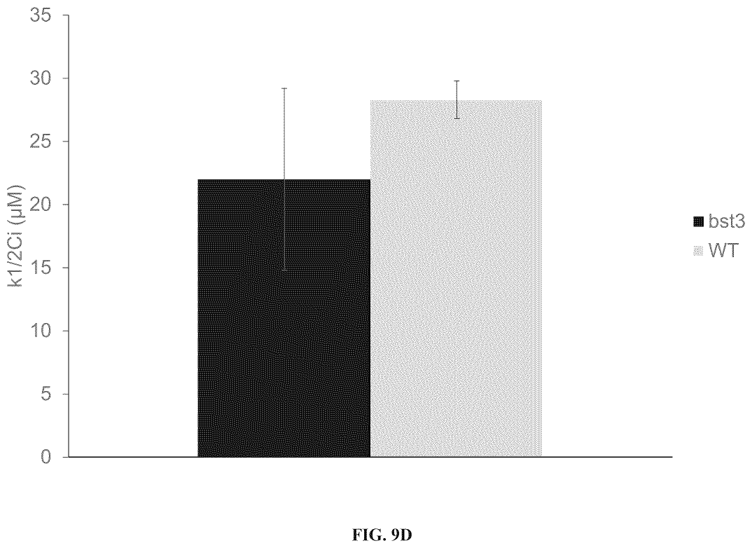

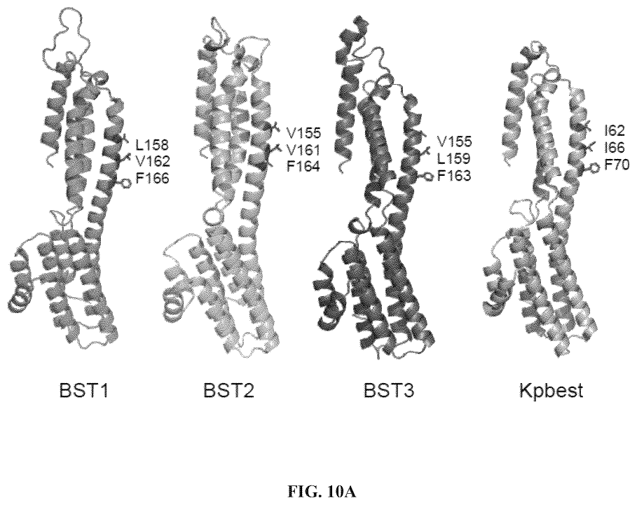

No significant growth difference for the bst3 strain was observed as compared to wild-type cells under low (<0.04% CO₂) (FIGS. 9A-9B), very low (0.02% CO₂) or high CO₂ (5% CO₂) (v/v) in air (data not shown). There also was no notable difference in Cᵢ affinity between WT and bst3 (FIG. 9C). In the RNAi knockdown line bsti-1, where the expression of all three BST genes is reduced, there is a reduced Cᵢ affinity at both pH 7.8 and pH 8.4. This is in stark contrast to bst3, the knockout strain only missing BST3 expression, which has no difference in Cᵢ affinity with WT (FIG. 9C-9D). These results support the hypothesis that the function of the three BSTs is redundant, and that the expression of all three genes must be reduced to observe physiological effects.

### Example 8: Structural Characterization of BST1, BST2, and BST3 and Current C. reinhardtii CCM Model

The following example describes the structural characterization of the C. reinhardtii BST1, BST2, and BST3 proteins. The example also describes the current C. reinhardtii CCM model.

### Materials and Methods

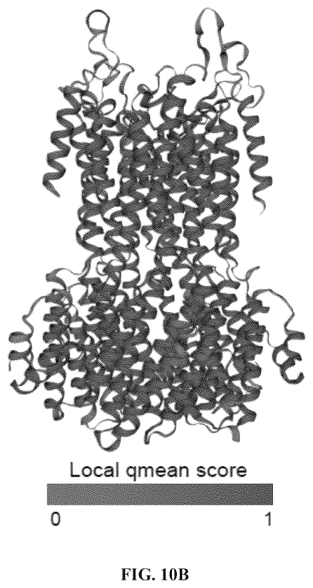

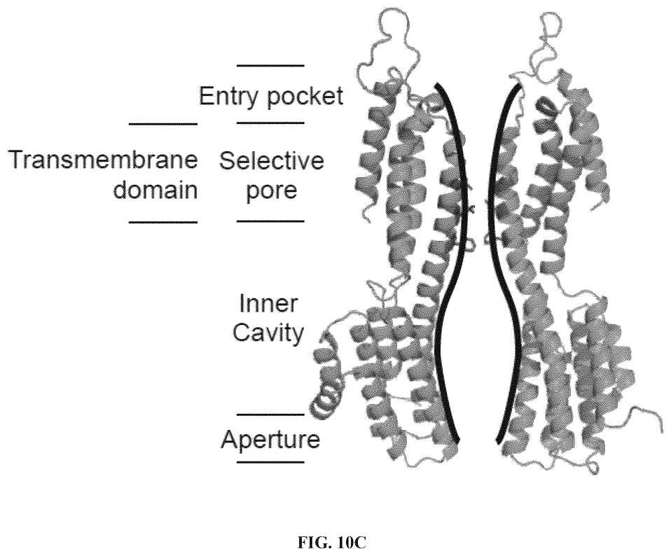

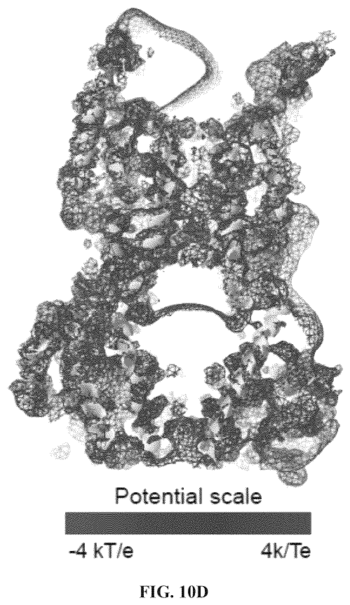

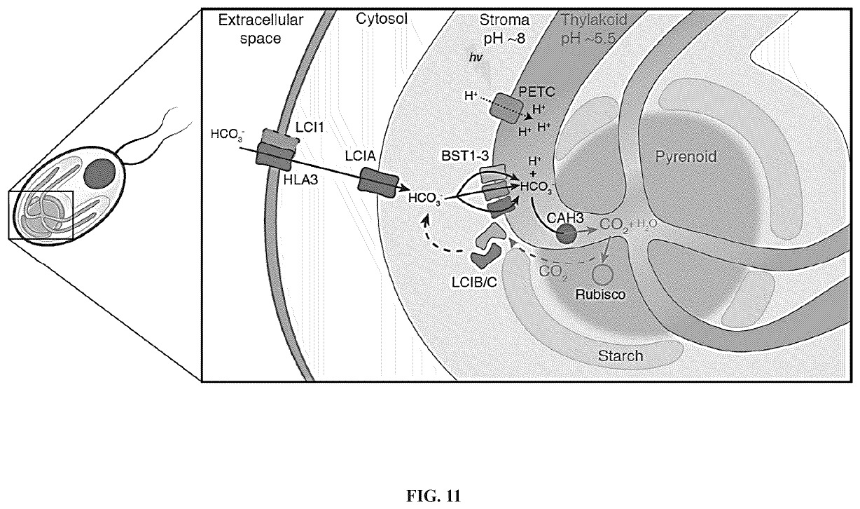

Peptide sequences of BST1, BST2, and BST3 were obtained from Phytozome v12.1. Homology modelling was done using Klebsiella pneumonia bestrophin (Kpbest; PDB: 4DW8) as a template, and generated with Swiss-Model webserver (Yang, et al., Science 346(6216):1498-1501, 2014). Kpbest was chosen because it was identified as the highest ranking template for BST1-3 by Swiss-Model. The obtained structures and that of Kpbest are displayed as monomers with conserved residues lining the selective pore highlighted in red and labeled in FIG. 10A. The obtained BST1 homopentamer model was submitted to energy minimization with gromos 43B1 forcefield and the electrostatic potential was calculated using atom partial charge using Swiss-PDBviewer (V4.01). Then, the electric field was displayed on the BST1 homopentamer model to generate FIG. 10D. The electrostatic potentials were displayed in potential scale of −4 kT/e (red, negative) to +4 kT/e (blue, positive), and the cavity was highlighted in green. The BST1 homopentamer structure quality (FIG. 10B) was obtained using the QMEAN score (Benkert et al., Proteins 71(1):261-277, 2008).

### Results

To predict whether BST1-3 might function as negative ion transporting bestrophins, homology modelling of BST1-3 using Klebsiella pneumonia bestrophin was done. BST1-3 contain non-polar residues along their selective pore that are conserved in proteins of the bestrophin family (FIG. 10A; Qu et al., J Neurosci 26(20):5411-5419, 2006). Structural studies show that human and K. pneumonia bestrophins are pentameric, and modelling of BST1 in a pentameric assembly is of high confidence (FIG. 10B). The entry pocket is has a predominantly negative electrostatic potential and the selective pore is neutral/positively charged supporting the hypothesis that BST1-3 transport negatively charged ions (FIGS. 10C-10D; Yang, et al., Science 346(6216):1498-1501, 2014; Kane et al., Nature 516(7530):213-218, 2014). These results suggest that C. reinhardtii bestrophins may be anion transporters.

The results described in Examples 2-8 demonstrate an important role for the three bestrophin proteins in the context of the C. reinhardtii CCM. Taken together, these results indicate that the three bestrophin proteins are thylakoid-localized bicarbonate-transporting channels. FIG. 11 shows the integration of BST1, BST2, and BST3 into the current C. reinhardtii CCM model for Cᵢ transport.

1: A method of producing a genetically altered plant, comprising:
(a) introducing to a plant one or more genetic alterations comprising expression of a green algal bestrophin polypeptide localized to a chloroplast envelope and/or to a thylakoid membrane of at least one chloroplast of the plant, wherein the green algal bestrophin polypeptide transports bicarbonate across a membrane from a plant cell cytoplasm into a stroma of at least a portion of the chloroplasts of the plant and/or transports bicarbonate across a membrane from a stroma into a lumen of at least a portion of the chloroplasts of the plant; and
(b) cultivating the plant under conditions wherein the transport of bicarbonate into the stroma or into the lumen increases carbon use efficiency, increases water use efficiency, increases nitrogen use efficiency, reduces photoinhibition, increases growth, and/or increases productivity as compared to a corresponding wild-type plant grown under the same conditions that lacks the one or more genetic alterations.

2: The method of claim 1, wherein the green algal bestrophin polypeptide comprises at least 75% sequence identity to a sequence selected from the group consisting of: SEQ ID NO: 1, SEQ ID NO: 2, and SEQ ID NO: 3.

3: The method of claim 1, comprising expression of the green algal bestrophin in a leaf mesophyll cell.

4: The method of claim 1, comprising expression of the green algal bestrophin in at least 70% of leaf mesophyll cells of the plant.

5: The method of claim 1, wherein the plant is selected from the group consisting of cowpea, soybean, cassava, rice, soy, barley, wheat, and other C3 crop plants, and wherein the plant is not selected from the group consisting of corn, sorghum, and other C4 crop plants.

6: A method of increasing bicarbonate transport across a membrane from a stroma into a lumen of at least a portion of chloroplasts of a genetically altered plant, comprising:
(a) providing a genetically altered plant comprising one or more genetic alterations that express a green algal bestrophin polypeptide in a chloroplast envelope and/or in a thylakoid membrane of at least one chloroplast of the genetically altered plant; and
(b) cultivating the genetically altered plant under ambient carbon dioxide conditions;
wherein the green algal bestrophin polypeptide transports bicarbonate across a membrane from a plant cell cytoplasm into a stroma of at least a portion of the chloroplasts of the plant and/or transports bicarbonate across a membrane from a stroma into a lumen of at least a portion of the chloroplasts of the plant, and wherein the increase in bicarbonate transport is as compared to a corresponding wild-type plant cultivated under ambient carbon dioxide conditions.

7: The method of claim 6, wherein the green algal bestrophin polypeptide comprises at least 70% sequence identity to a sequence selected from the group consisting of: SEQ ID NO: 1, SEQ ID NO: 2, and SEQ ID NO: 3.

8: The method of claim 6, comprising expression of the green algal bestrophin in a leaf mesophyll cell.

9: The method of claim 6, comprising expression of the green algal bestrophin in at least 70% of leaf mesophyll cells of the plant.

10: The method of claim 6, wherein the plant is selected from the group consisting of cowpea, soybean, cassava, rice, soy, barley, wheat, and other C3 crop plants, and wherein the plant is not selected from the group consisting of corn, sorghum, and other C4 crop plants.

11: A genetically altered plant or part thereof, comprising a genetic alteration comprising expression of a polypeptide with at least 75% sequence identity to a sequence selected from the group consisting of SEQ ID NO: 1, SEQ ID NO: 2, and SEQ ID NO: 3, wherein the polypeptide transports bicarbonate and is localized to a chloroplast envelope and/or to a thylakoid membrane of at least one chloroplast of the genetically altered plant.

12: The genetically altered plant or part thereof of claim 11, wherein the plant or part thereof comprises increased carbon use efficiency, increased water use efficiency, increased nitrogen use efficiency, reduced photoinhibition, increased growth, and/or increased productivity as compared to a corresponding wild-type plant grown under the same conditions that lacks the genetic alteration.

13: The genetically altered plant or part thereof of claim 11, wherein the green algal bestrophin is expressed in a leaf mesophyll cell.

14: The genetically altered plant or part thereof of claim 11, wherein the green algal bestrophin is expressed in at least 70% of leaf mesophyll cells of the plant.

15: The genetically altered plant or part thereof of claim 11, wherein the plant is selected from the group consisting of cowpea, soybean, cassava, rice, soy, barley, wheat, and other C3 crop plants, and wherein the plant is not selected from the group consisting of corn, sorghum, and other C4 crop plants.

16: A transfected plant or part thereof comprising a transfected nucleic acid sequence comprising a coding sequence of a polypeptide with at least 75% sequence identity to a sequence selected from the group consisting of: SEQ ID NO: 1, SEQ ID NO: 2, and SEQ ID NO: 3 in the plant or part thereof; wherein the polypeptide transports bicarbonate and is expressed in the plant or part thereof.

17: The transfected plant or part thereof of claim 16, wherein the polypeptide is localized to a chloroplast envelope or a chloroplast thylakoid membrane of at least one chloroplast of a plant cell, wherein the plant cell is a leaf mesophyll cell, and wherein the polypeptide is expressed in at least 70% of leaf mesophyll cells of the plant.

18: The transfected plant or part thereof of claim 16, wherein the transfected nucleic acid sequence is stably integrated into the nuclear genome of the plant.

19: The transfected plant or part thereof of claim 16, wherein the transfected nucleic acid sequence further comprises a second nucleic acid sequence encoding a signal peptide sequence or targeting sequence operably linked to the coding sequence of the polypeptide, wherein expression of the signal peptide sequence or targeting sequence results in localization of the polypeptide to a chloroplast envelope or a chloroplast thylakoid membrane of at least one chloroplast of a plant cell.

20: The transfected plant or part thereof of claim 16, wherein the polypeptide comprises SEQ ID NO: 2 or SEQ ID NO: 3.

21: The transfected plant or part thereof of claim 16, wherein the plant is selected from the group consisting of cowpea, soybean, cassava, rice, soy, barley, wheat, and other C3 crop plants, and wherein the plant is not selected from the group consisting of corn, sorghum, and other C4 crop plants.

22: The transfected plant or part thereof of claim 16, wherein the plant comprises increased carbon use efficiency, increased water use efficiency, increased nitrogen use efficiency, reduced photoinhibition, increased growth, and/or increased productivity.
